(19) United States
(12) Patent Application Publication
(10) Pub. No.: US 2006/0198882 A1
(43) Pub. Date: Sep. 7, 2006

(54) Stable liposomes or micelles comprising a sphinolipid and a peg-lipopolymer

(76) Inventors: Yechezkel Barenholz, Jerusalem (IL); Elena Khazanov, Ramat Beit Schrmeb (IL); Joris Schillemans, Wijchen (NL)

Correspondence Address: BROWDY AND NEIMARK, P.L.L.C.;624 NINTH STREET, NW, SUITE 300, WASHINGTON, DC, 20001-5303, US

(21) Appl. No.: 10/551,649

(22) PCT Filed: Mar. 31, 2004
(86) PCT No.: PCT/IL04/00294

§ 371 Date: Sep. 29, 2005

(30) Foreign Application Priority Data
Mar. 21, 2003 (US) 60458404

(51) Int. Cl. A61K 39/395; A61K 9/127

(52) U.S. Cl. 424/450; 424/178.1

(57) ABSTRACT

The present invention concerns a stable lipid assembly comprising a biologically active lipid having a hydrophobic region and a polar headgroup, wherein the atomic mass ratio between the headgroup and hydrophobic region is less than 0.3, and a lipopolymer having a hydrophobic lipid region and a polymer headgroup, wherein the atomic mass ratio between the headgroup and hydrophobic region is at least 1.5 and optionally a lipid matrix composed of liposome forming lipids. Specific lipid assemblies according to the invention comprise the biologically active lipid, ceramide, a lipid derivatized with polyethylene glycol (lipopolymer) and optionally in combination with a phospholipid (e.g. Egg phosphatidylcholine (EPC) and hydrogenated soybean phosphatidylcholine (HSPC)). The lipid assemblies of the invention exhibited a therapeutic effect in vitro in tumor cells as well as in vivo in animal models and they deliver the biologically active lipid to the disease site.

### FIELD OF THE INVENTION

This invention relates to lipid assemblies and in particular to lipid assemblies comprising a biologically active lipid which tends to aggregate in a polar environment, to a state other than liposomes.

### LIST OF PRIOR ART

The following is a list of prior art which is considered to be pertinent for describing the state of the art in the field of the invention.
Israelachvili, J. N. Physical principles of membrane organization, Q. Rev Biophys, 13(2):121-200 (1980)
Lichtenberg and Barenholz, In Methods of Miocheical Analysis, D. Glick (Ed), 33:337-462, (1988)
Kumar, Biophys J., 88:444-448, (1991)
Israelachvili, J. N., In Intermolecular and surface forces, 2^{nd} Ed. Academic Press, pp 341-365, (1992)
Barenholz and Cevc, In Physical Chemistry of Biological Surfaces, Marcel Dekker, NY, pp 171-241, (2000)
Seddon, J. M., Biochemistry, 29(34):7997-8002, (1990)
Lofgren and Pasher, Chem. Phys. Lipids, 20(4):273-284, (1977)
Carrer and Maggio, Biochim. Biophys Acta, 1514(1):87-99, (2001)
Mimeault, FEBS Letters, 530:9-16, (2002)
Saldeen et al., Cytokine, 12940:405-408, (2000)
Vento, R. M. et al. Mol. Cell. Biochem. 185:7-153 (1998)
Ogretmen, B. D. et al. J. Biol. Chem, 276:24901-24910 (2001)
Wyllie, A. H. et al. In: International Review of Cytology, G. H. Bourne, F. J.
Danielli, K. W. Jeon (Eds) New York: Academic Press 68:251-306 (1998)
Hannun Y. A. et al. Biochimica et Biophysica Acta 1585:114-125 (2002)
Cabot, M. C. and Giuliano A. E. Breast Cancer Res. Treat 46:46-71 (1997)
Ogretmen, B. D. et al J. Biol. Chem 276:24901-24910 (2001)
Mueller, H. and Eppenberger, U. Anticancer Res. 16:3845-3848 (1996)
Senchenkov, A. et al. J. Natl. Cancer Inst. 93:347-357 (2001)]
Cai Z., et al. J. Biol. Chem. 272:6918-6926 (1997)
Tirosh et al., Biopys. J., 74(3):1371-1379, (1998)
Shabbits J A and Mayer M D, BBA 1612(1):98-106 (2003)
Charles R, et al. Circ Res, 87: 282-288
Lavie Y, et al. J Biol. Chem, 271:19530-19536 (1996)
K. Endo et al., Cancer Res. 51:1613-1618, (1991)
Kumar W V, Proc Natl Acad Sci USA 88(2):444-448 (1991)
Zhang et al., J. Biol. Chem., 265:76-81, (1990)
Zhang et al., J. Cell Biol., 114:155-167, (1991)
Cuvillier et al., Nature, 381(6585):800-3, (1996)
Barenholz, Y. and Amsalem, S. In: Liposome Technology 2^{nd} Edn., G. Gregoriadis (Ed.) CRC Press, Boca Raton, 1993, vol. 1, pp: 527-616.
Zuidam, N. J. and Barenholz Y. Biochim. Biophys. Acta. 1329(2):211-222 (1997)
Garbusenko O, Barenholz Y and Priev A, submitted
Samuni A. M, et al. Free Radic Biol Med., 23(7):972-9 (1997)
Biltonen R. L. and Lichtenberg D. Chemistry and physics of lipids. 64(1-3):129-142 (1993)
Eggers F. and Funk. Rev. Sci. Instrum. 44:969-977 (1973)
Kell, G. S. J. Chem. Eng. Data. 20:7-108 (1975)
Barenholz Y. et. al. From Liposomes: a practical approach, 2^{nd} Edn., RRC New ed, IRL Press Oxford, 1997
Gorodetsky, R. et al. Int. J. Cancer. 75:635-642 (1998)
Bligh E. G. and Dyer, Can W. J. J. Biochem. Physiol. 37:9111-9117 (1959)
Reid, S et al. J. Immunol. Methods 192(1-2):43-54 (1996)
Jouvet, P. Mol. Biol. Cell 11:1919-1932 (2000)
Gavrieli Y. et. al. J Cell Biol. 119: 493(1992)
Hirabayashi et al., FEBS Letters, 358:211-214, (1995)
Hannun et al., Methods in Enzymology, 88:444-448, (2000)
Yechiel E. and Barenholz Y. J Biol Chem. August 5;260 (16):9123-31 (1985)
Thornberry, N. A. and Lazebnic, Y. Science 281:1312-1316 (1998)
Villa, P. et al. Trends Biochem. Sci., 22:388-393 (1997)
Ciesielska, E. et al. Cell. Mol. Biol. Lett., 5:441-450 (2000),
Liu, W. et al. Int. J. Cancer, 92:26-30 (2001)
Bellone, G. et al. Cancer Res., 21:2200-2206 (2001)
Gabizon A. et al., Cancer Res., 54:987-992, (1994)
Gabizon A, Shmeeda H, Barenholz Y. Clin Pharmacokinet. 42(5):419-36 (2003)
Amselem S., Cohen R.,. Barenholz Y., Chem. Phys. Lipids. 64:219-237 (1993)

### BACKGROUND OF THE INVENTION

Many lipids are directly or indirectly involved in signal transduction pathways that mediate cell growth, differentiation, cell death and many other cell functions, as exemplified by diacylglycerols (DAG), ceramides (Cer), sphingosine (Sph), sphingosine-1-phosphate (S1P), ceramide-1-phosphate (C-1-P), di- and trimethylsphingosine (DMS and TMS, respectively). Most of these lipids or their derivatives have the potential to have a therapeutic effect either as stand-alone drugs or as a support (by synergism) to other drugs. However, the main obstacle to such application in vivo is the ability to administer and/or to deliver these molecules in a way that will make them bioactive. Most of these bioactive lipids are not soluble in aqueous phase; some such as DAG and ceramides are difficult to disperse in a stable form in relevant media; some when dispersed as micelles (S1P, Sph) fall apart in biological fluids such as blood; most of them when incorporated into liposomes cause the liposome to be physically unstable.

Liposomes are sealed sacs in the micron and sub micron range dispersed in an aqueous environment in which one or more bilayers (lamellae) separate the external aqueous phase from the internal aqueous phase. The bilayer is composed of amphiphiles, the latter having a defined polar and apolar regions. When amphiphiles are present in an aqueous phase, they self aggregate such that their hydrophilic moiety faces the aqueous phase, while their hydrophobic domain is “protected” from the aqueous phase.

Liposomes have a number of properties that make them versatile drug carriers for either lipid-soluble or water-soluble drugs. Liposomal drug delivery systems markedly alter the bio-distribution of their associated drugs in a way controlled by liposome lipid composition and size. For example, using sterically stabilized liposomes (SSL) may delay drastically drug clearance, retard drug metabolism, decrease the volume of distribution, enable to control drug release when the liposomes are ≦150 nm in size, and may shift the distribution selectively in favor of diseased tissues having increased capillary permeability, such as cancer and inflammation sites.

Various approaches were proposed to classify amphiphiles into sub-groups. One approach is based on geometric and energetic parameters of amphiphiles. According to this approach proposed by Israelachvili and co-workers [[Israelachvili, J, Physical principles of membrane organization, Q. Rev Biophys, 13(2):121-200 (1980), Lichtenberg and Barenholz, In Methods of Miocheical Analysis, D. Glick (Ed), 33:337-462, 1988; Kumar, Biophys J., 88:444-448, (1991)] amphiphiles are defined by a packing parameter (PP), which is the ratio between the cross sectional areas of the hydrophobic and hydrophilic regions.
Amphiphiles with a packing parameter of ˜1.0 (cylinder-like molecules) form a lamellar phase and have a potential to form liposomes;
Amphiphiles with a larger packing parameter (inverted cone-shaped molecules) tend to form hexagonal type II (inverted hexagonal) phases.

Such amphiphiles when having very small headgroup disperse hardly and in some cases do not even swell in the aqueous phase;
Amphiphiles with a smaller packing parameter of ≧⅔ (cone-shaped molecules) will self-aggregate as micelles. Examples of micelle forming amphiphiles which self-aggregate include phospholipids with short hydrocarbon chains, or lipids with long hydrocarbon chains (<10 carbon atoms), but with large, bulky polar head-groups (e.g. gangliosides and lipopolymers composed of a lipid to which a polyethylene glycol (PEG) moiety (≧750 Da) is covalently attached) [Israelachvili, J. N., In Intermolecular and surface forces, 2^{nd} Ed. Academic Press, pp 341-365, (1992); Lichtenberg and Barenholz, Supra, (1988); Barenholz and Cevc, In Physical Chemistry of Biological Surfaces, Marcel Dekker, NY, pp 171-241, (2000)].

As a prerequisite in order to form liposomes, amphiphiles must be organized in a lamellar phase. However, the formation of lamellar phases is not sufficient to lead to liposome formation [Seddon, J. M., Biochemistry, 29(34):7997-8002, (1990)]. Liposome formation also requires the ability of the lamellae to close up on themselves to form vesicles. For example, some sphingolipids that form a lamellar phase are not able to form vesicles [Lichtenberg and Barenholz, Supra, (1988); Seddon, Supra, (1990); Barenholz and Cevc, Supra, (2000)].

One group of sphingolipids, which is of interest and cannot self-assembly to form stable liposomes include the Ceramides [Lofgren and Pasher, Chem. Phys. Lipids, 20(4):273-284, (1977); Carrer and Maggio, Biochim. Biophys Acta, 1514(1):87-99, (2001)]. Ceramides are lipids composed of fatty acids linked by an amide bond to the amino group of a long chain sphingoid base and are known to be key intermediates in the biosynthesis of sphingolipids.

In addition, the ceramide has been recognized as an important second messenger implicated in triggering apoptotic/necrotic processes in many cancer cell types. It was proposed that mechanism of cell death depends mainly on the specific stimulatory conditions and on the cell type [Mimeault, FEBS Letters, 530:9-16, (2002)]. For example, it was shown that the natural ceramide mainly induced necrotic cell death of RINm5F insulin-producing cells [Saldeen et al., Cytokine, 12940:405-408, (2000)].

The discovery of pro-apoptotic properties of ceramides [Vento, R. M. et al. Mol. Cell. Biochem. 185:7-153 (1998)] and recent finding that ceramide inactivates telomerase activity and, therefore, might be cancer-specific [Ogretmen, B. D. et al. J. Biol. Chem, 276:24901-24910 (2001)] made them an attractive candidates for antitumor therapy alone, as well as in combination with chemotherapeutic agents, in an attempt to overcome some of obstacles of chemotherapy.

As known, apoptosis (programmed cell death) is an active process, which is critical to the health of many organisms, in both embryogenesis and adult tissue homeostasis. Malfunction of apoptosis plays an important role in several disorders; in cancer and autoimmune diseases apoptosis is inhibited, while in neurodegenerative disease apoptosis occurs in an uncontrolled fashion. In both situations, control of apoptosis may reduce the disease symptoms. Because apoptotic cells are phagocytized and processed by macrophages, while necrotic cells release their constituents to the extracellular matrix producing inflammations and other local damage [Wyllie, A. H. et al. In: International Review of Cytology, G. H. Bourne, F. J. Danielli, K. W. Jeon (Eds) New York: Academic Press 68:251-306 (1998)] the use of ceramides as anti-tumor agents that selectively kill tumor cells by mean of apoptosis, avoiding the side effects of necrosis was further investigated.

The role of ceramide in apoptosis is discussed in numerous publications. Hannun Y. A et al. summarizes insights from studies of Cer metabolism, topology and effector action, identification of several genes for enzymes of ceramide metabolism, ceramide analysis etc. [Hannun Y. A. et al. Biochimica et Biophysica Acta 1585:114-125 (2002)].

Also, it was demonstrated that some chemotherapeutic drugs are cytotoxic due to elevation of intracellular level of ceramides. It was found that the widely used chemotherapeutic agent doxorubicin appears to be effective because of its ability to activate ceramide-mediated pathway. Exposure to doxorubicin increases ceramide levels in drug-sensitive tumor cells, but not in the doxorubicin-resistant tumor cells [Cabot, M. C. and Giuliano A. E. Breast Cancer Res. Treat 46:46-71 (1997)].

The demonstration of a role of ceramide in anti-proliferative processes [Ogretmen, B. D. et al. J. Biol. Chem 276:24901-24910 (2001)] implies that a defect in ceramide generation or in ceramide effector mechanisms could be involved in conferring a survival advantage to cancer cells. Other studies [Mueller, H. and Eppenberger, U. Anticancer Res. 16:3845-3848 (1996)] suggest that dysfunctional metabolism of ceramide which contributes to reduction in the level of ceramide is implicated in multi-drug (MD) resistance. A number of clinically important cytotoxic agents appear to be effective because of their ability to activate ceramide-activated pathways in cancer cells by activating ceramide synthase or sphingomyelinase enzymes, or by inhibition of glucosyl-ceramide synthase (GCS) activity. It was shown that TNF-α-resistant MCF-7 breast cancer cells have been characterized by inability of their sphingomyelinases to generate ceramide [Senchenkov, A. et al. J. Natl. Cancer Inst. 93:347-357 (2001)]. Also, the human ovarian adenocarcinoma cell line NIH:OVCAR-3, established from a patient resistant to doxorubicin, mephalan, and cisplatin, expresses high levels of glucosylceramide, which agrees with high levels of GCS [Z. Cai, Z. et al. J. Biol. Chem. 272:6918-6926 (1997)]. Thus, it was concluded that elevating intracellular ceramide levels, either by exogenous administration alone or in combination with chemotherapeutic agents, or by targeting ceramide metabolism and cell death signaling pathways, is an attractive clinical treatment strategy for therapy of sensitive tumors as well as for overcoming drug resistance.

However, an obstacle for therapeutic applications of ceramides resides in their physicochemical properties. For example, ceramides are highly hydrophobic and therefore indispersible in aqueous media, while DMS and sphingosine-1-phosphate have detergent properties and may damage biological membranes.

### SUMMARY OF INVENTION

The present invention concerns lipid assembly structures which includes a non-liposome forming lipid. This structure enables the in vivo delivery, via the novel assembly of biochemically and/or pharmaceutically and/or therapeutically significant lipids.

Thus, according to a first of its aspects, the present invention provides a stable lipid assembly comprising:
(a) a biologically active lipid having a hydrophobic region and a polar headgroup, wherein the atomic mass ratio between the lipid headgroup and lipid hydrophobic region is less than 0.3;
(b) a lipopolymer having a hydrophobic lipid region and a polymer headgroup wherein the atomic mass ratio between the polymer headgroup and hydrophobic region is at least 1.5.

Lipid assembly as used herein denotes an organized collection of lipids forming inter alia, micelles and liposomes.

Stable lipid assembly as used herein denotes an assembly being chemically and physically stable under storage conditions (4° C., in biological fluids) for at least six months. This term also encompass assemblies which in the presence of a lipopolymer, the biologically active, non-liposome forming lipid, has a low desorption rate from the lipid assembly and that during storage the integrity and composition of the lipid assembly is substantially unaltered. The stability of the assembly is accomplished by the combination of biologically active lipid as defined above with the lipopolymer, i.e. in the absence of the lipopolymer as defined above, a substantial portion of the biologically active lipid initially loaded into the assembly (i.e. upon formation of the assembly) is removed therefrom within a short time after storage and/or aggregation of lipids occurs. As a result, the assembly is either highly toxic or the injection dose does not carry sufficient (desired) amount of the biologically active lipid to the target site and the assembly is not effective to achieve the desired biological effect.

Biologically active lipid as used herein interchangeably with the term non-liposome forming lipid denotes naturally occurring, synthetic and semi-synthetic amphiphiles having a hydrophobic region, comprising one or more long acyl or alkyl chain groups and a polar, ionic or non-ionic headgroup, wherein the atomic mass ratio between the headgroup and hydrophobic region is less than 0.3. Such amphiphiles may also be defined by their geometrical structure, typically being in the shape of a truncated inverted cone. Alternatively, or in addition, non-liposome forming lipids may be defined by their packing parameter, being greater than 1. The biologically active lipid according to the invention tends to aggregate in a polar environment, to a state other than liposomes. These states include, for example, inverted micelles, inverted hexagonal phases or assemblies of a wide range of sizes or long and thin tubular structures or undefined precipitates. The biologically active lipids are typically embedded with their hydrocarbon chains in parallel to other components of the assembly.

The biological activity of the biologically active lipids according to the invention refers to any measurable regulatory or biochemical effect they exhibit on a biological target site to which it is delivered by the assembly of the invention. The biological target site according the invention may include a cell, tissue or organ or a component thereof (e.g. intracellular component). One example of a biological effect according to the invention includes the induction of apoptosis.

It should be noted that albeit the biological effect of the non-liposome forming amphiphiles employed by the present invention, the lipid assembly may be associates with additional therapeutically active molecules, e.g. with a low molecular weight drug, as discussed in further detail hereinafter.

“Lipopolymer” as used herein denotes a lipid substance modified at its polar headgroup with a hydrophilic polymer. The lipopolymer of the invention is further defined by the atomic mass ratio between the polymer headgroup and the lipid hydrophobic region, being at least 1.5. Preferably, the lipopolymers of the invention are such that the level of water tightly bound to the headgroup is about 60 molecules of water per lipopolymer molecule. The level of water tightly bound to the headgroup is determined as described in Tirosh O. et. al. [Tirosh O. et. al Biophysical Journal, 74, 1371-1379(1998)]. In general, Tirosh et al. show that the calculation of the accessible surface area of a lipopolymer, such as a PEG molecule, from the specific volume data for the PEG and its components is approximately three water molecules per PEG repeated unit. Thus, a whole ⁷⁵⁰PEG molecule, having a degree of polymerization of 15, binds ˜60 water molecules and ^{2k}PEG molecule, having a degree of polymerization of 46, binds ˜142 water molecules.

The polymer headgroup of the lipopolymer is typically water-soluble and may be covalently or non-covalently attached to a hydrophobic lipid region. The lipopolymers according to the invention are well known in the art and are tolerated in vivo without toxic effects (i.e. are biocompatible).

According to a second of its aspects, the present invention provides a pharmaceutical composition comprising an amount of a lipid assembly, the amount being sufficient to achieve a biological effect at a target site, the lipid assembly comprising:
(a) a biologically active lipid having a hydrophobic region and a polar headgroup, wherein the atomic mass ratio between the lipid headgroup and lipid hydrophobic region is less than 0.3;
(b) a lipopolymer having a hydrophobic lipid region and a polymer headgroup wherein the atomic mass ratio between the polymer headgroup and hydrophobic region is at least 1.5.

The pharmaceutical composition may include, in addition to the lipid assembly structure a therapeutically active agent (e.g. a drug). The therapeutically active agent may be free, or associated with the lipid assembly structure of the invention, or associated with a different delivery system (e.g. in a separate liposome).

The term “association with” as used herein denotes any type of interaction between the different components of the assembly, including between the biologically active lipid, the lipopolymer, the lipid matrix, etc. Accordingly, association with includes, without being limited thereto, encapsulation, adhesion, adsorption, entrapment (either within the inner or outer wall of a liposomal assembly or in an intraliposomal aqueous phase) or embedment in the lipid layer (e.g. embedded in the liposomal membrane).

According to a third of its aspects, the present invention provides a method for the treatment or prevention of a disease or disorder, the method comprises providing an individual in need of said treatment, in a manner so as to achieve a therapeutic effect, an effective amount of a lipid assembly or composition according to the invention, optionally in combination with one or more therapeutically active agent.

The one or more therapeutically active agent may be provided to the individual in need together with the lipid assembly of the invention, either in the same pharmaceutical composition or separate therefrom. Alternatively, it may be provided to the individual within a predefined interval.

The term “treatment or prevention” is used herein to denote the administering of a therapeutic amount of the lipid assembly comprising the biologically active lipid (and the other additional therapeutic agents, either associated with the lipid assembly or separate therefrom) which is effective to ameliorate undesired symptoms associated with a disease, disorder or pathological condition, to prevent manifestation of such symptoms before they occur, to slow down progression of a disease, disorder or pathological condition, to slow down deterioration of symptoms, to enhance the onset of a remission period of a disease, disorder or pathological condition, to slow down irreversible damage caused in a progressive chronic stage of a disease, disorder or pathological condition, to delay onset of a progressive stage, to lessen the severity or to cure a disease, disorder or pathological condition, to improve survival rate or more rapid recovery, or to prevent a disease, disorder or pathological condition, form occurring or a combination of two or more of the above.

The term “disease, disorder or pathological condition” as used herein denotes any condition that impairs the normal function of a cell, tissue or organ. Non-limiting examples include conditions resulting from dysregulation of ceramide production and/or metabolism. Dysregulation of ceramide production and/or metabolism has been implicated in a number of disease states including cancer, atherosclerosis, insulin resistance, diabetes and multi-drug resistance to chemotherapy [Shabbits J A and Mayer M D, BBA 1612(1):98-106 (2003; Charles R, et al. Circ Res, 87: 282-288 (2000); Lavie Y, et al. J Biol. Chem, 271:19530-19536 (1996)].

The therapeutic effect to be achieved by the lipid assembly of the invention may vary depending on the biochemical effect of the biologically active lipid. For example, ceramides, which are one example of a biologically active lipid according to the invention, are known to induce in some target cells programmed cell death. Thus, the therapeutic effect to be achieved by lipid assemblies comprising ceramides may include inhibition of cell proliferation of target cells.

The present invention also provides the use of the lipid assemblies as defined above for the preparation of a pharmaceutical composition.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIGS. 1A-1B are schematically illustrations of the geometrical shapes of some lipids and a lipid assembly according to the invention; FIG. 1A is a schematic illustration of different geometrical molecular shapes of lipids and their typical packing parameter defined by the ratio A/B: (I) a lipid having a cylindrical molecular shape having a packing parameter; (II) a lipid having an inverted cone shape; (III) a lipid having a shape of a cone; (IV) a schematic illustration of the alignment of a combination of lipids of I, II, and III; FIG. 1B is a different schematic illustration of a combination of a lipid matrix (group I above), such as HSPC or EPC; with a lipopolymers (group III above) such as ^{2k}PEG-DSPE and different ceramides such as C₂ Cer (C2), C₆ Cer (C6), or C₁₆ Cer (C16), (group II above).

FIGS. 2A-2D are bar graphs showing measurements of maximal incorporation (concentration in supernatant (sup) vs. concentration in pellet) of C₆ Cer into multi-lamellar vesicles (MLV) or large unilamellar vesicles (LUV) with the following EPC:C₆Cer:^{2k}PEG-DSPE formulations: 58.5:34:7.5 (FIG. 2A); 54.5:38:7.5 (FIG. 2B); 56:34:10 (FIG. 2C); and 52:38:10 (FIG. 2D).

FIGS. 3A-E describe the effect of ceramides at different mole fractions on the thermotropic behavior of HSPC, ^{2k}PEG-DSPE:ceramide dispersion (for more details see Materials and Methods).

FIG. 3A represents the effect of increasing mole % of C₂ Cer, C₆ Cer , C₁₆ Cer and of C_{18:1} Cer on the temperature in which maximal change in the heat capacity (defined as Tm); FIG. 3B represents the effect of increasing mole % of the same ceramide mole fraction of each of the 3 ceramides (C₂ Cer, C₆ Cer, C₁₆ Cer) on the temperature range (324° C. (on-set) −330° C. (off set)) of the gel to liquid crystalline (SO/LD) MLV phase transition, as determined by DSC. FIGS. 3C-3E describe the 1st derivative curves of absorbance as optical densisty (dOD/dT), where T is the temperature according to Kelvin scale (° K) of HSPC/^{2k}PEG-DSPE (95:5) lipid dispersions (MLV) with different ceramides at different mole % (0 mole %, 12.5 mole %, 25 mole %, 50 mole % or 75 mole %); lipid dispersions containing C₂ Cer (FIG. 3C), C₆ Cer (FIG. 3D) and C₁₆ Cer (FIG. 3E).

FIG. 4 presents differential scanning colorimetry (DSC) curves of HSPC/C₆ Cer (3:1) MLV containing various amounts of ^{2k}PEG-DSPE (0,5 and 10 mole %).

FIGS. 5A-5D present 1st derivative curves (dOD/dT) of HSPC:C₆ Cer containing various amounts of ^{2k}PEG-DSPE: MLV (FIG. 5A) or LUV (FIG. 5B) as well as the 1^{st} derivative curves of the optical density (dOD/dTm) of LUV comprised of HSPC/^{2k}PEG-DSPE (5 mole %) (FIG. 5C) and of HSPC/^{2k}PEG-DSPE (7.5 mole %) (FIG. 5D) with the indicated amounts of C₆ Cer.

FIGS. 6A-6B are bar graphs showing the influence of the type of the lipid matrix: EPC, (FIG. 6A); or HSPC, (FIG. 6B), alone or in combination with different ceramides (C₂ Cer (C2), C₆ Cer (C6) or C₁₆ Cer (C16)) with or without ^{2k}PEG-DSPE (PEG) on partial specific compressibility of LUV.

FIGS. 7A-7D are graphs representing IC₅₀ values of C₆ Cer (C6) alone or as part of a lipid assembly according to the invention (EPC:C⁶ Cer or HPC:^{2k}PEG-DSPE:C⁶ Cer) at the indicated ratios, on OV-1063 (FIGS. 7A and 7B) or C-26 (FIGS. 7C and 7D) tumor cell lines after 4, 24 and 72 hours of incubation, as measured by the MB assay.

FIG. 8A-8B presents the level of radiolabelled C₆ Cer (C6) or its metabolite products sphingomyelin (Spm) or galactocerebroside (GalCer) present in C-26 cells or in the surrounding medium after treatment of C-26 cells with either free, LUV or micelle-containing ¹⁴C radiolabelled C₆ Cer for 2 and 24hr (FIG. 8A) or after treatment with free or LUV containing ¹⁴C radiolabelled C₁₆ Cer for 2 and 48 hr (FIG. 8B). Total lipids were extracted from cells by Bligh and Dyer procedure and the level of radiolabelled C₆Cer was determined by β-counter as described in the Materials and Methods.

FIG. 9A-9B present ¹⁴C radiolabelled C₆Cer and its metabolites on TLC plate visualized by Bio-Imaging analyzer obtained from C-26 cells extracts after treatment for 2, 24 or 48 hr with free or liposomal C₆ Cer: EPC/C₆:; EPC/²k PEG-DSPE/C₆ (FIG. 9A) as well as C-26 cells treated for 2, or 24 hr with micellar (^{2k}PEG-DSPE/C₆) or liposomal (HSPC/C₆ Cer:; HSPC/^{2k}PEG-DSPE/C₆) ¹⁴C radiolabelled C₆ Cer. (FIG. 9B)

FIGS. 10A-10D are confocal laser scanning micrographs demonstrating the exposure of phosphatidylserine (PS) in OV-1063 and C-26 cells treated with IC₅₀ values of liposomal C₆ Cer for 4 hours: Untreated OV-1063 (control, FIG. 10A), treated OV-1063 (FIG. 10B), untreated C-26 cells (control, FIG. 10C) and treated C-26 cells (FIG. 10D).

FIGS. 11A-11D are confocal laser scanning micrographs representing apoptotic changes in the chromatin of OV-1063 (FIGS. 11A and 11B) and C-26 cells (FIGS. 11C and 11D) treated with IC₅₀ values of liposomal C₆ Cer for 16 hours. Untreated OV-1063 and C-26 tumor cells were used as control (FIG. 11A and 11C, respectively).

FIGS. 12A-12D are confocal laser scanning micrographs represents the fragmentation of the DNA in OV-1063 (FIGS. 12A and 12B) and C-26 (FIGS. 12C and 12D) cells treated with IC₅₀ values of liposomal C₆ Cer for 24 hours, while the untreated OV-1063 and C-26 tumor cells were used as control (FIGS. 12A and 12C, respectively).

FIGS. 13A-13B are bar graphs showing a comparison of caspase-3 activity in OV-1063 tumor cells treated for 5 hr with IC₅₀ values of liposomal formulations containing C₂ Cer (C2), C₆ Cer (C6) or C₁₆ Cer (C16) (FIG. 13A) or with free (ethanolic) C₂ Cer, C₆ Cer or C₁₆ Cer (FIG. 13B); Liposomal formulations included EPC:^{2k}PEG-DSPE-ceramide with C₂ Cer, C₆ Cer or C₁₆ Cer. Cells treated with empty liposomes (lacking ceramide) or with ethanol served as controls (FIG. 13A or FIG. 13B, respectively). Following treatment with AC-DEVD-inhibitor is also shown (inhibitor).

FIGS. 14A-14B are bar graphs showing caspase-3 activity in OV-1063 tumor cells treated for 16 hr with IC₅₀ values of either liposomal formulations containing C₂ Cer (C2), C₆ Cer (C6) or C₁₆ Cer (C16) (FIG. 14A) or with free C₂ Cer, C₆ Cer or C₁₆ Cer (FIG. 14B); Liposomal formulations included EPC:^{2k}PEG-DSPE-ceramide with C₂ Cer, C₆ Cer or C₁₆ Cer. Cells treated with empty liposomes or with ethanol served as controls. Following treatment with AC-DEVD-inhibitor is also shown (inhibitor).

FIGS. 15A-15B are graphs showing percent (%) survival of Balb/c mice inoculated i.p. with 1*10⁶ C-26 colon carcinomas and treated as described with sterically-stabilized liposomes comprising EPC or HSPC in combination with ^{2k}PEG-DSPE, and with either C₆ Cer (SSL-C₆ Cer) (FIG. 15A) or with C₄ Cer (FIG. 15B). Untreated mice served as control.

FIG. 16 presents the change in ¹⁴C—C₆Cer/³H-DPPC (¹⁴Cer/³H PL) ratio in mouse plasma at the indicated time points post injection of various lipid assemblies, as described hereinbelow. The initial ceramide/lipid ratio was 0.38.

FIG. 17A-17D present the pharmakokinetics and biodistribution of ¹⁴C-labelled liposomal C6Cer (FIGS. 17A or 17C) or ³H-labelled DPPC (FIGS. 17B or 17D) in plasma and organs of tumor free (FIGS. 17A or 17B)) or tumor bearing (FIGS. 17C or 17D) female Balb/c mice at different time points post-injection of doubly radioactively labeled ¹⁴C C₆ Cer (marker of ceramide) or ³H-DPPC (marker of PC) LUV of the specified lipid composition.

### DETAILED DESCRIPTION OF THE INVENTION

The present invention aims to provide means to deliver lipids which, due to their physicochemical properties, cannot be delivered by themselves or in conventional liposomes. As known in the art, there are many amphiphilic substances that are of biochemical and therapeutic significance and nevertheless, are difficult to be parenterally administrated. Such amphiphilic substances are referred to herein by any one of the terms biologically active lipids or non-liposome forming lipids/substances.

Hithereto, attempts to incorporate non-liposome forming substances into liposomal delivery systems or other ordered lipid assemblies to be delivered to a target site within an individual's body, typically resulted in unstable systems having a tendency to aggregate and/or phase separate, and/or the substances spontaneously leaked out the non-liposomal forming substance upon storage.

Based on the results presented herein, it has now been established that by combining lipids which do not spontaneously form liposomes with an amount of a lipopolymer it is possible to obtain stable (during long term storage at 4° C.) incorporation of such lipids into stable lipid assemblies. These lipid assemblies did not aggregate, do not exhibit a change in their size or in ceramide content during storage for long periods of time (>1 year).

Thus, according to one of its aspects, the present invention provides a stable lipid assembly comprising:
(a) a biologically active lipid having a hydrophobic region and a polar headgroup, wherein the atomic mass ratio between the lipid headgroup and lipid hydrophobic region is less than 0.3;
(b) a lipopolymer having a hydrophobic lipid region and a polymer headgroup wherein the atomic mass ratio between the polymer headgroup and hydrophobic region is at least 1.5.

The lipid assembly of the invention may comprise, in addition to the lipopolymer and biologically active lipid, a lipid matrix. A “lipid matrix” as used herein denotes a liposome forming lipid or a combination of liposome forming lipids forming a lipid lamella, each liposome forming lipid having a packing parameter in the range of 0.74-1. The “liposome-forming lipids” are such that in an aqueous solution they spontaneously form bilayered vesicles (such as liposomes) wherein the hydrophobic region of one monolayer is in contact with the hydrophobic region of the other monolayer, while the polar headgroup moieties are oriented toward the exterior and the interior aqueous phases of the vesicle. Thus, in the presence of liposome forming lipids the lipid assembly of the invention is typically in the form of a liposome.

The lipids forming the lipid matrix typically include one or two hydrophobic acyl chains, or a steroid group, and may contain a chemically reactive group, (such as an amine, acid, ester, aldehyde or alcohol) at the polar head group. Different types of lipid forming the lipid matrix according to the invention are further discussed hereinafter.

The lipid assembly of the invention may be further characterized by the amount of water molecules tightly bound to the polymer headgroup of the lipopolymer. According to the invention, the level of tightly bound water determined, e.g. by DSC and/or by ultrasound, is of at least 60 molecules of water per polymer headgroup as described in Tirosh O. et. al. [Tirosh et al., Biopys. J., 74(3):1371-1379, (1998)].

The biologically active lipid according to the invention is preferably selected from ceramides, ceramines, sphinganines, sphinganine-1-phosphate, di- or tri-alkylshpingosines and their structural analogs, all encompass in the definition provided hereinbefore.

According to one preferred embodiment, the biologically active lipid has the following general formula (I): 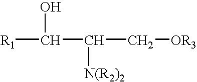
wherein
R₁ represent a C₂-C_{26,} saturated or unsaturated, branched or unbranched, aliphatic chain, the aliphatic chain may be substituted with one or more hydroxyl or cycloalkyl groups and may consist of a cycloalkylene moiety;
R₂ which may be the same or different, represents a hydrogen, a C₁-C₂₆ saturated or unsaturated, branched or unbranched chain selected from aliphatic, aliphatic carbonyl; a cycloalkylene-containing aliphatic chain, the aliphatic chain may be substituted with an aryl, arylalkyl or arylalkenyl group;
R₃ represents a hydrogen, a methyl, ethyl, ethenyl or a phosphate group.

A specific group of biologically active lipids encompassed in the above general definition includes C₂-C₂₆ ceramides (Cer) and more preferably. Some specific ceramides exemplified hereinbelow are C₂ Cer, C₄ Cer, C₆ Cer, C₈ Cer and C₁₆ Cer.

According to yet another embodiment, the biologically active lipid is a dialkylshpingosines. A specific example includes N,N-dimethylsphingosine (DMS).

The above mentioned biologically active lipids have been shown to act, inter alia, as second messengers participating in cell growth and cell differentiation processes as well as in the inhibition of cell proliferation, e.g. by inducing a programmed cell death.

The discovery of pro-apoptotic properties of ceramides and the finding that ceramide inactivates telomerase activity and, therefore, might be cancer-specific made this group of amphiphiles an attractive possibility for antitumor stand alone therapy, as well as in combination with chemotherapeutic agents. Thus, in the following, the formation of a lipid assembly including one or more biologically active lipids was achieved and the biological activity of the resulting lipid assembly was evaluated.

While the majority of the following specific examples concentrate on the incorporation of ceramides into liposomal or micellar structures it is to be understood that other biologically active lipids under the definition provided herein may form part of the lipid assembly of the present invention.

Other biologically active lipids according to the invention include dimethylsphingosine (DMS) mentioned above or trimethylsphingosine (TMS) implicated in inhibition of cell growth [K. Endo et al., Cancer Res. 51:1613-1618, (1991)].

Yet other biologically active lipids according to the invention include diacylglycerols (DAG). DAGs are known to participate in cell signaling as second messengers. This group of biologically active lipid can thus be administrated as part of the lipid assembly of the invention.

Lipopolymers such as those employed by the present invention are known to be effective for forming long-circulating liposomes. Lipopolymers according to the invention comprise preferably lipids, typically, liposome forming lipids, modified at their head with a polymer having a molecular weight equal or above 750 Da. The headgroup may be polar or apolar, however, is preferably a polar head group to which a large (>750 Da) highly hydrated (at least 60 molecules of water per headgroup) flexible polymer is attached. The attachment of the hydrophilic polymer headgroup to the lipid region may be a covalent or non-covalent attachment, however, is preferably via the formation of a covalent bond (optionally via a linker).

Preparation of vesicles composed of liposome-forming lipids and derivatization of such lipids with hydrophilic polymers (thereby forming lipopolymers) has been described, for example by Tirosh et al. [Tirosh et al, Biopys. J., 74(3):1371-1379, (1998)] and in U.S. Pat. Nos. 5,013,556; 5,395,619; 5,817,856; 6,043,094, 6,165,501, incorporated herein by reference and in WO 98/07409. The lipopolymers may be non-ionic lipopolymers (also referred to at times as neutral lipopolymers or uncharged lipopolymers) or lipopolymers having a net negative or a net positive charge.

There are numerous polymers which may be attached to lipids. Polymers typically used as lipid modifiers include, without being limited thereto: polyethylene glycol (PEG), polysialic acid, polylactic (also termed polylactide), polyglycolic acid (also termed polyglycolide), apolylactic-polyglycolic acid, polyvinyl alcohol, polyvinylpyrrolidone, polymethoxazoline, polyethyloxazoline, polyhydroxyethyloxazoline, polyhydroxypropyloxazoline, polyaspartamide, polyhydroxypropyl methacrylamide, polymethacrylamide, polydimethylacrylamide, polyvinylmethylether, polyhydroxyethyl acrylate, derivatized celluloses such as hydroxymethylcellulose or hydroxyethylcellulose. The polymers may be employed as homopolymers or as block or random copolymers.

The most commonly used and commercially available lipids derivatized into lipopolymers are those based on phosphatidyl ethanolamine (PE), usually, distearylphosphatidylethanolamine (DSPE).

A specific family of lipopolymers employed by the invention include PEG-DSPE (with different lengths of PEG chains) in which the PEG polymer is linked to the lipid via a carbamate linkage and Polyethyleneglycol distearoylglycerol. The PEG moiety preferably has a molecular weight of the headgroup is from about 750 Da to about 20,000 Da. More preferably, the molecular weight is from about 750 Da to about 12,000 Da and most preferably between about 1,000 Da to about 5,000 Da. One specific PEG-DSPE employed herein is that wherein PEG has a molecular weight of 2000 Da, designated herein ²⁰⁰⁰PEG-DSPE or ^{2k}PEG-DSPE.

In addition to the contribution of the lipopolymer to the stabilization of the lipid assembly comprising the biologically active lipid, the lipopolymer provide a surface coating of hydrophilic polymer chains on both the inner and outer surfaces of the liposome lipid bilayer membranes. The outermost surface coating of hydrophilic polymer chains is effective to provide the lipid assembly with a long blood circulation lifetime in vivo. In case of liposome formation, the inner coating of hydrophilic polymer chains may extend into the aqueous compartments in the liposomes, between the lipid lamella and into the central core compartment, which may contain additional therapeutic agents.

The lipid matrix according to the invention preferably comprises a physiologically acceptable liposome forming lipid or a combination of physiologically acceptable liposome forming lipids. Liposome-forming lipids are typically those having a glycerol backbone wherein at least one of the hydroxyl groups is substituted with an acyl chain, a phosphate group, a combination or derivatives of same and may contain a chemically reactive group, (such as an amine, acid, ester, aldehyde or alcohol) at the headgroup. Typically, the acyl chain(s) is between 14 to about 24 carbon atoms in length, and has varying degrees of saturation being fully, partially or non-hydrogenated lipids. Further, the lipid matrix may be of natural source, semi-synthetic or fully synthetic lipid, and neutral, negatively or positively charged.

According to one embodiment, the lipid matrix comprises phospholipids. The phospholipids may be a glycerophospholipid. Examples of glycerophospholipid include, without being limited thereto, phosphatidylglycerol (PG) including dimyristoyl phosphatidylglycerol (DMPG); phosphatidylcholine (PC), including egg yolk phosphatidylcholine and dimyristoyl phosphatidylcholine (DMPC); phosphatidic acid (PA), phosphatidylinositol (PI), phosphatidylserine (PS) and sphingomyelin (SM) and derivatives of the same.

Another group of lipid matrix employed according to the invention includes cationic lipids (monocationic or polycationic lipids). Cationic lipids typically consist of a lipophilic moiety, such as a sterol or the same glycerol backbone to which two acyl or two alkyl, or one acyl and one alkyl chain contribute the hydrophobic region of the amphipathic molecule, to form a lipid having an overall net positive charge. Preferably, the headgroup of the lipid carries the positive charge.

Monocationic lipids may include, for example, 1,2-dimyristoyl-3-trimethylammonium propane (DMTAP) 1,2-dioleyloxy-3-(trimethylamino) propane (DOTAP); N-[1-(2,3,-ditetradecyloxy)propyl]-N,N-dimethyl-N-hydroxyethylammonium bromide (DMRIE); N-[1-(2,3,-dioleyloxy)propyl]-N,N-dimethyl-N-hydroxy ethyl-ammonium bromide (DOTIE); N-[1-(2,3-dioleyloxy)propyl]-N,N,N-trimethylammonium chloride (DOTMA); 3β[N-(N′,N′-dimethylaminoethane)carbamoly]cholesterol (DC-Chol); and dimethyl-dioctadecylammonium (DDAB).

Examples of polycationic lipids include a similar lipophilic moiety as with the mono cationic lipids, to which spermine or spermidine is attached. These include, without being limited thereto, N-[2-[[2,5-bis[3-aminopropyl)amino]-1-oxopentyl]amino]ethyl]-N,N-dimethyl-2,3-bis[(1-oxo-9-octadecenyl)oxy]-1-propanaminium (DOSPA), and ceramide carbamoyl spermine (CCS).

The cationic lipids may be used alone in combination with cholesterol, with neutral phospholipids or other known lipid assembly components. In addition, the cationic lipids may form part of a derivatized phospholipids such as the neutral lipid dioleoylphosphatidyl ethanolamine (DOPE) derivatized with polylysine to form a cationic lipopolymer.

The lipid assembly may also include other components typically used in the formation of lipid assemblies (e.g. for stabilization). Examples of such other components includes, without being limited thereto, fatty alcohols, fatty acids, and/or cholesterol esters or any other pharmaceutically acceptable excipients which may affect the surface charge, the membrane fluidity and assist in the incorporation of the biologically active lipid into the lipid assembly. Examples of sterols include cholesterol, cholesteryl hemisuccinate, cholesteryl sulfate, or any other derivatives of cholesterol.

Preferred lipid assemblies according the invention include either those which form a micelle (typically when the assembly is absent from a lipid matrix) or those which form a liposome (typically, when a lipid matrix is present). Lipid assemblies in the form of a liposome may be further characterized by their additive effective packing parameter of the liposomes' constituents, being within the range of 0.74-1.0 [Kumar V V, Proc Natl Acad Sci U S A 88(2):444-448 (1991)]. The term “additive effective packing parameter” refers to the relative (mole % weighted) contribution of the packing parameter of each constituent of the liposome to the average (i.e. the weighted sum) packing parameter of the final lipid composition which constitute the liposome. The fact that the additive effective packing parameter of the structure is within the range of 0.74-1.0 indicates that a liposome is formed and that the combination of all constituents used to form the liposome's lamella results in the formation of stable liposomes.

According to one embodiment of the invention, to achieve a stable lipid assembly, e.g. a stable liposome, the mole percent of the matrix-forming lipid should be in the range between 1% to 34% and preferably in the range of between 1% and 23%.

The lipid assembly may also comprise, associated with the assembly, one or more additional therapeutically active agents. Therapeutically active agents according to the invention may include, without being limited thereto, chemotherapeutic agent or immunomodulators (e.g. immunostimulators). The therapeutically active agents may be loaded in the lipid assembly, e.g. when a liposome or a micelle is formed. The loading of the additional therapeutically active agent may be of any type known in the art, including encapsulation, adhesion, adsorption, entrapment. In the case of liposome it may be located either in the inner or outer wall of the vesicle or in the intraliposomal aqueous phase by passive or remote (active) loading, or it may be embedment in the liposome's membrane. The therapeutic effect achieved by the combination of the biologically active, non-liposome forming lipids and the additional active agent may be additive or synergistic.

The lipid assembly of the invention may also comprise targeting substances associated with the assembly. Targeting substances are known in the art and include, without being limited thereto, antibodies, a functional fragment of an antibody, a cell-surface recognition molecule, etc. the targeting substances may be attached to the liposome by means of a hydrophilic polymer chains or directly to the lipid headgroup. For example, a vesicle-forming lipid may be derivatized with a hydrophilic polymer chain, as described above, and the hydrophilic polymer may be end-functionalized for coupling antibodies to its functionalized end. The functionalized end group may be a hydrazide or hydrazine group which is reactive toward aldehyde groups, although any of a number of PEG-terminal reactive groups for coupling to antibodies may be used. Hydrazides can also be acylated by active esters or carbodiimide-activated carboxyl groups. Acyl azide groups reactive as acylating species can be easily obtained from hydrazides and permit attachment of amino-containing molecules. The functionalized end group may also be 2-pyridyldithio-propionamide, for coupling an antibody or other molecule to the liposome through a disulfide linkage.

The above-described constituents forming the lipid assembly according to the invention can be obtained commercially or prepared according to published methods.

The components of the lipid assembly may be selected to achieve a specified degree of fluidity or rigidity, to control the stability of the assembly during storage as well as after delivery, e.g. in serum and to control the rate of release of the biologically active lipid forming part of the assembly. Lipid assemblies having a more rigid structure, e.g. liposomes in the gel (solid ordered) phase or in a liquid crystalline fluid (liquid disordered) state, are achieved by incorporation of a relatively rigid lipid, for example, a lipid having a relatively high solid ordered to liquid disordered phase transition temperature, such as, above room temperature. Rigid, i.e., saturated, lipids having long acyl chains, contribute to greater membrane rigidity in the assembly. Lipid components, such as cholesterol, are also known to contribute to rigidity in lipid structures especially to reduce free volume thereby reducing permeability. Similarly, high lipid fluidity is achieved by incorporation of a relatively fluid lipid, typically one having a relatively low liquid to liquid-crystalline phase transition temperature, for example, at or below room temperature, more preferably, at or below the target body temperature.

When the lipid assembly is in the form of a liposome, the liposome may be in the form of multilamellar vesicles (MLV), large unilamellar vesicles (LUV), small unilamellar vesicles (SUV) or multivesicular vesicles (MVV) as well as in other bilayered forms known in the art. The size and lamellarity of the liposome will depend on the manner of preparation and the selection of the type of vesicles to be used will depend on the preferred mode of administration. For systemic therapeutic purposes, preferred liposomes are those in the size range of 50-150 nm in diameter (LUV or SUV); for local treatment larger liposomes, such as MLV or MVV, can also be used.

The invention also concerns pharmaceutical compositions comprising an amount of a lipid assembly according to the invention, the amount being sufficient to achieve a biological effect at a target site. The pharmaceutical composition of the invention typically comprises, in addition to the lipid assembly, a physiologically acceptable carrier. The physiologically acceptable carrier employed according to the invention generally refers to inert, non-toxic solid or liquid substances preferably not reacting with the biologically active lipid according to the invention.

The effective amount of the biologically active lipid in the assembly is typically determined in appropriately designed clinical trials (dose range studies) and the person versed in the art will know how to properly conduct such trials in order to determine the effective amount. As generally known, an effective amount depends on a variety of factors including the distribution profile of the lipid assembly within the body, a variety of pharmacological parameters such as half life in the body, undesired side effects, if any, on factors such as age and gender of the treated individual etc. The amount must be effective to achieve a desired therapeutic effect such as improved survival rate or more rapid recovery of the treated subject, or improvement or elimination of symptoms and other indicators associated with the condition under treatment, selected as appropriate measures by those skilled in the art.

The pharmaceutical composition of the invention is administered and dosed taking into account the clinical condition of the individual, the site and method of administration, scheduling of administration, patient age, sex, body weight and other factors known to medical practitioners. The dosage form may be single dosage form or a multiple dosage form to be provided over a period of several days. The schedule of treatment with the lipid assembly of the invention generally has a length proportional to the length of the disease process, the parameters of the individual to be treated (e.g. age and gender) and the effectiveness of the specific biologically active lipid employed.

The lipid assembly can be administered orally, subcutaneously (s.c.) or parenterally including intravenous (i.v.), intraarterial (i.a.), intramuscular (i.m), intraperitoneally (i.p) and intranasal (i.n) administration as well as by infusion techniques.

According to a third of its aspects, the present invention provides a method for the treatment or prevention of a disease, disorder or pathological condition comprising providing an individual in need of said treatment, in a manner so as to achieve a therapeutic effect, an effective amount of a lipid assembly according to the invention.

The treatment according to the invention may be in combination with one or more therapeutically active agents, other than the biologically active lipid of the invention, such as in combination with an immunomodulator, or a chemotherapeutic drug.

According to one embodiment, the therapeutic effect obtained upon delivery of the lipid assembly of the invention comprises inhibition of cell proliferation, e.g. by the induction of cell apoptosis (or any other mechanism of inhibition). Specific examples of lipid assemblies, which may be used for inhibiting cell proliferation are liposomes including in their lamella a therapeutically effective amount of ceramides or DMS.

According to another embodiment, the therapeutic effect obtained by the liposomal structure of the invention is stimulation of cell proliferation and/or differentiation. It was demonstrated that sphingosine-1-phosphate (S1P) and diacylglicerols (DAG) are implicated in cell proliferation [Zhang et al., J. Biol. Chem., 265:76-81, (1990), Zhang et al., J. Cell Biol., 114:155-167, (1991)], and protection from apoptosis [Cuvllier et al., Nature, 381(6585):800-3, (1996)] and therefore may also be delivered to a target site as part of a lipid assembly according to the invention.

### SPECIFIC EXAMPLES

Materials and Methods

Egg phosphatidylcholine (EPC I) and hydrogenated soybean phosphatidylcholine (HSPC) were obtained from Lipoid KG (Ludwigshafen, Germany).

N-carbamyl-poly-(ethylene glycol methyl ether)-1,2-distearoyl-sn-glycero-3-phosphoethanolamine triethyl ammonium salt (^{2k}PEG-DSPE) (the polyethylene glycol moiety having a molecular mass of 2000 Da) was obtained from Genzyme (Liestal, Switzerland) or Nipon Oil and Fats (NOF, Tokyo, Japan).

Polyethyleneglycol distearoylglycerol (^{2k}PEG-DSG-20H) (the polyethylene moiety having a molecular mass of 2000 Da) was obtained from Nipon Oil and Fat (NOF) Corporation (Tokyo, Japan).

N-Acetyl-D-erythro-sphingosine (C₂-Cer), N-tetranoyl-D-erythro-sphingosine (C₄-Cer), N-hexanoyl-D-erythro-sphingosine (C₆-Cer), N-octanoyl-D-erythro-sphingosine(C₈-Cer), and N-palmitoyl-D-erythro-sphingosine (C₁₆-Cer) were obtained from Biolab (Jerusalem, Israel).

tert-Butanol was purchased from BDH, Poole, UK.

Water was purified using WaterPro PS HPLC/Ultrafilter Hybrid model (Labconco, Kansas City, Mo.), providing water with very low levels of total organic carbon and inorganic ions (18.2 mega ohm) in sterile pyrogen-free water.

Physico-Chemical Properties of Ceramides and of Dimethylsphingosine (DMS): Partition in the Two Phases of the Dole System

The distribution of the C₁₈ sphingosine-based ceramides with different acyl chain lengths and of DMS between the polar phase and respectively less polar phase (as defined below) of the Dole two phase system was determined. The two phases are:
1) Heptane-rich hydrophobic upper phase (low dialectric coefficient low polarity phase)
2) Isopropanol-rich hydrophilic lower phase (high dialectric coefficient high polarity phase).

The different ceramides included: short (C₂ Cer and C₄ Cer), medium (C₆ Cer and C₈ Cer), long (C₁₆ Cer). Phase distribution was also determined for various lipids, including EPC, HSPC, ^{2k}PEG-DSPE from which liposomes containing ceramides were later prepared.

In order to determine the distribution of ceramides and DMS between the two phases, DMS and the lipids, 15 μl of 30 mM stock solution of each ceramide in ethanol and 10 μl of 150 mM lipid stock solution in tert-Butanol, respectively, were added and mixed in the Dole system (isopropanol, heptane and pure water in ratio 4:1:0.1, respectively). Then, heptane and DDW were added to the mixture of ceramide in the Dole system in order to reach the ratio of 1:1:1 of isopropanol, heptane and DDW, respectively, followed by centrifugation for 5 min at 2500 rpm to obtain two clear phases [Barenholz, Y and Amsalem, S. In: Liposome Technology 2^{nd} Edn., G Gregoriadis (Ed.) CRC Press, Boca Raton, 1993, vol. 1, pp: 527-616].

The amount of each of the lipids used in each of the two phases was measured by thin-layer chromatography (TLC). For ceramide quantification TLC was developed in a solvent system containing chloroform/methanol (95:5 v/v). For DMS quantification TLC was developed in a solvent system containing chloroform/methanol/ammonia (89/9/2 v/v). Further, the TLC plate was sprayed with Copper sulfate reagent composed of 100 g anhydrous copper sulfate containing 80 ml of phosphoric acid (85%), %) dissolved in 600 ml of DDW. Copper sulphate reagent was applied to the plates by spraying, then heated and lipids appeared as dark brown spot. Silica gel plates 60 F₂₅₄ from Merk (Darsmstadt, Germany) were used. Quantity of ceramides in each phase was calculated from a standard curve of appropriate ceramide. The concentration of the PLs (EPC, HSPC and ^{2k}PEG-DSPE) was determined by lipid phosphorus content (modified Bartlett method) [Barenholz Y, and Amselem, S. (1993)Supra].

The distribution between the two Dole system phases was determined by the equation (as exemplified for ceramide):

$$Distribution ratio = \frac{[ Cer ] up \times [ vol ] up}{[ Cer ] lp \times [ vol ] lp}$$

$$Kp = \frac{[ lipid ] up}{[ lipid ] lp}$$

Where Kp denotes the partition coefficient of a given lipid or amphiphile referred to in the equation as lipid; Cer denotes ceramide; up is an abbreviation for the heptane rich low polarity upper phase and lp is an abbreviation for the polar isopropanol water rich lower phase.

Ceramide-Containing Liposome Preparation

Appropriate amounts of lipid stock solutions were dissolved in tert-butanol and lyophilized. The lyophilized lipids were hydrated with citrate buffer (5 mM, pH 7.0). [Zuidam, N. J. and Barenholz Y. Biochim. Biophys. Acta. 1329(2):211-222 (1997)]. Hydration was performed under continuous vortexing (1 min).

Table 1 summarizes the different lipid compositions used to prepare the aqueous lipid dispersions.

TABLE 1
Lipid dispersion composition (input composition)

| Type of | Ceramide | ^{2k}PEG-DSPE | HSPC |
|---|---|---|---|
| sphingolipid | (mole %) | (mole %) | (mole %) |
| | 12.5 | 5 | 86.5 |
| C₂ Cer | 25 | 5 | 75 |
| | 50 | 5 | 45 |
| | 75 | 5 | 20 |
| | 12.5 | 5 | 86.5 |
| | 25 | 5 | 75 |
| | 50 | 5 | 45 |
| C₆ Cer | 75 | 5 | 20 |
| | 23 | 0 | 77 |
| | 23 | 5 | 72 |
| | 23 | 7.5 | 69.5 |
| | 23 | 10 | 67 |
| | 23 | 12.5 | 64.5 |
| | 23 | 20 | 57 |
| | 12.5 | 5 | 86.5 |
| C₁₆ Cer | 25 | 5 | 75 |
| | 50 | 5 | 45 |
| | 75 | 5 | 20 |
| | 0 | 5 | 95 |

Large unilamellar vesicles (LUV˜100 nm) were prepared by extrusion of MLV 11 times through 0.2-μm- and then 11 times through 0.1-μm-pore-size filters (Poretices, Livermore, Calif. USA) using the extrusion system of Avanti Polar Lipids (Alabaster, Ala.).

Table 2A and Table 2B summarize the different sphingolipid-containing LUV compositions formed.

TABLE 2A
LUV composition (input composition)

| | | ^{2k}PEG- | | |
|---|---|---|---|---|
| Type of | Ceramides | DSPE | HSPC* | EPC* |
| sphingolipid | (mole %) | (mole %) | (mole %) | (mole %) |
| C₂ Cer | 23 | 0 | 77 | 77 |
| | 23 | 7.5 | 69.5 | 69.5 |
| C₄ Cer | 23 | 7.5 | 69.5 | 69.5 |
| | 11.5 | 7.5 | — | 81 |
| C₆ Cer | 23 | 0 | 77 | 77 |
| | 23 | 7.5 | 69.5 | 69.5 |
| | 11.5 | 7.5 | 81 | 81 |
| | 23 | 11 | 66 | 66 |
| | 23 | 5 | 72 | — |
| | 23 | 10 | 67 | — |
| | 23 | 12.5 | 64.5 | — |
| | 23 | 20 | 57 | — |
| C₈ Cer | 23 | 7.5 | 69.5 | 69.5 |
| | 11.5 | 7.5 | — | 81 |
| C₁₆ Cer | 23 | 7.5 | 69.5 | 69.5 |
| | 23 | 0 | 77 | — |
| DMS | 23 | 7.5 | — | 69.5 |
| | 11.5 | 7.5 | — | 69.5 |
| | 0 | 0 | 77 | 77 |
| | 0 | 5 | 72 | 72 |
| | 0 | 7.5 | 69.5 | 69.5 |
| | 0 | 11 | 66 | 66 |
| | 0 | 7.5 | 81 | 81 |

*Liposomes were formed either from HSPC or from EPC, with the indicated mole %

TABLE 2B
LUV composition (input composition)

| | | ^{2k}PEG- | | |
|---|---|---|---|---|
| Type of | Ceramide | DSG-20H | HSPC* | EPC* |
| sphingolipid | (mole %) | (mole %) | (mole %) | (mole %) |
| C₆ Cer | 23 | 5 | 72 | 72 |
| | 23 | 7.5 | 69.5 | 69.5 |
| | 11.5 | 5 | 83.5 | 83.5 |
| | 11.5 | 7.5 | 81 | 81 |
| | 0 | 5 | 72 | 72 |
| | 0 | 5 | 83.5 | 83.5 |
| | 0 | 7.5 | 69.5 | 69.5 |
| | 0 | 7.5 | 81 | 81 |

*Liposomes were formed either from HSPC or from EPC, with the indicated mole %

All formulations were stored at 4° C. The concentration of the PLs (PC and ^{2k}PEG-DSPE) were verified by lipid phosphorus content determination (modified Bartlett method) [Barenholz, Y and Amsalem, S. (1993) Supra.].

Preparation of Radioactive Liposomes

For the preparation of radioactive liposomes appropriate amounts of stock solutions of the desired lipids and ceramides in ethanol were mixed in order to achieve a mole ratio of 81/7.5/11.5 (EPC or HSPC/^{2k}PEG-DSPE/ceramide) or of 88.5/11.5 (EPC or HSPC/ceramide) in liposomes. The desired amount of radioactive lipids, usually 5×10⁶ dpm of [¹⁴C] C₆ Cer or of [¹⁴C]C₁₆ Cer and 15×10⁶ dpm of [³H] dipalmintoylphosphatidylcholine (DPPC) were added. Specific activity of ceramide was 1.2 μCi/μmole and of PC 0.6 μCi/μmole. For comparison, the free C₆ Cer and C₁₆ Cer when used were labeled to reach the same radioactivity with the same amounts of [¹⁴C] C₆Cer and of [¹⁴C]Cl₆Cer.

The lipids were hydrated to form large multilamellar vesicles by adding the lipid solution in ethanol to citrate buffer saline (CBS) (5 mM sodium citrate, 130 mM NaCl, pH 7, 285 mOsmol) in order to achieve the final ethanol concentration of 10% followed by continuous vortexing and sonication for 3 min.

LUV were prepared by extrusion of the above MLV 11 times through 0.4-μm- and then 11 times through 0.1-μm-pore-size filters (Poretics, Livermore, Calif., USA) using the extrusion system Avanti Polar Lipids (Alabaster, Ala.). Then, liposomes were dialyzed against CBS at 4° C. (3 times against 200 volumes of CBS for 30 min and the fourth time overnight against 400 volumes of CBS) to remove the ethanol. For comparison the positively charged MLV composed from DOTAP/DOPE/EPC/C₆ Cer at the ratio of 58.5:29:1:11.5 were labeled with 5×10⁶ dpm of [¹⁴C] C₆ Cer and 15×10⁶ dpm of [³H ] DPPC and hydrated in Hepes buffer (20 mM).

For the preparations of micelles composed from ^{2k}PEG-DSPE and C₆Cer, the appropriate amounts of stock solutions of the ^{2k}PEG-DSPE and C₆Cer were labeled with 5×10⁶ dpm of [¹⁴C] C₆ Cer lyophilized and hydrated with Citrate buffer (5 mM, pH 7.0). Hydration was performed under continuous vortexing. Specific activity of ceramide was 1.2 μCi/μmole.

The lipid composition of the radioactive liposomes used in cell culture uptake studies are described below in the Table 2C, while the radioactive liposomal compositions used in the in vivo studies are described below in the Table 2D.

TABLE 2C
Radioactive assemblies comprising ceramide C₆Cer or C₁₆Cer

| | | | ceramide ¹⁴C specific | | DPPC ³H specific | |
|---|---|---|---|---|---|---|
| | | | radioactivity | | radioactivity | |
| | Assembly | Size | | Recovery | | Recovery |
| Lipid assembly composition | type | (nm) | dpm/μmole | (%) | dpm/μmole | (%) |
| EPC/^{2k}PEG-DSPE/C₆Cer | LUV | 88 ± 5 | 2.2 × 10⁶ | 92 | 1.19 × 10⁶ | 90 |
| (81.5:7.5:11.5) | | | | | | |
| EPC/C₆Cer (88.5:11.5) | LUV | 112 ± 8 | 1.94 × 10⁶ | 73 | 1.1 × 10⁶ | 83 |
| HSPC/^{2k}PEG-DSPE/C₆Cer | LUV | 76 ± 4 | 1.77 × 10⁶ | 74 | 0.98 × 10⁶ | 81.5 |
| (81.5:7.5:11.5) | | | | | | |
| HSPC/C₆Cer | LUV | 109 ± 10 | 1.7 × 10⁶ | 71 | 0.84 × 10⁶ | 70 |
| (88.5:11.5) | | | | | | |
| ^{2k}PEG-DSPE/C₆Cer | micelles | 20 ± 4 | 2.3 × 10⁶ | 96 | — | — |
| (65:35) | | | | | | |
| EPC/^{2k}PEG-DSPE/C₁₆Cer | LUV | 87 ± 6 | 1.98 × 10⁶ | 75 | 1.25 × 10⁶ | 95 |
| DOTAP/DOPE/EPC/C₆Cer | MLV | 600 ± 200 | 2.4 × 10⁶ | 100 | 0.99 × 10⁶ | 75 |

TABLE 2D
Radioactive assemblies comprising ceramide (C₆Cer)

| | | | ¹⁴C₆ specific | | DPPC ³H specific | |
|---|---|---|---|---|---|---|
| | | | radioactivity | | radioactivity | |
| | Assembly | | | Recovery | | Recovery |
| Lipid assembly composition | type | Size (nm) | dpm/μmole | (%) | dpm/μmole | (%) |
| EPC/^{2K}PEG-DSPE/C₆Cer | LUV | 90 ± 7 | 9.4 × 10⁵ | 78 | 0.41 × 10⁶ | 84 |
| (81/7.5/11.5) | | | | | | |
| EPC/C₆Cer (88.5/11.5) | LUV | 115 ± 10 | 9.7 × 10⁵ | 80 | 0.35 × 10⁶ | 71 |
| HSPC/^{2K}PEG-DSPE/C₆Cer | LUV | 87 ± 4 | 1.04 × 10⁶ | 87 | 0.49 × 10⁶ | 100 |
| (81/7.5/11.5) | | | | | | |
| HSPC/C₆Cer (88.5/11.5) | LUV | 120 ± 6 | 1 × 10⁶ | 83 | 0.38 × 10⁶ | 77 |

Lipid Assembly Characterization

Large (˜100 nm) unilamellar liposomes were prepared from mixtures of each of the various ceramides (different chain lengths: short (C₂ Cer and C₄ Cer), medium (C₆ Cer and C₈ Cer), and long (C₁₆ Cer), or of dimethylsphingosine (DMS), a bilayer-forming PL (EPC or HSPC), and the lipopolymer ^{2k}PEG-DSPE or ^{2k}PEG-DSG.

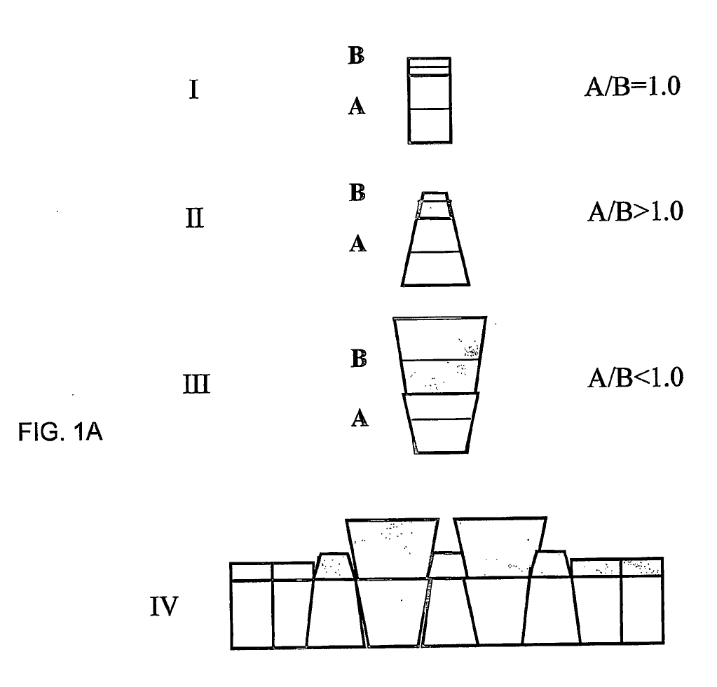

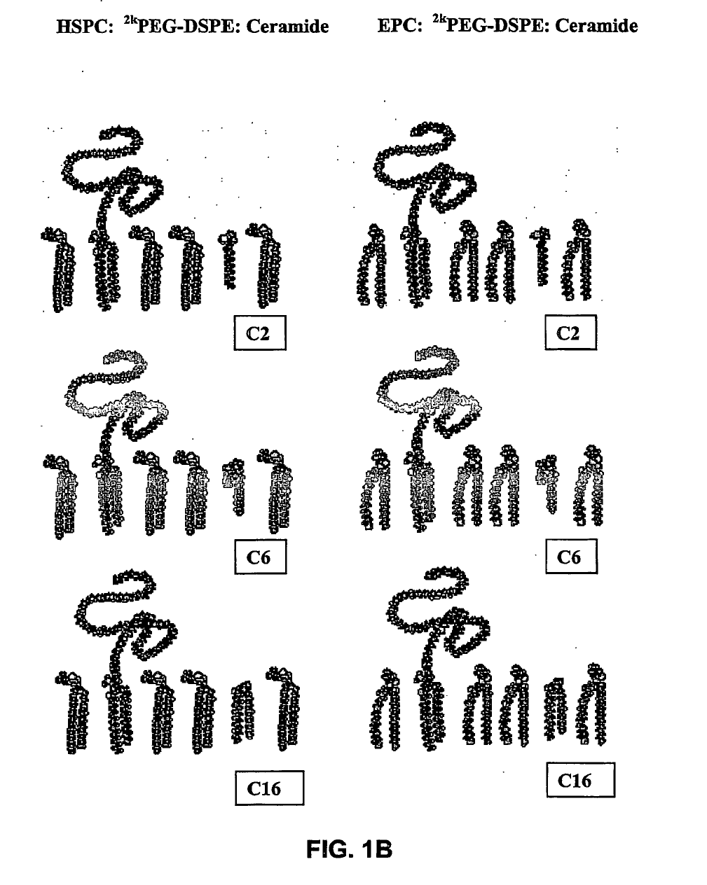

The geometric shapes of the various components of the liposomes prepared are illustrated in FIG. 1A and 1B. FIG. 1A schematically illustrates the shapes of different molecules employed according to the invention: (I) the cylindrical shape of liposome forming lipids, e.g. EPC/HPC, having a packing parameter (A/B) in the range of 0.8-1.0. A specific example of a lipid of group (I) includes a lipid with a glycerol backbone with two ester-linked fatty acids and a phosphocholine head (e.g. hydrogenated soybean phosphatidylcholine (HSPC) with PP=0.816 [Garbusenko O, Barenholz Y and Priev A, submitted] that is fully saturated, very enriched with disteroyl PC (83%), the backbone also containing 10% of palmithoyl and 2-steroyl PC (Lipoid, certificate of analysis) or Egg phosphatidylcholine (EPC) with PP=0.802 [Garbusenko O, Barenholz Y and Priev A. Packing parameter of PEGylated lipid bilayer., submitted] that has one saturated (position 1) and one unsaturated (position 2) acyl chains (mainly composed from palmitoyl (32%), oleyl (32%),and stearic acid (23%) composition presented in [Samuni A. M, et al. Free Radic Biol Med., 23(7):972-9 (1997); (1I) an inverted, truncated, cone shape of biologically active lipids (e.g. ceramide), having a packing parameter (A/B) greater than 1; (III) lipids having a cone shape having a packing parameter (A/B) less than 1 due to the very large headgroup (lipopolymers, e.g. a PEGylated lipid in which the polyethylene glycol headgroup is attached to amino-group of distearoyl phosphatidyl ethanolamine (PEG-DSPE), and has a packing parameter of 0.487); and (IV) a schematic illustration of a lipid assembly comprising the above components. FIG. 1B schematically illustrates more specific examples of lipid assemblies according to the invention including the following alternatives: ceramide (C₂, C₆ or C₁₆ Cer) ^{2K}PEG-DSPE in combination with either HSPC or EPC. This illustration exhibits the bulky headgroup of the lipopolymer in combination with the truncated inverted cone shape of the biologically active lipid (ceramide).

The working hypothesis is that lipopolymers, due to their very large head-group and drying effect in the bilayer head-group region, should increase the level of biologically active lipid incorporation in the liposomes and improve the liposome's stability and slow down rate of loss (desorption) of these substances to other hydrophobic environments present in the system, such as cell membranes, lipoproteins, or liposomes not containing the non-liposome-forming lipids. The lipid assemblies were evaluated for their capacity to include active amphiphiles and for the difference in their input to output lipid composition (biologically active lipid mole %). More specifically, the role of the lipid composition and especially of the mole % lipopolymer (such as ^{2k}PEG-DSPE and ^{2k}PEG-DSG), in lipid assembly bilayer capacity to include sphingolipids (one specific group of biologically active lipids according to the invention). The effect on the assembly stability upon storage, rate of loss of non-liposome forming biologically active lipid, toxicity, and therapeutic efficacy were also evaluated.

Particle Size Distribution Measurements

The particle size distribution of all liposome dispersions prepared was determined at 25° C. by dynamic light-scattering (DLS) using the ALV-NIBS/HPPS ALV-Laser, Vertriebsgesellschaft GmbH, (Langen, Germany) instrument.

Measurement of Biologically Active Lipid Content in Different Lipid Assemblies (Liposome Formulations and Lipid Dispersions)

The amount of biologically active (typically non-liposome forming lipid) amphiphiles in lipid dispersions or in liposomes (LUV) was measured by thin-layer chromatography (TLC). Ceramide or DMS were resolved from other lipids using solvent system of chloroform/methanol (95:5 v/v) and of chloroform/methanol/ammonia (89/9/2 v/v), respectively. Ceramide or DMS were detected by Copper sulfate reagent. Copper sulphate was applied to the plates by spraying, then heated and ceramide or DMS appeared as black spots. Silica gel plates 60 F₂₅₄ from Merk (Darsmstadt, Germany) were used. Migration and quantity of ceramides or DMS was calculated in comparison to a standard curve of the appropriate molecule. Lipid quantification was performed using Fluor-S MultiImager.

Measurement of Maximal Level of Biologically Active Lipid Loading into MLV and LUV

Lipid dispersions (MLV) with different mole % of C₆ Cer were centrifuged at 10,000 rpm for 10 min. The pellet (MLV and other aggregates) and supernatant (LUV, SUV and micelles) fractions were collected and analyzed for ceramide/PL mole ratio (from which ceramide mole % was calculated) by TLC. The above pellets of dispersions having different mole % of C₆ Cer was downsized by repeated extrusion and centrifuged at 10,000 rpm for 10 min. The pellet (residual “MLV”) and the supernatant referred to as LUV (which include LUV, SUV and micelles) fractions were collected and analyzed for ceramide/PL mole ratio by Silica gel plates 60 F₂₅₄ TLC. Ceramide and ^{2k}PEG-DSPE were resolved from EPC by a solvent system containing chloroform/methanol/water (90:15:2.5 v/v) and detected. Migration and quantity of ceramides and ^{2k}PEG-DSPE were calculated based on the calibration curve of appropriate ceramide, PC and of ^{2k}PEG-DSPE standards as described in the Materials and Methods.

Characterization of Liposomal Thermotropic Behavior

The thermothropic behavior of HSPC bilayers with different mole % of ceramides and lipopolymers were studied using both differential scanning calorimetry (DSC) and differential turbidity (determined as optical density measurements).

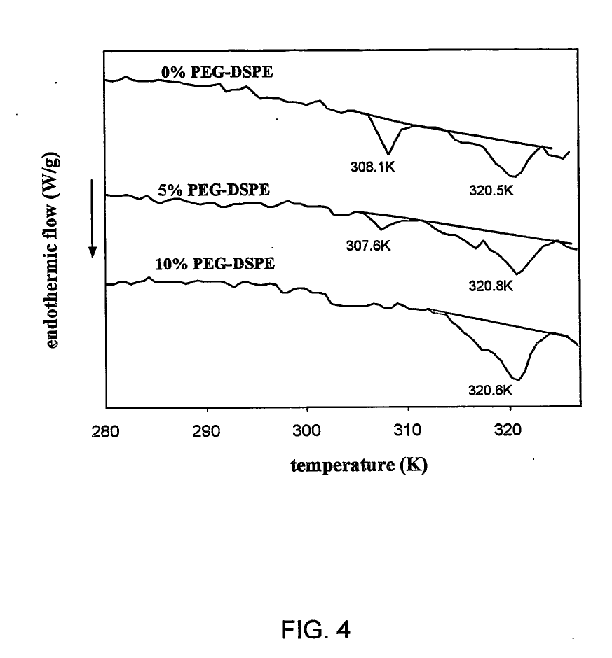

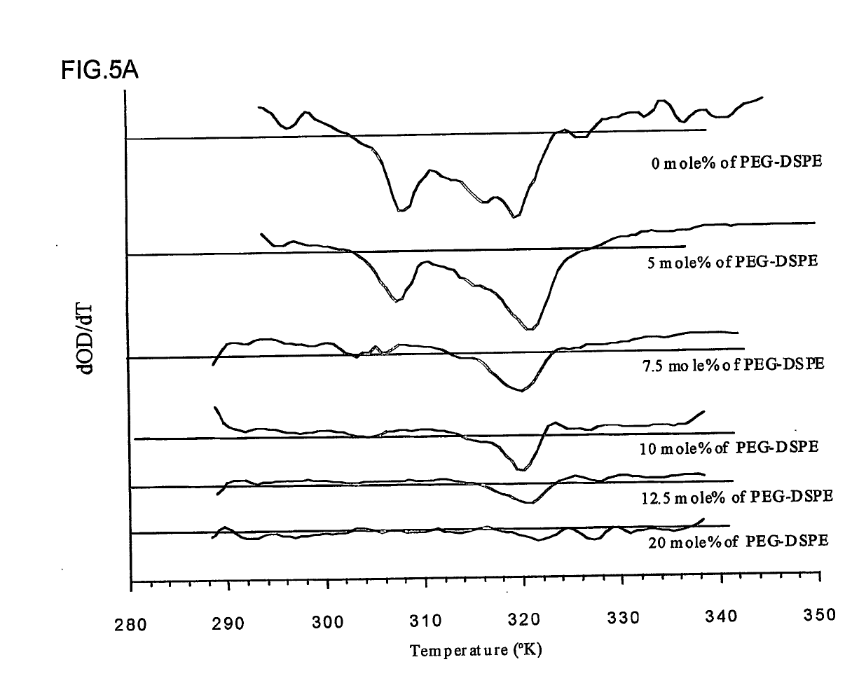

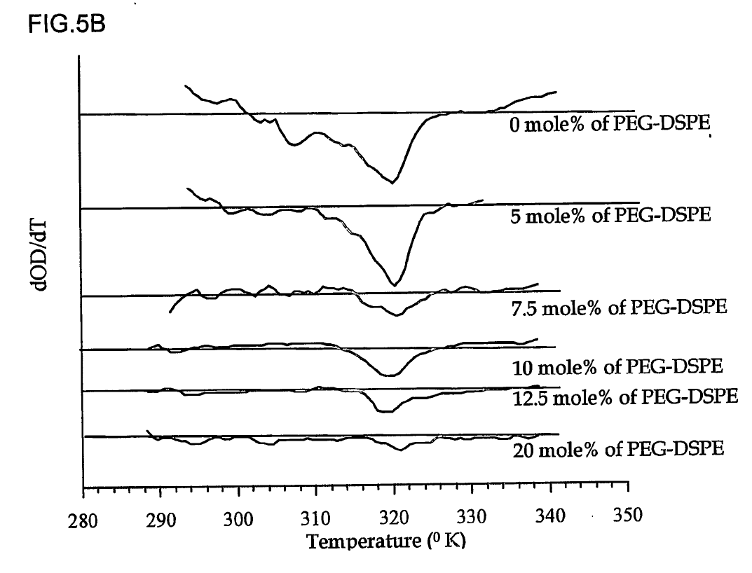

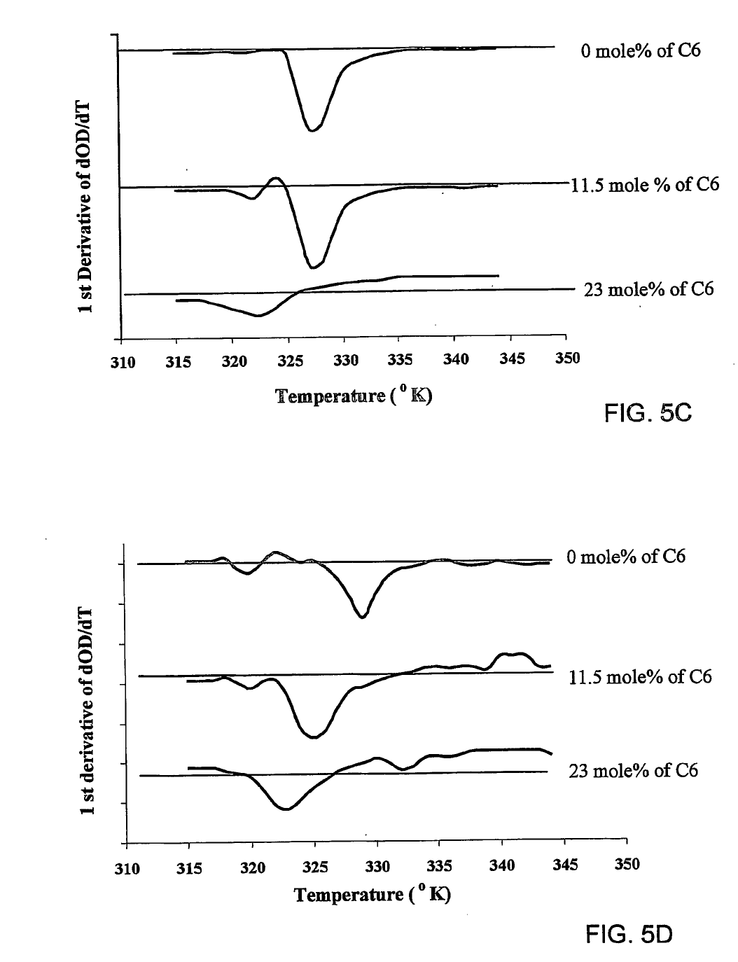

DSC measurements were performed on MLV using Mettler thermal analyzer model 4000. Scans were recorded at 10 K/min until a stable spectrum was obtained, followed by a scan at 2° K/min over a temperature range of 50° K. Parameters obtained from DSC measurements include the temperature range of solid ordered to liquid disordered (gel to liquid crystalline) phase transition, the temperature of maximum change in heat capacity (Tm) and the enthalpy change (ΔH) of the phase transition. Temperature-dependent changes in specific turbidity (OD/mg lipid) were determined using a Carry 300 Bio UV-visible double beam spectrophotometer (arian, Australia). The change in O.D. during temperature scanning relates to the differences in bilayer packing and can be used to monitor solid ordered and liquid-disordered phase transition of the bilayer as was demonstrated in the past [Barenholz and Amsalem, Supra 1993] and confirmed by the studies presented here (compare FIGS. 4 and 5A). These measurements give similar results to the DSC data. Scans of O.D. at 300 nm were carried over a temperature range of at least 50° K at rate of 2° K/min or lower. Scans were analyzed in two ways: 1(a) OD as a function of temperature, 2 (b) d(OD)/dT—as a function of temperature. Tm of the phase transition was also determined as the temperature of maximum change in the sample specific turbidity (determined as OD/mg lipid) during temperature scanning. Furthermore the DSC and spectrophotometer scans were analyzed for the symmetry and the width at the half height of the phase transition peak [R. L. Biltonen and D. Lichtenberg. Chemistry and physics of lipids. 64(1-3):129-142 (1993)].

HSPC:^{2k}PEG-DSPE (95:5) MLV with different mole % of ceramides and both MLV and LUV of HSPC:C₆Cer (3:1) with different mole % of ^{2k}PEG-DSPE were also measured for the temperature-dependent changes in specific turbidity (OD/mg lipid) by a Carry 300 Bio UV-visible double beam spectrophotometer (arian, Australia). The change in O.D. during temperature scanning relates to the differences in bilayer packing and can be used to monitor solid ordered and liquid-disordered phase transition of the bilayer as was demonstrated in the past (ref) [Barenholz and Amsalem, (1993) Supra.] and confirmed by the work presented here (compare FIGS. 4 and 5). The second way gives similar results to the DSC data. Scans of O.D. at 300 nm were carried over a temperature range of at least 50° K at rate of 2° K/min or lower. Scans were analyzed in two ways: 1(a) OD as a function of temperature, 2 (b) d(OD)/dT—as a function of temperature.

Volumetric Measurements

The density (ρ) of suspensions at a selected liposome concentration (c) (g/ml) was determined using the vibrating tube densitometer DMA-60/DMA-601 (Anton Paar, Austria) with a precision of ±3×10⁻⁶ g/mL. The partial specific volume V of the diluted suspensions:
V=1/ρ₀−lim[(ρ_{c-ρ}₀)/(ρ₀c)]
where ρ₀ is the density at zero liposome concentration (solvent density). All LUV suspensions were dialyzed and degassed in vacuum for at least 1 h before performing volumetric measurements.

Temperature control by a water bath had an accuracy of ±0.01° C. The procedure was performed at 25° C.

Ultrasonic Measurements

Ultrasonic velocity of different LUV formulation was measured in order to calculate the adiabatic compressibility of the liposomes. Measurements of ultrasonic velocity was made using the “resonator method” analogous to the method described by Eggers and Funck [F. Eggers and Funk. Rev. Sci. Instrum. 44:969-977 (1973)].

Compressibility Calculation

The adiabatic compressibility, K, which is defined by (dV/dP) where P is pressure applied at constant entropy was obtained from the measurements of density ρ and the sound velocity U:
K=β₀(2V−2lim[(U_{c}−U₀)/(U₀c)]−1/ρ₀)

For each determination of K, three independent measurements of U and V were carried out. The value for the density and sound velocity of water at different temperatures was taken from Kell [Kell, G. S. J. Chem. Eng. Data. 20:7-108 (1975)].

Stability of Lipid Assemblies Comprising Biologically Active Lipids

Chemical stability of the lipid assemblies was examined by one or more of the following parameters:

a) Measurement of dispersion pH (pH meter)

b) PL acylester hydrolysis by determination of change in non-esterified (free) fatty acids (NEFA) released upon PL hydrolysis [Barenholz et. al. From Liposomes: a practical approach, 2^{nd} Edn., RRC New ed, IRL Press Oxford, 1997] or by TLC [Y. Barenholz, and S. Amselem., (1993) Supra].

Physical stability of the lipid assemblies was examined by one or more of the following parameters:

a) assembly size distribution by dynamic light-scattering (DLS).

b) Level of free (non liposome/aggregated) biologically active lipid (ceramide or DMS) by TLC which is based on determining of the biologically active lipid (ceramide or DMS)/PL mole ratio in the pellet (free ceramide/DMS) and in the supernatant (assembled ceramide or DMS, which are part of the lipid assembly).

Determination of LUV Interactions with Serum

Different types of LUV (as described above) were incubated for 10 min with FCS (Biological Industries Beit-HaEmek, Israel) at 25% and 50% (by volume) of FCS, respectively, and LUV-serum interaction was determined by the following different methods:
a. Measurement of changes in liposome particle size distribution by dynamic light scattering method at 25° C. using ALV-NIBS/HPPS ALV-Laser, Vertriebsgesellschaft GmbH, (Langen, Germany).
b. Measurement of turbidity ratio (TR) of LUV by spectrophotometer according to the following formula:

For particles having size 1/20 of the wavelength or smaller (λ) (ray light scattering) it was expected that

$$\frac{{OD}_{1}}{{OD}_{2}} = {( \frac{{λ}_{2}}{{λ}_{1}} )}^{4}$$

$$TR = {OD}_{1} / {OD}_{2} = {( {λ}_{2} / {λ}_{1} )}^{4}$$

However, for a heterogeneous particle population which include larger particles, the TR=OD₁/OD₂ is expected to be smaller than λ₁/λ₂⁴ [Barenholz and Amsalem, (1993) Supra.].

Determination of turbidity ratio as absorbance ratio was done at λ₁=300 and λ₂=600 nm. Under such conditions TR of 16 is expected.

TR is the ratio of turbidity at 300 nm to turbidity at 600 nm, OD is optical density (using OD₁=at 300 nm and OD₂=at 600 nm).

In vitro Toxicity and Efficacy Studies

Cell Cultures

Several tumor cell lines in monolayers were used. A human ovarian carcinoma cell line (OV-1063), established at the Hadassah University Hospital, a human colon carcinoma cell line (C-26), a DOX-sensitive M-109S (human breast carcinoma), and a DOX-resistant, M-109R (human breast carcinoma). All cell lines were maintained in RPMI-1640 medium supplemented with 10% FCS, antibiotics, and glutamine. All culture medium components were purchased from Biological Industries (Beit-HaEmek, Israel). Both cell lines were maintained at 37° C. in a water-jacketed CO₂ incubator.

Treatment of Cells in Culture

Two methods were used for evaluating the effect of the biologically active lipids on the cancer cell cultures:

a) Various ceramides (Cer) with different chain lengths: short (C₂ Cer and C₄ Cer), medium (C₆ Cer and C₈ Cer), and long (C₁₆ Cer) were first dissolved in 100% ethanol to a concentration of 30 mM, added to a serum-containing medium and mixed immediately. The final ethanol concentration in the serum-containing culture medium ranged from 0.1-0.5% (depending on the type of ceramide (Cer)).

b) Various ceramides with different chain lengths: short C₂ Cer, medium C₆ Cer, and long C₁₆ Cer were dissolved in ethanol:dodecane (98:2 v/v) to a concentration of 30 mM and added to serum-free medium and mixed. Final concentrations of the ethanol and dodecane in the culture medium containing 10% of FCS ranged from 0.098-0.49% and 0.002-0.01%, respectively, depending on the type of ceramide.

Methylene Blue Assay of Cell Survival

The cytotoxicity of the examined assemblies comprising ceramide was tested by the methylene blue (NM) staining assay [Gorodetsky, R. et al. Int. J. Cancer. 75:635-642 (1998)]. A known number of exponentially growing cells in 200 μL of medium were plated in 96-microwell, flat-bottomed plates. For each of the variants tested, 4 wells were used. Following 24 hr of incubation in culture, 20 μL of different concentrations of the examined assemblies were added to each well containing untreated cells.

The following controls were used: Citrate buffer (5 mM, pH 7); Ethanol solution in medium RPMI 1640 (final concentration of 0.1%); A solution RPMI 1640 of ethanol:dodecane (98:2 w/w) at a final concentration of 0.1% and 0.2%, respectively.

Cells were exposed to assemblies for 4, 24, 72 or 96 hr. At the end of assembly exposure, for a fixed time interval, the drug-treated cells, as well as parallel control cells, were washed, and the incubation continued in fresh medium until termination of the experiment. Following 72 hr or 96 hr of growth, cells were fixed by adding 50 μL of 2.5% glutaraldehyde to each well for 15 min. Fixed cells were rinsed 10 times with deionized water and once with borate buffer (0.1 M, pH 8.5), dried, and stained with MB (100 μL of 1% solution in 0.1 M borate buffer, pH 8.5) for 1 h at room temperature (r.t.). Stained cells were rinsed thoroughly with de-ionized water to remove any non-cell-bound dye and then dried. The MB bound to the fixed cells was extracted by incubation at 37° C. with 200 μL of 0.1 N HCl for 1 h, and the net optical density of the dye in each well was determined by a plate spectrophotometer (Labsystems Multyskan Bichromatic, Finland) at 620 nm.

Preparation of Lipid Cell Extracts for Determination of Ceramide and Lipid Assembly Cell Uptake Studies

C-26 colon carcinoma cells were seeded into six-well plates at density of 2.5×10⁵ in 2 ml of complete RPMI-1640 medium supplemented with 10% FCS, antibiotics, and glutamine. The cells were allowed to grow for 48 hr and replaced with 1 ml of complete serum containing medium. Liposomal or free radiolabelled ceramides were added to the C-26 cells in order to get the final ceramide concentration of 20 μM (7×10⁴ dpm/ml of ¹⁴C C₆ Cer or C₁₆ Cer and 2×10⁵ dpm/ml of ³H-DPPC) and incubated for 2, 24 and 48 hr at 37° C. The radioactive doses of ceramides and lipid (dpm/μmole) are described in Table 2C. After these predefined time periods cells were tripsinized and washed twice with PBS. Cell lipids, lipids of the medium and lipids of the wash fraction were extracted by the Bligh and Dyer procedure [E. G. Bligh and W. J. Dyer, Can. J. Biochem. Physiol. 37:9111-9117 (1959)]. Briefly, chloroform, methanol, and DDW were added to the cells at a final ratio of 1:2:1 (by vol.), and incubated for 10 min at 45° C. Then the mixtures were centrifuged at 140,000 rpm for 5 min. The supernatants containing the lipids were taken and chloroform and DDW were added in order to reach the final volume ratio of chloroform: methanol:DDW of 1:1:1. Two phases were formed and well separated after centrifugation. The water/methanol rich upper phase was removed while the chloroform-rich lower phase which contains >99% of the lipids was washed once with synthetic upper phase composed of chloroform:methanol:DDW (6:94:96, by volume). The lipid-containing lower phase was dried under nitrogen stream and redissolved in chloroform: methanol (2:1, by vol.) ready for analysis.

The lipid mixture was applied to silica gel TLC plates and developed in the solvent system of chloroform:methanol: DDW (84:16:1,5, by vol.) and detected by Copper sulfate reagent. The TLC plates were photographed by the Fluor-S-Multyimiger (Bio-Rad, Hercules, Calif.). Migration of lipids from cells extracts, medium, and wash fractions was visualized in comparison to a different well established commercial markers. The retention factor RF (defined as the distance traveled by the compound divided by the distance traveled by the solvent) of various molecules are as follows: SPM-0.04, EPC or HSPC-0.1, DOTAP: 0.24, GalCer: 0.29-0.38, GluCer: 0.4, C6 Cer: 0.68, and C16 Cer: −0.88.

The TLC plates were then subjected overnight to imaging plate and the radioactivity was measured by Bio-Imaging analyzer (FUJI BAS 1000, Japan) then the radioactive bands were scraped from the TLC plate, placed into the test tube containing scintillation medium Opti-Fluor (Packard Bioscience, Groningen, Netherlands) and the radioactivity was counted by a β-counter.

Assessment of Apoptosis

Apoptosis (programmed cell death) was assessed in treated tumor cell lines by several methods:

Early events in apoptosis were assessed by staining of the C-26 and OV-1063 cells with Merocyanine 540 (MC 540) and 4′,6-diamidino-2-phenylindone dihydrochloride (DAPI), both from Molecular Probe, Eugene, Oreg. This assay is based on the observation that soon after the initiation of apoptosis, phosphatidylserine (PS) trans-locates from the inner face of the plasma membrane to the cell surface. At this point, PS can be detected readily by staining with MC 540, which has a strong affinity to PS [Reid, S et al. J. Immunol. Methods 192(1-2):43-54 (1996)] Changes in chromatin was assessed by staining with DAPI, which preferentially stains double-stranded DNA.

In the experiments presented herein samples containing 5×10⁵ cells were cultured on 6-well plates covered with a glass coverslip. After treatment of the cells with IC₅₀ concentrations of the drugs, cells were washed with PBS and incubated for 2 min in the dark in 500 μL of PBS containing 2.5 μL of MC 540 (1 mg/ml). Subsequently, cells were washed with PBS, fixated with 4% formaldehyde and stained with 300 μL⁻ DAPI (3 μM). Thereafter, a glass coverslip was placed on a glass slide, which was then photographed using a confocal laser scanning microscope (CLMS) (Zeiss 410, Germany), a high-resolution microscope that allows viewing and quantification of fluorescence at the different cell compartments.

Late steps in apoptosis involve changes in the structure of chromatin and DNA. Two methods to follow-up and quantify these changes were used:

a) The morphology of chromatin was assessed by staining with Hoechst-33342 obtained from Calbiochem (La Jolla, Calif.), a molecule which when reside in the minor groove of the DNA strand enhance its fluorescence intensity and, therefore, it is preferentially stains dsDNA [Jouvet, P. Mol. Biol. Cell 11:1919-1932 (2000)]. Briefly, samples containing 5*10⁵ cells were cultured on 6-well plates covered with a glass coverslip. After treatment of cells with IC₅₀ of drugs, cells were washed with PBS and fixated with 4% formaldehyde. After that cells were stained with Hoechst-33342 (5 μg/ml) and washed. Thereafter, glass coverslip was placed on a glass slide and photographed using a CLMS.

b) The DNA fragmentation was measured by terminal deoxynucleotide transferase (TdT) mediated deoxyuridine triphosphate (dUTP) nick-end labeling (TUNEL) assay (Apoptosis detection system, Fluorescein, Promega, Madison Wis., USA) [Gavrieli Y. et.al. J Cell Biol. 119:493 (1992)]. This method takes advantage of massive DNA fragmentation during apoptosis and generation of many free 3′ OH termini, which may be labeled by fluorescent nucleotides that enzymatically added to the DNA by TdT. Briefly, OV-1063 cells (3*10⁴ cells/ml) and C-26 cells (1.2*10⁴ cells/ml) were cultured in Lab-Tek chambered coverglass system (Nagle Nunc, Naperville, Ill.) for 48 hr. After that, cells were treated with IC₅₀ concentrations of the drugs for 24 hr and cellular apoptosis was detected by this kit according to the manufacture instructions and measured by CLMS.

Biochemically, apoptosis was verified by the EnzChek™ Caspase-3 Assay Kit (Molecular Probes). This allows the detection of apoptosis by assaying for increases in caspase-3 and other DEVD-specific protease activities (e.g., caspase-7). The basis for the assay is rhodamine 110 bis-(N-CBZ-aspartyl-L-glutamyl-L-valyl-aspartic acid amide) referred to as Z-DEVD-R110. This substrate is a bisamide derivative of rhodamine 110 (R110) containing DEVD peptides covalently linked to each of R110's amino groups. Upon enzymatic cleavage, the nonfluorescent bisamide substrate is converted to the fluorescent R110, which was quantified by a fluorescence microplate reader (Tecan) using excitation at 485±10 nm and emission at 535±10 nm. Briefly, C-26 and OV-1063 cells were treated with IC₅₀ of lipid assemblies comprising ceramide formulations or free ceramides for 5 or 16 hr. The AC-DEVD-inhibitor was used to confirm that the observed fluorescence signal in treated samples is due to the activity of caspase-3 protease. Both induced and control cells were then harvested and lysed. Enzyme reactions were performed in 96-well plates with 50 μg of cytosolic proteins (55 min. of incubation) and a final concentration of 25 μM Z-DEVD-R110 substrate, as described in the kit protocol.

In vivo Evaluation of Toxicity and Antitumor Efficacy of Assembled Ceramide

All the experimental procedures which make use of animals (mice and dogs) were performed in accordance with the standards required by the Institutional Animal Care and Use Committee of the Hebrew University and Hadassah Medical Organization and approved by the Committee. Acute and chronic toxicity of ceramides with different chain lengths: short (C₂ Cer) and (C₄ Cer) medium (C₆ Cer) and (C₈ Cer) and long (CI₆ Cer) encapsulated into sterically stabilized liposomes (SSL) was checked on 8 week-old female BALB/C mice and compared to SSL without ceramide. In addition, the various ceramides encapsulated into liposomes consisting from EPC or HSPC and ^{2k}PEG-DSPE were evaluated for their in vivo toxicity and anti-tumor efficacy. These liposomal formulations at ceramide and lipid concentration of 2 μmole/mouse and 6 μmole/mouse, respectively, were injected i.v. three times at 3-day intervals and mice weight changes and survival were followed.

To test therapeutic efficacy, female BALB/C mice (in the weight range of 16-20 g) were injected i.p. with 1*10⁶ C-26 colon carcinomas. The viability of these cells was >90% by trypan blue exclusion. The therapeutic efficacy of SSL comprising C₆ Cer and C₄ Cer at ceramide and lipid concentration of 1-2 μmole/mouse and 6 μmole/mouse, respectively, was studied. SSL-C₆ Cer (EPC or HSPC LUV stabilized by ^{2k}PEG-DSPE and containing C₆ Cer) treatment began at day 3 post tumor injection and was repeated twice for a total of three injections at three days intervals. SSL-C₄ Cer (EPC LUV stabilized by ^{2k}PEG-DSPE and containing C₄ Cer) was injected 3 days later after tumor injection at dose of 2 μmole per mouse and was repeated one weak and 10 days later at dose of 1 μmole per mouse The median survival and percentage increase in life span of treated (T) over control (C) animals (T×100/C)−100 were calculated.

Biodistribution Studies in Tumor Free and Tumor-Bearing Mice

Eight to 10-week-old BALB/c female mice, obtained through the Animal Breeding House of the Hebrew University (Jerusalem, Israel), were housed at Hadassah Medical Center at a specific pathogen free (SPF) faculty with food and water ad libitum. Radioactive liposomes containing C₆ Cer (1 μmole)/mouse and phospholipid (6 μmole)/mouse were injected i.v. At 2 min, 10 min, 30 min, 3.5 hr, and 24 hr h after injection, the animals were anesthetized with 4% chloralhydrate (Fluka, USA), bled by eye enucleation, and immediately sacrificed for removal of liver, spleen, kidney, and intestine. Each time point consisted of 2 mice. Plasma was separated by centrifugation at 3,000 rpm for 5 min.

In the case of tumor bearing mice, each mouse was injected with one inoculum of tumor cells (1×10⁶ C-26 cells) subcutaneously into the left flank. 9 days later radioactive liposomes containing C₆ Cer (1 μmole)/mouse and phospholipid (6 μmole)/mouse were injected i.v. At 3.5 hr and 24 hr h after injection, the animals were anesthetized with 4% chloralhydrate bled by eye enucleation, and immediately sacrificed for removal of liver, spleen, kidney, lung and tumor.

¹⁴C₆Cer and ³H DPPC Measurements in Plasma and Organs

From the samples prepared as described above, 100 μl of plasma samples and various organs were processed using a Sample Oxidizer (Model 307, Packard Instrument Co., Meridien, Conn.) left overnight in a dark, cool place and measured by β-counting (KONTRON Liquid Scintillation Counter).

Statistical Analysis

Survival times were recorded for a total of 35 days after treatment. Median survival times and the statistical significance of differences in survival curves were calculated by means of the log-rank test using Prism Software (GraphPad, San Diego, Calif.). Differences were considered significant at P<0.05.

Results

Physico-Chemical Properties of the Biologically Active Lipids

All lipids used in these studies including the various ceramides, DMS, HSPC, EPC and ^{2K}PEG-DSPE, were characterized for their distribution between heptane rich low dialectric coefficient medium and isopropanol/water rich high dialectric coefficient medium using the Dole extraction procedure (which includes calculated Dole polar upper phase/Dole polar lower phase) and for heptane rich/isopropanol and water rich partition coefficient (Kp, Barenholz Y. and Amselem S., Supra 1993) as described in Materials and Methods. The results are presented in Table 3. As shown, 79% of C₂Cer were found in the polar isopropanol and water-rich phase; while 21% of the C₂Cer were found in non-polar heptane-rich upper phase. The results also show that increase in the length of the ceramide acyl chain increased the distribution into the heptane rich phase (21, 32, 63, 72 and 89% of C₂Cer, C₄Cer, C₆Cer, C₈Cer and C₁₆Cer, respectively). Similarly, 57% of the DMS were found in non-polar heptane-rich upper phase. For the liposome forming PCs, ˜85% and 64% of EPC and HSPC respectively distributed into the isopropanol rich phase compared with 100% of the ^{2K}PEG-DSPE.

It was found that Kp of EPC is smaller than that of HSPC (0.35 and 1.13, respectively). The difference in the physico-chemical properties of EPC and HSPC may be due to presence of the cis double bonds in the EPC molecule which reduce hydrophobic surface area and therefore reduces overall hydrophobicity relative to HSPC. One hundred percent (100%) of the ^{2k}PEG-DSPE distributed into the more polar isopropanol and water rich phase.

TABLE 3
Phase distribution and Kp values

| | Type of | % of lipid in | % of lipid in | | |
|---|---|---|---|---|---|
| | biologically | heptane-rich | isopropanol/water | | Liposome |
| Nₒ | active lipid | upper phase | lower phase | Kₚ | formation^{a} |
| 1 | C₂Cer | 21.2 | 78.8 | 0.54 | No |
| 2 | C₄Cer | 31.5 | 68.5 | 0.92 | No |
| 3 | C₆Cer | 62.7 | 37.3 | 3.17 | No |
| 4 | C₈Cer | 72 | 28 | 5.25 | No |
| 5 | C₁₆Cer | 89 | 11 | 8.21 | No |
| 6 | DMS | 57 | 43 | 2.58 | No |
| 7 | EPC | 15.2 | 84.8 | 0.35 | Yes |
| 8 | HSPC | 36 | 64 | 1.13 | Yes |
| 9 | ^{2k}PEG-DSPE | 0 | 100 | 0 | No |

^{a}being a liposome forming lipid by itself

The Kp results demonstrate that among all lipids used in this study the lipopolymer ^{2K}PEG-DSPE is the most polar in agreement with being the only lipid used which self-assembled as micelles. Among the ceramides used the longer is the N-acyl moiety the higher is the Kp. Surprisingly, C₂ Cer and C₄ Cer have lower Kp than HSPC. All other ceramides have higher Kp than the two PCs. In order to determined the state of aggregation of the C₂ Cer and C₄Cer in aqueous phase, two PCs were used: the saturated predominantly C18:0 HSPC and the unsaturated EPC. The large difference in the degree of saturation was translated into differences in exposed hydrophobic area, which for unsaturated EPC is smaller than for saturated HSPC. This explains why Kp for EPC is lower than Kp of HSPC.

The critical aggregation concentration (CAC) of the ceramides is the concentration at which aggregation of monomers to an amphiphile assembly occurs. CAC of the C₂, C₆, and C₁₆ ceramides was determined in filtered pure water containing 0.3% ethanol by measuring at room temperature surface tension as a function of ceramide concentration. The measurements were done using μThrougs Kibron System (Helsinki, Finland) which determine the surface tension at the air/water interface. The measurement at each concentration was repeated until a constant value of surface tension was reached [Zuidam, N. and Barenholz, Y., Supra (1997)]

The following CAC values were obtained from our measurements:

| C₂Cer | C₆Cer | C₁₆Cer |
|---|---|---|
| 10⁻⁶M | 10⁻⁴-10⁻⁵M | 10⁻⁹-10⁻¹⁰M |

Namely, concentration of monomers (and possibly other small meres like dimers) of C₂ and C₆ ceramide in the aqueous medium was much higher (almost million times) than of C₁₆ ceramide, and at equal concentration the level of monomers of C₂ and even more for C₆ ceramide is expected to be higher than of C₁₆ ceramide. In addition, direct release of C₆Cer, but not of C₁₆Cer from liposomes composed from EPC:^{2k}PEG-DSPE:Cer (81:7.5:11.5 mole %)was determined using the same μThrougs Kibron System by following the changes of surface tension with time of liposome incubation at 37° C.

Surface-Pressure Area Isotherms

The tested ceramides (C₂ Cer, C₄ Cer, C₆ Cer, C₈ Cer, C₁₆ Cer) were dried in vacuum overnight, weighted and dissolved in hexane/isopropanol (3:2 vol.) to make the following stock solutions.

| | | Area/molecule |
|---|---|---|
| Ceramide | (mM) | (A²) |
| C₂ | 2.94 | 38 |
| C₄ | 2.72 | 46 |
| C₆ | 2.52 | 50 |
| C₈ | 2.36 | 45.5 |
| C₁₆ | 1.86 | 37.5 |

The surface-pressure/area isotherms were obtained on pure water sub-phase (similar isotherms were obtained on 140 mM NaCl). Barrier speed during compression was 20 nm/min for all monolayers. The C₂ Cer, C₄ Cer, C₆ Cer, C₈ Cer had a clear collapse point at pressure about 42 mN/m while the C₁₆ Cer raised slowly up to 50 mN/m having less defined collapse points.

The C₂ Cer did not give a stable monolayer, the C₄ Cer monolayer was almost as stable as all the others, the long chain ceramide C₁₆ Cer also gave an unstable monolayer (as demonstrated by having a substantially continuous collapse).

The minimal area per molecule of different ceramides (C₂ Cer, C₄ Cer, C₆ Cer, C₈ Cer, C₁₆ Cer) was calculated at the constant pressure of 20 mN/m. It was found that C₆ Cer has the largest area per molecule of about 50 Å². The area per molecule of C₂ Cer, C₄ Cer, C₈ Cer, C₁₆ Cer was about 38, 46, 45.5, and 37.5², respectively.

Percent of “Loading” of the Various Ceramides onto MLV and LUV

The % loading (association) of various ceramides and DMS in MLV and LUV of various lipid assemblies was determined.

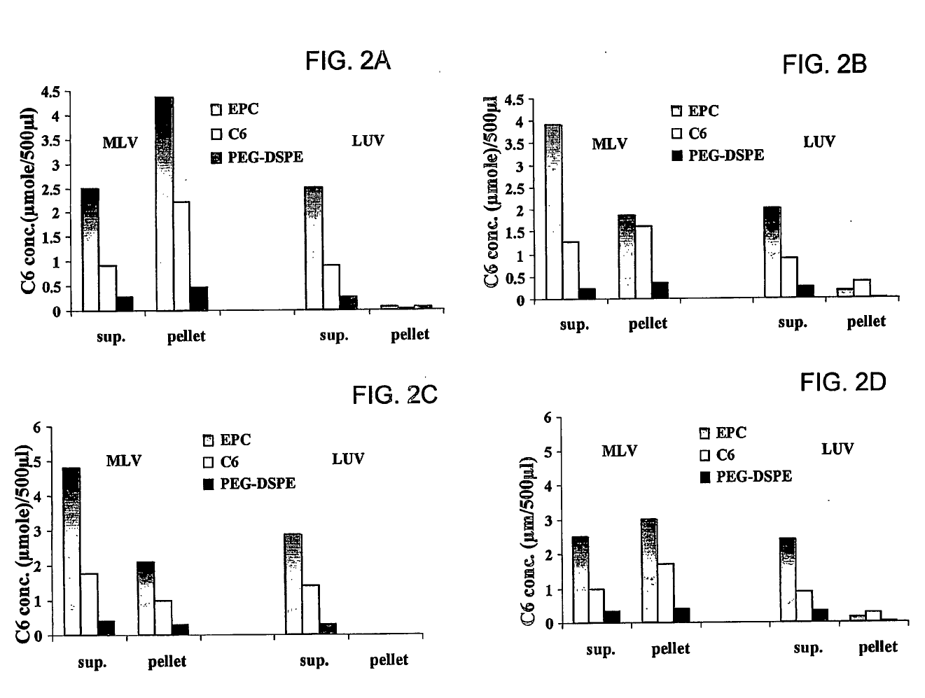

MLV and LUV comprising C₆ ceramide, EPC and ^{2k}PEG-DSPE at different ratios: 58.5:34:7.5; 54.5:38:7.5; 56:34:10; 52:38:10; were prepared as described in the Materials and Methods. Aliquots of supernatant and pellets obtained after centrifugation of the liposomes and analysis by silic acid TLC using chloroform/methanol/water 90:15:2.5 as solvent system (which separate well between the three liposomal components and enables their quantification) were obtained. FIGS. 2A-2D exhibit the level of incorporation of the ceramide into the different formulations, respectively, determined as described in the Materials and Methods.

Additional results are presented in Table 4 showing that 60%-95% of the 11-23 mole % ceramide used for liposome preparation (input composition) can be incorporated into the LUV membrane (output ratio), depending on input mole % of ceramide, on the liposome PC and the mole % ^{2K}PEG-DSPE (Table 4). For example, in liposome assemblies with a higher (7.7:1) PL-ceramide ratio (e.g. EPC or HSPC:^{2K}PEG-DSPE:C₆Cer (81:5:11.5)) the loading of C₆Cer was higher by about 14% than for lipid assemblies with lower (3.3:1) lipid-ceramide ratio (EPC/HSPC:^{2K}PEG-DSPE:C₆Cer (69.5:7.5:23)).

Further it was established that higher mole % of ^{2k}PEG-DSPE in the assemblies results in a higher % of ceramide loading (Table 4). The loading of C₆ Cer onto assemblies comprising the neutral lipopolymer ^{2k}PEG-DSG was significantly high (Table 4). It was also observed that % of loading of C₆ Cer was slightly greater in assemblies composed of HSPC than in assemblies composed of EPC (Table 4).

TABLE 4
LUV formulations

| | | | Physical | | ceramide | | Output of | Physical | |
|---|---|---|---|---|---|---|---|---|---|
| | | | state of the | Input of | in | % of | ceramide | stability | |
| | | Size | membrane | ceramide | liposome | recovered | or DMS | follow-up | |
| No. | Liposome composition (mole ratio) | (nm) | at 37° | (mole %) | % of input | PL | (mole %) | at 4° C. | |
| 1 | EPC (100) | 89 | LD | | | 94 | | 24 | M |
| 2 | HSPC (100) | 92 | SO | | | 75 | | 24 | M |
| 3 | EPC:^{2k}PEG-DSG (93.5:6.5) | 102 | LD | | | 114 | | 24 | M ongoing |
| 4 | HSPC:^{2k}PEG-DSG (93.5:6.5) | 100 | SO | | | 126 | | 24 | M ongoing |
| 5 | EPC:^{2k}PEG-DSPE (90:10) | 111 | LD | | | 88 | | 24 | M ongoing |
| 6 | HSPC:^{2k}PEG-DSPE (90:10) | 112 | SO | | | 85 | | 24 | M ongoing |
| 7 | EPC:^{2k}PEG-DSG (91.5:8.5) | 74 | LD | | | 108 | | 24 | M ongoing |
| 8 | HSPC:^{2k}PEG-DSG (91.5:8.5) | 96 | SO | | | 106 | | 24 | M ongoing |
| 9 | EPC:^{2k}PEG-DSPE (86:14) | 85 | LD | | | 104 | | 24 | M ongoing |
| 10 | HSPC:^{2k}PEG-DSPE (86:14) | 82 | SO | | | 104 | | 24 | M ongoing |
| 11 | EPC:^{2k}PEG-DSPE (91.5:8.5) | 104 | LD | | | 96 | | 24 | M ongoing |
| 12 | HSPC:^{2k}PEG-DSPE (91.5:8.5) | 84 | SO | | | 90 | | 24 | M ongoing |
| 13 | EPC:^{2k}PEG-DSPE:C₂Cer (69.5:7.5:23) | 97 | LD | 23 | 65 | 93 | 16.5 | 24 | M |
| 14 | HSPC:^{2k}PEG-DSPE:C₂Cer (69.5:7.5:23) | 82 | SO | 23 | 58 | 83 | 16.5 | 4 | M |
| 15 | EPC:^{2k}PEG-DSPE:C₄Cer (69.5:7.5:23) | 91 | LD | 23 | 57 | 96 | 14 | 14 | M |
| 16 | HSPC:^{2k}PEG-DSPE:C₄Cer (69.5:7.5:23) | 75 | SO | 23 | 55 | 90 | 14.5 | 3 | M |
| 17 | EPC:^{2k}PEG-DSPE:C₄Cer (81:7.5:11.5) | 88 | LD | 11.5 | not done | not done | not done | 18 | M |
| 18 | EPC:Cer₆(77:23) | 98 | LD | 23 | 59 | 100 | 14 | 3.5 | M |
| 19 | EPC:^{2k}PEG-DSPE:C₆Cer (69.5:7.5:23) | 98 | LD | 23 | 68 | 80 | 19.5 | 4.5 | M |
| 20 | EPC:^{2k}PEG-DSPE:C₆Cer (66:11:23) | 98 | LD | 23 | 95 | 100 | 22 | 6 | M |
| 21 | HSPC:C₆Cer (77:23) | 147 | SO | 23 | 61 | 94 | 15 | 5 | W |
| 22 | HSPC:^{2k}PEG-DSPE:C₆Cer (69.5:7.5:23) | 99 | SO | 23 | 74 | not done | not done | 2 | M |
| 23 | HSPC:^{2k}PEG-DSPE:C₆Cer (66:11:23) | 153 | SO | 23 | 74 | 104 | 18 | 6 | M |
| 24 | EPC:^{2k}PEG-DSPE:C₆Cer (81:7.5:11.5) | 104 | LD | 11.5 | 78 | 98 | 9.2 | 24 | M |
| 25 | HSPC:^{2k}PEG-DSPE:C₆Cer (81:7.5:11.5) | 84 | SO | 11.5 | 71 | 84 | 10 | 8 | M |
| 26 | EPC:^{2k}PEG-DSPE:Chol:C₆Cer | 92 | LO | 11.5 | 75 | 82 | 9.6 | 1 | W |
| | (44:7.5:37:11.5) | | | | | | | | |
| 27 | EPC:^{2k}PEG-DSG:C₆Cer (72:5:23) | 81 | LD | 23 | 63 | 112 | 13 | 1 | W |
| 28 | HSPC:^{2k}PEG-DSG:C₆Cer (72:5:23) | 86 | SO | 23 | 127 | 113 | 25.7 | 1 | W |
| 29 | EPC:^{2k}PEG-DSG:C₆Cer (69.5:7.5:23) | 88 | LD | 23 | 109 | 113 | 22 | 2 | W |
| 30 | HSPC:^{2k}PEG-DSG:C₆Cer (69.5:7.5:23) | 91 | SO | 23 | 109 | 113 | 22 | 1 | W |
| 31 | EPC:^{2k}PEG-DSG:C₆Cer (83.5:5:11.5) | 81 | LD | 11.5 | 103 | 112 | 10.6 | 24 | M ongoing |
| 32 | HSPC:^{2k}PEG-DSG:C₆Cer (83.5:5:11.5) | 94 | SO | 11.5 | 102 | 81 | 15.3 | 24 | M ongoing |
| 33 | EPC:^{2k}PEG-DSG:C₆Cer (81:7.5:11.5) | 85 | LD | 11.5 | 100 | 111 | 10.4 | 24 | M ongoing |
| 34 | HSPC:^{2k}PEG-DSG:C₆Cer (81:7.5:11.5) | 92 | SO | 11.5 | 113 | 114 | 11.3 | 24 | M ongoing |
| 35 | EPC:^{2k}PEG-DSPE:C₈Cer (69.5:7.5:23) | 85 | LD | 23 | 62 | 100 | 14.5 | 24 | M ongoing |
| 36 | HSPC:^{2k}PEG-DSPE:C₈Cer (69.5:7.5:23) | 86 | SO | 23 | 71 | 96 | 17 | 24 | M ongoing |
| 37 | EPC:^{2k}PEG-DSPE:C₈Cer (81:7.5:11.5) | 79 | LD | 11.5 | not done | not done | not done | 24 | M ongoing |
| 38 | EPC:^{2k}PEG-DSPE:C₁₆Cer (69.5:7.5:23) | 93 | LD | 23 | 65 | 107 | 14 | 24 | M ongoing |
| 39 | HSPC:^{2k}PEG-DSPE:C₁₆Cer (69.5:7.5:23) | 127 | SO | 23 | 56 | 72 | 18 | 24 | M ongoing |
| 40 | EPC:^{2k}PEG-DSPE:DMS (69.5:7.5:23) | 67 | SO | 23 | 75 | 101 | 17 | 24 | M ongoing |
| 41 | EPC:^{2k}PEG-DSPE:DMS (81:7.5:11.5) | 98 | SO | 11.5 | 90 | 100 | 10.5 | 24 | M ongoing |

LD, liquid disordered (fluid phase);
LO liquid ordered (fluid phase);
SO, solid ordered (gel phase);
% of ceramide in LUV was determined by TLC
Output of ceramide in LUV was calculated in accordance to % of PL recovery, which was measured by determination of organic phosphorus (modified Bartlet method) (ref: Shmeeda et al., 2003; Barenholz and Amselem, 1993)
M = months;
W = weeks

The Ratio Between Input and Output Mole % of Various biologically Active Lipids in LUV

The ratio between input (mole % of all lipids used for preparation of lipid assembly formulations, in this particular case LUV)) and output (mole % of the lipid used found in the LUV) of the various lipids in the liposomes was determined as the input to output ratio for all lipids as sphingolipid to PL mole ratio in the isolated LUV. It was found that the output mole % (recovery) of ceramide in LUV was medium to high (60%-95%), depending on % of PL recovery and the mole % lipopolymer in LUW (Table 4). The higher the mole % lipopolymer, the higher was the ceramide output mole % and recovery.

The Role of Lipopolymers in Assembly Capacity to Load the Biologically Active, Non-Liposomne Forming Lipids

The role of lipopolymers such as ^{2k}PEG-DSPE on assembly capacity to include (in their lipidic layer) non-liposome forming biologically active lipids and on the lateral distribution of the biologically active lipids was also studied. As shown in Table 4, it was found that increasing the mole % of the lipopolymer in the LUV lipid bilayer increased the level of ceramide (e.g. C₆ Cer) saturation in the LUV lipid bilayer as well as increasing LUV stability upon storage at 4° C.

Maximum Loading Capacity of Biologically Active Lipids onto MLV and LUV

The maximum loading capacity of ceramide C₆ Cer into liposomes (multilamellar vesicles (MLV) and large unilamellar vesicles (LUV≦100 nm)) was determined. Liposomes composed from EPC:^{2k}PEG-DSPE:C₆ Cer with different mole ratios were employed.

Maximal loading capacity for C₆ Cer was determined by measuring levels of PL and ceramide in the liposomes 24 hr post-liposome preparation. Specifically, the PL/ceramide ratio was calculated as described in the Materials and Methods and in the following Scheme 1. It was found that the maximal loading capacity of C₆ Cer into the liposomes was between 34 mole % to 38 mole % (Table 5A-5B, and FIG. 2).

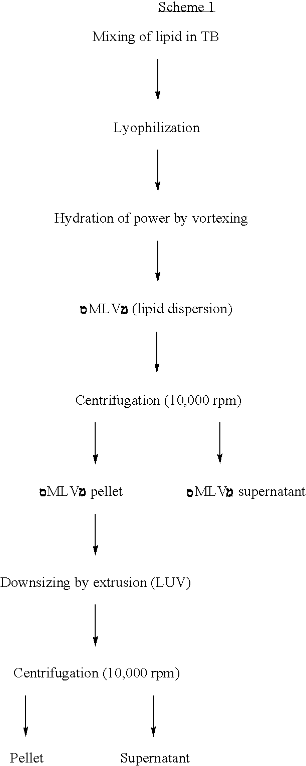

TABLE 5A
MLV

| | | Concentration of | Concentration of ^{2k}PEG- | Concentration of | ceramide/PL mole |
|---|---|---|---|---|---|
| | Initial ceramide/ | EPC in liposomes | DSPE in liposomes mM | C₆Cer in | ratio in liposomes |
| Liposome formulation | lipid mole ratio | mM (% from total | (% from total PEG- | liposomes mM (% | (mole % of |
| (mole ratio) | in liposomes | PL) | DSPE) | from total C₆Cer) | ceramide) |
| EPC:^{2k}PEG-DSPE:C₆Cer | | | | | |
| (58.5:7.5:34) | | | | | |
| supernatant | 0.515 | 5 (37%) | 0.56 (38%) | 1.86 (27%) | 0.384 (25.4) |
| Pellet | | 8.7 (66%) | 0.94 (63%) | 4.4 (65%) | 0.513 (34) |
| EPC:^{2k}PEG-DSPE:C₆Cer | | | | | |
| (54.5:7.5:38) | | | | | |
| Supernatant | 0.613 | 7.8 (63%) | 0.45 (30%) | 2.54 (33%) | 0.286 (18) |
| Pellet | | 4.3 (35%) | 0.7 (46%) | 3.2 (53%) | 1.111 (63) |
| EPC:^{2k}PEG-DSPE:C₆Cer | | | | | |
| (56:10:34) | | | | | |
| supernatant | 0.515 | 9.6 (72%) | 0.82 (41%) | 3.56 (52%) | 0.385 (25.4) |
| Pellet | | 4.2 (32%) | 0.6 (31%) | 2 (30%) | 0.476 (31.4) |
| EPC:^{2k}PEG-DSPE:C₆Cer | | | | | |
| (52:10:38) | | | | | |
| supernatant | 0.666 | 5 (41%) | 0.7 (35%) | 2 (25%) | 0.4 (26.4) |
| Pellet | | 6 (38%) | 0.8 (40%) | 3.4 (54%) | 0.862 (53) |

TABLE 5B
LUV

| | Initial | | Concentration of | | ceramide/PL |
|---|---|---|---|---|---|
| | ceramide/ | Concentration of | ^{2k}PEG-DSPE in | Concentration of | mole ratio in |
| Liposome | lipid mole | EPC in liposomes | liposomes mM | ceramide (C₆Cer) in | liposomes |
| formulation (mole | ratio in | mM | (% from total | liposomes mM | mole % of |
| ratio) | liposomes | (% from total PL) | ^{2k}PEG-DSPE) | (% from total C₆) | ceramide) |
| EPC:^{2k}PEG- | | | | | |
| DSPE:C₆Cer | | | | | |
| (58.5:7.5:34) | | | | | |
| Supernatant | 0.515 | 5 (37%) | 0.54 (36%) | 1.8 (26%) | 0.370 (24) |
| Pellet | | 0.14 (1%) | 0.112 (1%) | 0.06 (0.8%) | 0.417 (27.5) |
| EPC:^{2k}PEG- | | | | | |
| DSPE:C₆Cer | | | | | |
| (54.5:7.5:38) | | | | | |
| Supernatant | 0.625 | 4 (33%) | 0.48 (33%) | 1.76 (23%) | 0.455 (28) |
| Pellet | | 0.32 (2.6%) | 0.022 (2%) | 0.76 (10%) | 2.5 (155) |
| EPC:^{2k}PEG- | | | | | |
| DSPE:C₆Cer | | | | | |
| (56:10:34) | | | | | |
| Supernatant | 0.515 | 0.515 | 0.56 (29%) | 2.8 (38%) | 0.5 (33) |
| Pellet | | No pellet | | | |
| EPC:^{2k}PEG- | | | | | |
| DSPE:C₆Cer | | | | | |
| (52:10:38) | | | | | |
| supernatant | 0.666 | 4.8 (40%) | 0.694 (35%) | 1.78 (22%) | 0.385 (24) |
| Pellet | | 0.29 (1.8%) | 0.056 (3%) | 0.58 (7%) | 2 (124) |

Concentration of C₆Cer in MLV LUV was measured by determination of organic phosphorus (modified Bartlet method);
Concentration of ^{2k}PEG-DSPE in MLV and LUV was determined using TLC;
Concentration of C₆Cer in MLV and LUV was determined using by TLC.
Measurements were done 24 h post-liposome preparation.

It was found that in the pellet of MLV having 34 mole % of ceramide, the ceramide/PL ratio was conserved, however, in the pellet of MLV having 38 mole % of ceramide, the ceramide/PL ratio was higher i.e., the pellet was enriched in ceramide (Table 5A-5B, FIG. 2). Also, it was found that pellet of LUV prepared from a lipid mixture containing 38 mole % ceramide was enriched with ceramide (Table 5A-5B, FIG. 2). It was found herein that using a lipid phospholipids composition of EPC/^{2k}PEG-DSPE/C₆ Cer the ceramide was loaded up to a level of 34 mole % while at 38% mole Cer the LUV were unstable. Thus, a mole % of ceramide of less than 38 was preferred.

Further ^{2k}PEG-DSPE affected the mole % of ceramide in the liposome lipid bilayer. The ceramide/PL ratio was lower in pellet of MLV having 10 mole % of ^{2k}PEG-DSPE as compared to the pellet of MLV having 7.5 mole % of ^{2k}PEG-DSPE (1:1.16 compared to 1:0.9, both consisting of 38% of ceramide), which consequences with a pellet enriched in ceramide (Table 5A-5B). This suggests that there may be an upper mole % of ^{2k}PEG-DSPE limit for achieving an effective ceramide loading.

Characterization of Thermotropic Behavior of Lipid Dispersions Consisting of HSPC, ^{2k}PEG-DSPE and Different Ceramides

Thermotropic Behavior of Ceramide/HSPC Lipid Assemblies

The effect of ceramide with different chain lengths on the thermotropic behavior of HSPC bilayers was determined by use of increasing amount of ceramide in lipid dispersions (MLV) consisting of HSPC and 5 mole % of ^{2k}PEG-DSPE.

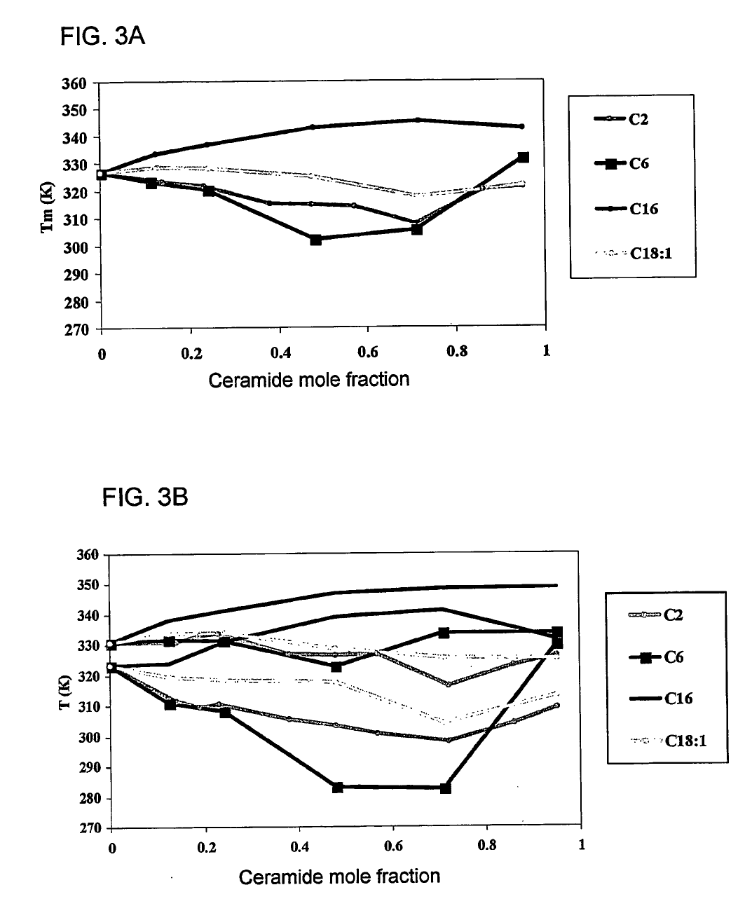

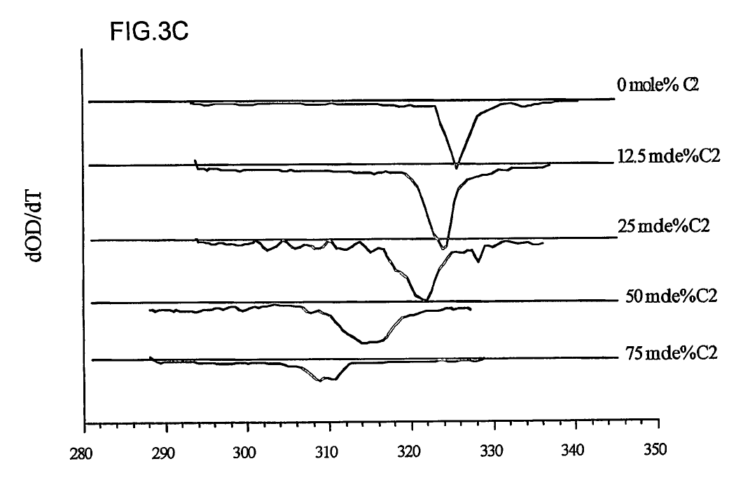

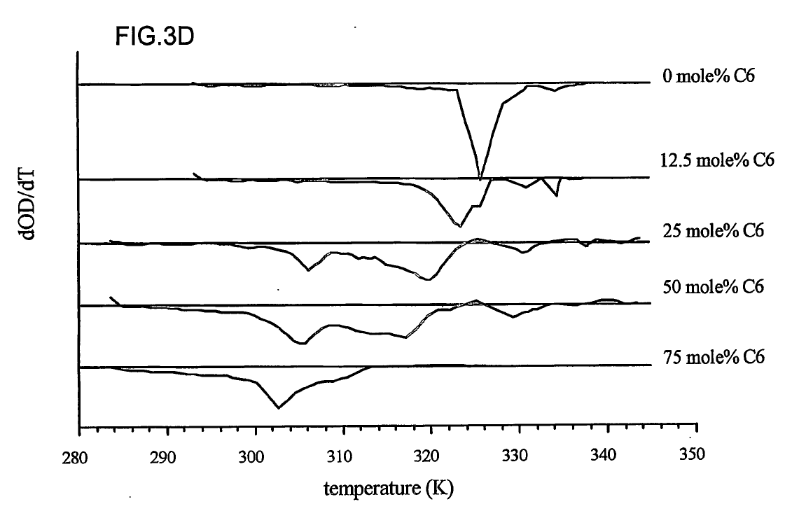

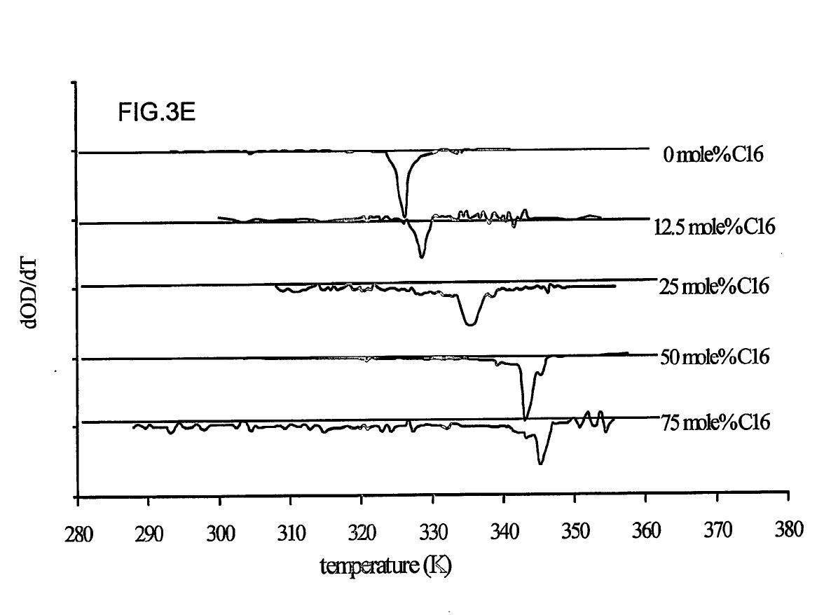

The lipid dispersions showed an asymmetric peak (endotherm) with a tailing to high T with a Tm of 326.8 K. FIG. 3A and 3B show the effect of increasing amounts of ceramide with different chain lengths on both Tm (FIG. 3A and onset and offset of MLV phase transition (FIG. 3B, as determined by DSC thermograms).

With the increasing mole % of C₂ Cer in the MLV, Tm lowered, the peak became broader and a tailing occurred at lower T (FIG. 3A). The addition of C₆ Cer had a similar effect on the endotherm, but the tailing to low T was much more prominent. A second transition peak was clearly visible at a C₆ Cer concentration of 25 mole % at T=308° K.

FIG. 3B shows that C₁₆ Cer and C_{18:1} Cer have a better miscibility with HSPC than C₆ Cer which has the worst miscibility with HSPC and the broadest phase transition range. When comparing the different ceramides the miscibilities the order is: C₁₆Cer>C_{18:1}Cer>C₂Cer>C₆Cer.

With increasing amounts of C16 Cer Tm elevated with a sharper peak reaching a minimum width around 50% of C₁₆ Cer (FIG. 3A). At higher C₁₆ Cer mole % the peak broadened again.

The same effects were observed when the phase transition of MLV was analyzed by measuring the temperature-dependent changes in turbidity determined as optical density (O.D.) using Carry 300 Bio UV-visible double beam spectrophotometer (as described in Materials and Methods). FIGS. 3C, 3D and 3E show the curves of the 1^{st} derivative of OD. (dO.D./dT) versus temperature of MLV containing C₂ Cer, C₆ Cer and C₁₆ Cer, respectively; these curves resemble the DSC thermograms (compare FIG. 4 and FIG. 5A).

FIG. 3C shows that addition of 12.5 or 25 mole % of C₂ Cer into the HSPC lipid bilayer decreased both the range of phase transition temperature and Tm (the T of maximum charge in dOD/dT) of the HSPC, compared with a sharp peak that was observed for the HSPC alone (FIG. 3C). At 50 and 75 mole % of C₂ Cer the thermograms suggest phase separation. These observations are in agreement with large “free volume” and loose packing of the lipids in the assemblies due to the large ceramide chain mismatch (FIG. 3C).

Similarly and even more striking effect was observed for the MLV comprising C₆ Cer. FIG. 3D shows a sharp single peak for HSPC alone which was broader for lipid dispersions consisting of HSPC containing 12.5 mole % of C₆ Cer, while at 25 and 50 mole % of C₆ Cer in HSPC the lipid dispersions show a split peak due to an additional peak at lower T (FIG. 3D). At 75 mole % C₆ Cer only one broad asymmetric peak having Tm at ˜303° K exists and a shoulder toward the high T. C₁₆ Cer effect is very different from C₂ Cer and C₆ Cer. As shown in FIG. 3E, addition of C₁₆ Cer to HSPC has the opposite effect on Tₘ as the phase transition temperature range and Tₘ of the lipid assemblies was shifted upwards with the addition of increasing mole % of C₁₆ Cer. The shift of the main transition temperature to a higher temperature implied a good mixing and cooperative interaction between HSPC and C₁₆ Cer molecules even at 75 mole % of C₁₆ Cer, although the structure of the aggregate formed may be heterogeneous.

Comparison Between the Thermotropic Behavior of MLV and LUV Consisting of HSPC, ^{2k}PEG-DSPE or ^{2k}PEG-DSG and C₆ Ceramide.

The appearance of two endotherms at the DSC thermograms (FIG. 4) indicates that MLV containing C₆ Cer resulted in either a microscopic (intraliposome) and/or a macroscopic (interparticle) phase separation, which is in agreement with the lower miscibility of HSPC and C₆ Cer than for the HSPC and C₁₆ Cer which may be due to a mismatch in molecular shape of the C₆ Cer compared with that of the phospholipid molecule (FIG. 1B) such mismatch may cause instability.

Effect of ^{2k}PEG-DSPE on Ceramide/HSPC Miscibility

The effect of adding a ^{2K}PEG.-DSPE lipopolymer to the mixture of HSPC and C₆ Cer on the miscibility of ceramide and HSPC was studied using DSC. The liposome composition (MLV and LUV) was determined (as described in Materials and Methods). It was found that in all samples the ratio between ceramides and PL was approximately the same (data not shown).

FIGS. 4A and 4B show the effect of ^{2k}PEG-DSPE on the temperature range and Tₘ of the phase transition of MLV and LUV having HSPC:C₆ Cer mole ratio of 3:1. Results obtained with DSC (FIG. 4) for lipid dispersions (MLV) showed a similar effect to the one observed by measurements of the effect of T on change in O.D. (FIG. 5A).

From FIGS. 5A and 5B it may be concluded that two peaks exist in the thermograms of lipid dispersions containing 0 and 5 mole % of ^{2k}PEG-DSPE, suggesting phase separation (FIG. 5A and FIG. 4). However, after increasing mole % of ^{2K}PEG-DSPE to 7.5 and 10 mole % only one peak remains in the thermogram, suggesting good miscibility of all the components and no phase separation. Addition of ^{2k}PEG-DSPE to 12.5 and 20 mole % the peak at 308° K completely disappeared due to solubilization and formation of micelles. The area under the high temperature peak at 321° K decreased further to almost “no peak”. This may be explained by the formation of mixed micelles at higher mole % of ^{2k}PEG-DSPE, for which ‘cooperativity’ of the phase transition is very low. This is also in agreement with size distribution measurements by DLS. However, the area under the peak at 321° K as measured by DSC increased with the addition of ^{2k}PEG-DSPE, which may support the formation of PEG-DSPE micelles.

Comparing the thermograms of MLV to LUV (FIG. 5A and 5B) show good agreement, indicating that the “MLV” are indeed assemblies containing all lipid components in the same particle. In LUV composed of HSPC:Cer C₆ 3:1 without lipopolymer like in MLV two peaks can be distinguished clearly, although the relative size of the lower temperature peak at 308° K is smaller than in the MLV. Apparently the downsizing (from MLV to LUV) of the liposomes caused a decrease in phase separation, producing LUV which are more homogenous than the MLV. Addition of 10 mole % ^{2k}PEG-DSPE abolished the phase separation completely which indicates a better miscibility between the molecules in the LUV. This indicates that downsizing does not force ceramide out of the membrane but rather improved mixing of lipid components thereby decreasing the level of phase separation, producing a laterally more homogenous bilayer.

The phase transitions of the HSPC LUV containing C₆ Cer and stabilized by neutral ^{2k}PEG-DSG was also studied. The results show that in liposomes consisting of HSPC and 5 or 7.5 mole % of ^{2k}PEG-DSG a sharp peak of phase transition was observed, and that increase of mole % of ^{2k}PEG-DSG increase the Tm (FIG. 5C and 5D). Addition of 11.5 mole % of C₆ Cer preserved the sharp peak and lowered the Tm (FIGS. 5C and 5D). When the 23 mole % of C₆ Cer was added to the HSPC lipid bilayer having 5 mole % of ^{2k}PEG-DSG the peak describing the phase transition became broader which agrees with non-ideal mixing of the liposome components (FIG. 5C). However, addition of 7.5 mole % of ^{2k}PEG-DSG restored the peak width, which suggests an improved miscibility of the liposome components in agreement with the results presented in FIG. 5B for the effect of ^{2K}PEG-DSPE.

Thus, the addition of a lipopolymer such as ^{2k}PEG-DSPE or ^{2k}PEG-DSG to liposomes containing C₆ Cer ceramides improved the miscibility of the lipid components in MLV and LUV, therefore reducing lateral phase separation (lateral phase separation introduce instability due to defects in the bilayer packing) and therefore increasing liposomes instability coexistence of SO and LD phases.

Effect of LUV compositions of Tire Specific Compressibility of the Liposome Membrane

Large unilamellar liposomes (LUV≦100 nm) composed of various ceramides (different chain lengths: short (C₂ Cer), medium (C₆ Cer), and long (C₁₆ Cer), liposome-forming PL (EPC or HSPC), and ^{2k}PEG-DSPE were characterized for their compressibility.

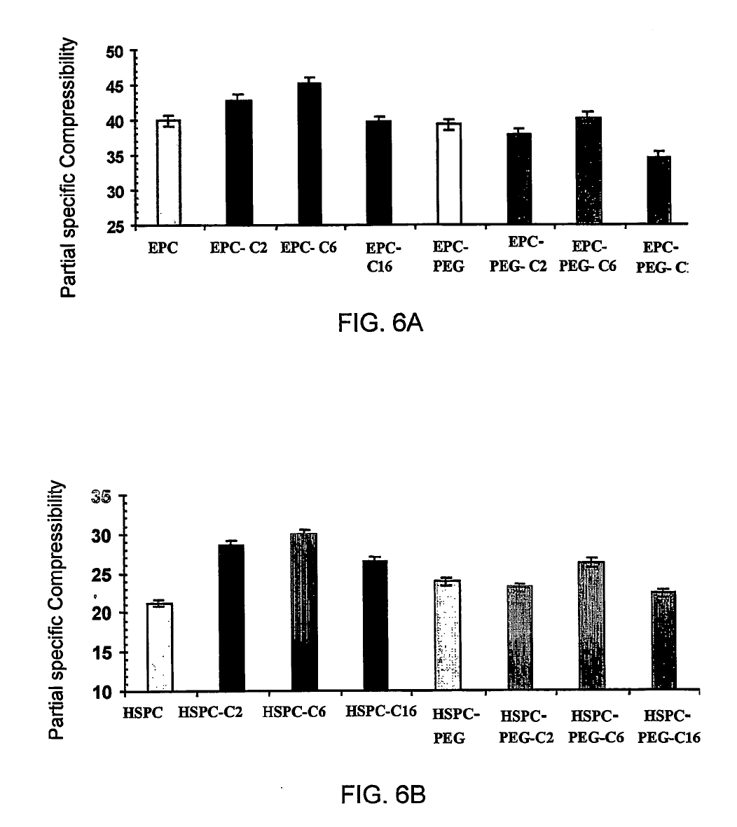

The lipid compressibility was calculated from the acoustical and volumetric measurements (as described in the Materials and Methods). FIGS. 6A and 6B show the influence of PL acyl chain saturation and the presence of ^{2k}PEG-DSPE (7.5 mole %) on the lipid bilayer compressibility of LUW having different ceramides (C₂ Cer, C₆ Cer and C₁₆ Cer).

In particular, the results show that LUV consisting of EPC (i.e. unsaturated fluid phospholipid) had a higher compressibility as compared to LUV composed of HSPC (saturated solid phospholipid) which is consistent with the physical state of the membrane (LD for EPC and so for HSPC lipid bilayers).

FIGS. 6A and 6B also present the relative changes in compressibility of the liposomes as a result of adding the lipopolymer ^{2k}PEG-DSPE. As can be seen, addition of 7.5 mole % of ^{2k}PEG-DSPE decreased the compressibility of LUV consisting of EPC or HSPC, indicating that liposomes comprising a lipopolymer in their bilayers, such as ^{2k}PEG-DSPE, are more tightly packed. Tight packing of liposomes also agrees with increased stability of the liposome formulations.

The results show that LUV consisting of EPC or HSPC with or without the lipopolymer, ^{2k}PEG-DSPE, and comprising C₆ Cer in their lipid bilayers possess a high compressibility (FIGS. 6A and 6B) as compared to other respective liposomal ceramides, namely, such liposomes are less tightly packed than similar liposomes with other ceramides. This is in agreement with the results from the measurement of thermotropic behavior of liposomes and stability of LUV (hereinbefore).

Comparing between the various ceramides, LUV consisting of EPC or HSPC with or without ^{2k}PEG-DSPE and having C₁₆ Cer in their lipid bilayers showed the lowest compressibility value, which indicates the smaller free volume. Also, these results are consistent with the results of the thermotropic behavior of the liposomes that show good mixing of the PL and C₁₆ Cer and cooperative interaction between HSPC and-C₁₆ Cer.

Assembly Stability

Large unilamellar liposomes (LUV) composed of various ceramides, bilayer-forming PLs (either EPC or HSPC), and lipopolymers, such as ^{2k}PEG-DSPE and ^{2k}PEG-DSG were analyzed for their physical and chemical stability upon storage at 4° C. in citrate buffer (pH 7.0).

Chemical Stability

The main parameter for chemical stability studied is stability of acyl ester bond. This was done by following directly the release of non-esterified fatty acids (NEFA) which are released as a result of PL hydrolysis and indirectly through pH measurements. The results indicate that when stored at 4° C. in citrate buffer, pH 7.0 all liposome formulations were chemically stable for at least 6 months as the level of NEFA did not increase above 3%. Similarly, no change from the initial liposome dispersion pH was found in all LUV preparation during storage under these conditions.

Physical Stability Upon Storage at 4° C.

Physical instability of the assemblies includes aggregation and/or fusion of liposomes (measured as changes in particle size distribution by DLS) and macroscopic de-mixing of the components leading to ceramide being sequestered out of the liposome to form a ceramide-rich precipitate (measured by TLC after centrifugation in which pellet and supernatant were separated (see Materials and Methods).

Table 4 above shows the physical stability of LUV during storage at 4° C. based on changes in ceramide/PL ratio (phospholipid content was determined as organic phosphorus by the Bartlett method and ceramide content was measure by TLC as described in Materials and Methods).

In general, liposomes based on EPC were more physically stable than liposomes based on HSPC, although both show long-term stability.

Further, liposomes containing ceramide having short (C₂ Cer and C₄ Cer) and especially medium (C₆ Cer) acyl chain were slightly less stable than liposomes composed from medium (C₈ Cer) or long (C₁₆ Cer) chain ceramides. These results are consistent with the thermotropic behavior of unsized lipid dispersions and of LUV (FIGS. 3, 4 and 5A-5D), showing that at 25 or 50 mole % of C₆ Cer in HSPC there is a phase separation. LUV containing long chain (C₁₆ Cer) ceramide were the most stable, which is consistent with the thermotropic behavior of liposomes composed of C₁₆ Cer and HSPC which showed a better miscibility of C₁₆ Cer with HSPC than with the other ceramides used.

LUV containing ^{2k}PEG-DSPE in the lipid bilayer were more physically stable than liposomes lacking ^{2k}PEG-DSPE (as shown, for examples in formulation No. 18,19, 20, and 19,20, in Table 4). This is in agreement with the data showing that the addition of ^{2k}PEG-DSPE into HSPC lipid bilayer modify the thermotropic behavior of LUV and that such liposomes have an improved packing and stability in the presence of ^{2k}PEG-DSPE (FIGS. 4, 5A, and 5B).

In addition, it was found that when cholesterol is included in the liposome to form a formulation of EPC/Chol/^{2k}PEG-DSPE/C₆ Cer (44/37/7.5/23) the resulting liposomes were physically unstable and decomposed within a week (No. 26 in Table 4 above).

When comparing the liposomes with the same mole % of ceramides the relative stability during storage at 4° C. was as follows:

EPC/HSPC:^{2k}PEG-DSPE:C₁₆Cer/C₈Cer=EPC:^{2k}PEG-DSPE:C₂Cer>EPC:^{2k}PEGDSPE: C₄Cer>EPC:^{2k}PEGDSPE:C₆Cer>HSPC:^{2k}PEG-DSPE:C₂Cer>HSPC:^{2k}PEGDSPE:C₄Cer>EPC:C₆Cer>HSPC:^{2k}PEG-DSPE:C₆Cer>HSPC:C₆Cer>EPC:^{2k}PEG-DSG:C₆Cer=HSPC:^{2k}PEG-DSG:C₆Cer

Measurement of Assembly Size Distribution in Serum

As the lipid assemblies containing ceramides (i.e., micelles and liposomes) are aimed for intravenous (i.v.) administration it was important to study and evaluate the effect of serum on the physical stability of the liposomes containing ceramides. Therefore, the changes in size of different liposomal formulations comprising short (C₂Cer), medium (C₆Cer) and long (C₁₆Cer) ceramides before and after exposure to serum (FCS) was measured by DLS (as described in Materials and Methods). Two independent methods were used: DLS (Table 6) and turbidity measurements (Table 7). It was found that the size of the liposomes did not change significantly with formulations lacking ceramides or those which include C₂ and C₆Cer when brought into contact with sera.

However. LUV which consisted of EPC/PEG-DSPE and ceramide C₁₆ increased in size, probably due to aggregation (Table 6). These measurements were consistent with measurements of turbidity (Table 7).

TABLE 6
Effect of serum addition on size of various LUV

| | Liposome formulations | Initial size | Size of LUV in |
|---|---|---|---|
| No. | (mole ratio) | (nm) | serum (1:1) (nm) |
| 1 | EPC:^{2k}PEG-DSPE (69.5:7.5) | 98 | 132 |
| 2 | EPC:^{2k}PEG-DSPE:C₂Cer | 104 | 112 |
| | (69.5:7.5:23) | | |
| 3 | EPC:^{2k}PEG-DSPE:C₆Cer | 108 | 138 |
| | (69.5:7.5:23) | | |
| 4 | EPC:^{2k}PEG-DSPE:C₁₆Cer | 140 | 894 |
| | (69.5:7.5:23) | | |

Turbidity Measurement of Turbidity of the Different LUV Formulations in the Serum

In addition to direct size distribution analysis by DLS, changes in size were also followed through changes in the ratio of turbidity (determined by OD) at 300 and 600 nm (for more details see Materials and Methods, and Barenholz and Amselem, 1993). This approach is complementary as it relates to changes in the dispersion homogeneity with respect to particle size. The results (Table 7) confirm those presented in Table 6, which show that only liposome formulations consisting of EPC, ^{2k}PEG-DSPE and long-chain C₁₆ Cer aggregate in the presence of serum (Table 7). Based on the change, the TR=OD₁/OD₂ for EPC/PEG-DSPE/C₁₆ Cer decreased dramatically for LUV in the presence of serum from 7.59 to 4.2 respectively compared with almost no change for LUV lacking ceramides or those containing either C₂ or C₆Cer indicating that serum induced aggregation with the long chain C₁₆ Cer. This finding thus suggests that liposomes containing C₂ and C₆ Cer remain stable in serum.

TABLE 7
Effect of Serum of 300 nm/600 nm Turbidity ratio (TR) of LUV

| | Liposome formulations | | TR of LUV in |
|---|---|---|---|
| No. | (mole ratio) | TR of LUV | 50% serum |
| 1 | EPC:^{2k}PEG-DSPE (69.5:7.5) | 7.96 | 8.6 |
| 2 | EPC:^{2k}PEG-DSPE:C₂Cer | 8.38 | 7.9 |
| | (69.5:7.5:23) | | |
| 3 | EPC:^{2k}PEG-DSPE:C₆Cer | 7.89 | 7.7 |
| | (69.5:7.5:23) | | |
| 4 | EPC:^{2k}PEG:DSPE:C₁₆Cer | 7.59 | 4.2 |
| | (69.5:7.5:23) | | |

Cytotoxic Activity

So far most studies on the biological activity of ceramides were focused mainly in cells in culture. The ceramides were introduced to cell medium either in ethanol or in ethanol:dodecane (98:2 by volume) dispersion [Hirabayashi et al., FEBS Letters, 358:211-214, (1995)]. The working hypothesis of using such dispersions is that the ethanol or ethanol:dodecane are a means to disperse the ceramides in the aqueous tissue culture medium, thereby making it available to serum proteins (mainly albumin), which will deliver the ceramides to the cells in culture [Hannun et al., Methods in Enzymology, 88:444-448, (2000)].

In this study the above two methods of dispersion via ethanol or ethanol:dodecane were compared with the use of liposomes or micelles containing ceramides as a means to introduce the ceramides to cells in culture. Cytotoxicity was used as an endpoint for ceramide biological activity. In addition, the ability of all these three methods to be used for in vivo delivery of ceramides was assessed firstly by comparing the effect of serum on the size distribution of the dispersion particles, and by evaluating their feasibility to be injected to mice.

The influence of the method of introducing ceramide to the cells in culture on cytotoxic activity of the different ceramides, such as C₂Cer, C₆Cer and C₁₆Cer was evaluated. To this end C₂Cer, C₆Cer and C₁₆Cer were dispersed in ethanol or in the ethanol:dodecane system and the cytotoxic activity of these dispersions determined as IC₅₀ was examined by MB assay against C-26 cells (Methods). It was found that C₂Cer or C₆Cer in the ethanol:dodecane system were less cytotoxic then ceramides dissolved in ethanol alone (Table 8A). However, C₁₆Cer dissolved in ethanol:dodecane was more cytotoxic then C₁₆Cer dissolved in ethanol only (Table 8A).

TABLE 8A
Cytotoxic activity

| | IC₅₀ (μM), | IC₅₀ (μM), | IC₅₀ (μM), |
|---|---|---|---|
| Type of ceramide | 4 hr | 24 hr | 72 hr |
| C₂Cer (in ethanol) | >60.0 | >30.0 | 20.0 ± 1 |
| C₂Cer (in ethanol:dodecane) | >60.0 | >50.0 | 43.0 |
| C₆Cer (in ethanol) | 11.0 ± 5 | 4.1 ± 1.6 | 2.9 ± 1.2 |
| C₆Cer (in ethanol:dodecane) | >30.0 | >30 | 44.0 |
| C₁₆Cer (in ethanol) | >100.0 | >100.0 | 80.0 |
| C₁₆Cer (in ethanol:dodecane) | 63.0 | 50.0 | 46.0 |

All ceramides and all liposomal formulations with different ceramides (C₂ Cer, C₄ Cer, C₆ Cer, C₈ Cer and C₁₆ Cer) were tested for their cytotoxic activity on two tumor cell lines. All ceramides, either by themselves as ceramide solution in ethanol or as part of a liposomal formulation, were cytotoxic, although to a different extent (depending mainly on the type of ceramide). The IC₅₀ values of liposomal ceramides resemble those of the free ceramide (Table 9B).

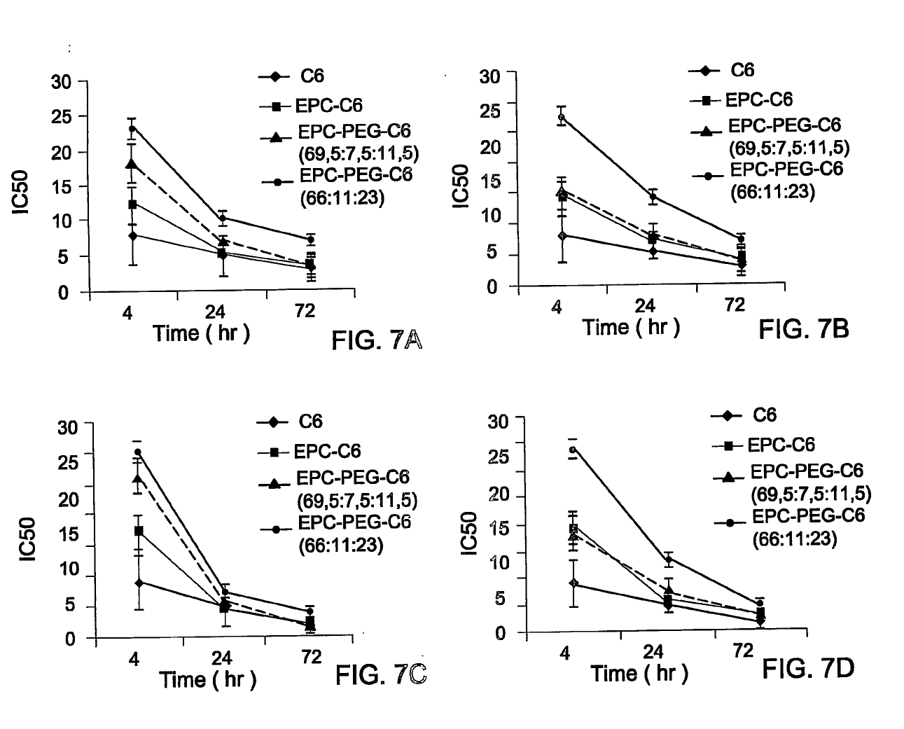

In general liposomal ceramides were slightly less active than free ceramides especially at the short incubation time (4 h), while at 72 h incubation activity of liposomal ceramide was identical to ceramide in ethanol. For liposomal ceramides the presence of lipopolymers such as ^{2K}PEG-DSPE or ^{2K}PEG-DSG lowered somewhat the ceramide efficacy mainly at the short incubation times in a mole %-dependent manner. The higher the lipopolymer mole % in the liposome the lower is the ceramide activity (Table 8B and FIG. 7). Albeit the above, the importance of effectively loading ceramides and slowing down their release of the lipid assemblies onto lipid assemblies, such as liposomes, for the delivery of such biologically active substances, should be well appreciated to those versed in the art, even at the price of slightly reducing its efficacy in tissue culture.

It was also found that LUV comprising C₆Cer and ^{2k}PEG-DSG were also cytotoxic, however to a lower extend as compared to ceramide liposomes stabilized by ^{2k}PEG-DSPE and that the cytotoxic effect of ceramide of ^{2k}PEG-DSG type liposome was expressed slower than for liposomes lacking PEG-DSPE (Table 8B).

TABLE 8B
Cytotoxic activity

| | IC₅₀ (μM), 4 hr | | IC₅₀ (μM), 24 hr | | IC₅₀ (μM), 72 hr | |
|---|---|---|---|---|---|---|
| Formulation (mole % ratio) | OV-1063 | C-26 | OV-1063 | C-26 | OV-1063 | C-26 |
| C₂Cer (in ethanol) | >80.0 | >60.0 | >30.0 | >30 | 25.0 ± 1.9 | 20.0 ± 1 |
| EPC:^{2k}PEG-DSPE:C₂Cer | >80.0 | >60.0 | 53.0 ± 10 | 54.0 ± 9 | 24.0 ± 7 | 19.0 ± 5 |
| (69.5:7.5:23) | | | | | | |
| HSPC:^{2k}PEG-DSPE:C₂Cer | >80.0 | >60.0 | 70.0 | >60.0 | 25.0 ± 3 | 32.0 ± 3 |
| (69.5:7.5:23) | | | | | | |
| C₄Cer (in ethanol) | 10.0 ± 2.7 | 15.5 ± 3.5 | 5.9 ± 0.14 | 6.25 ± 0.35 | 3.95 ± 0.6 | 3.75 ± 0.35 |
| EPC:^{2k}PEG-DSPE:C₄Cer* | 14.0 ± 4 | 18.0 ± 0 | 6.5 ± 1.9 | 5.5 ± 2 | 1.5 ± 0.58 | 3.25 ± 1.1 |
| (69.5:7.5:23) | | | | | | |
| HSPC:^{2k}PEG-DSPE:C₄Cer | 25.0 ± 3 | 17.0 ± 0.9 | 13.0 ± 4 | 8.0 ± 2 | 3.5 ± 0.5 | 4.0 ± 2 |
| (69.5:7.5:23) | | | | | | |
| C₆Cer (in ethanol) | 8.0 ± 0.1 | 11.0 ± 5 | 4.5 ± 0.7 | 4.1 ± 1.6 | 2.3 ± 1 | 2.9 ± 1.2 |
| EPC:^{2k}PEG-DSPE:C₆Cer* | 14.0 ± 5 | 21.0 ± 6 | 6.7 ± 3 | 4.75 ± 1.4 | 2.5 ± 0.7 | 2.0 ± 0.5 |
| (81:7.5:11.5) | | | | | | |
| EPC:^{2k}PEG-DSG:C₆Cer | Not done | 60.0 | Not done | 24.0 | Not done | 8.5 |
| (81:7.5:11.5) | | | | | | |
| HSPC:^{2k}PEG-DSPE:C₆Cer | 15.0 ± 3 | 18.0 ± 2.8 | 7.5 ± 0.7 | 7.0 ± 2.8 | 4.2 ± 1.7 | 3.2 ± 1.7 |
| (81:7.5:11.5) | | | | | | |
| HSPC:^{2k}PEG-DSG:C₆Cer | Not done | 62.0 | Not done | 14.0 | Not done | 5.8 |
| (81:7.5:11.5) | | | | | | |
| C₈Cer (in ethanol) | >40.0 | >40.0 | 23.7 ± 1 | 23.0 ± 1.4 | 19.0 ± 1.4 | 21.5 ± 2.1 |
| EPC:^{2k}PEG-DSPE:C₈Cer | >40.0 | >40.0 | 27.0 ± 4 | 23.0 ± 3.5 | 12.5 ± 2.5 | 16.2 ± 2.8 |
| (69.5:7.5:23) | | | | | | |
| HSPC:^{2k}PEG-DSPE:C₈Cer | >40.0 | >40.0 | 31.0 ± 0.8 | 43.0 ± 3.1 | 12.0 ± 3.9 | 16.9 ± 3.4 |
| (69.5:7.5:23) | | | | | | |
| C₁₆Cer (in ethanol) | >100.0 | >100.0 | >100.0 | >100.0 | 90.0 | 80.0 |
| EPC:^{2k}PEG-DSPE:C₁₆Cer | >100.0 | >100.0 | >100.0 | >100.0 | 84.0 | 79.0 |
| (69.5:7.5:23) | | | | | | |
| HSPC:^{2k}PEG-DSPE:C₁₆Cer | >100.0 | >100.0 | >100.0 | >100.0 | 98.0 | 100.0 |
| (69.5:7.5:23) | | | | | | |

*most cytotoxic assemblies

Cell Uptake Studies
Uptake and Metabolism of Free or Uncharged Liposomal C₆ C into Cells in Culture

The uptake and metabolism of radiolabelled liposomal or free C₆Cer were studied in C-26 colon carcinoma cells. Cells were incubated with either free ¹⁴C₆Cer (in ethanol) or with liposomal C₆Cer. Total lipids were extracted and the level of uptake and metabolites were determined from cells and growth medium as described in Materials and Methods.

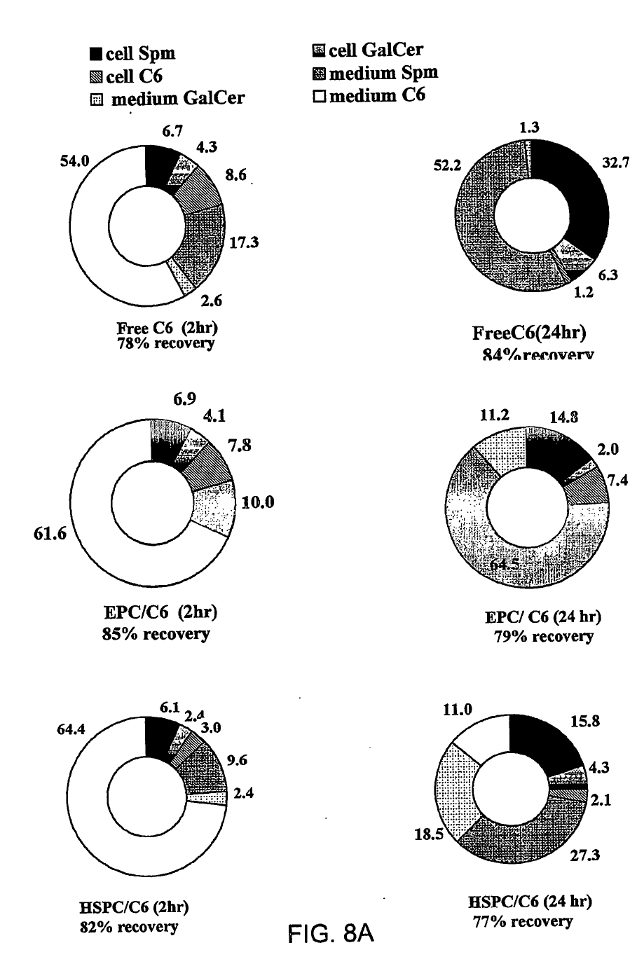

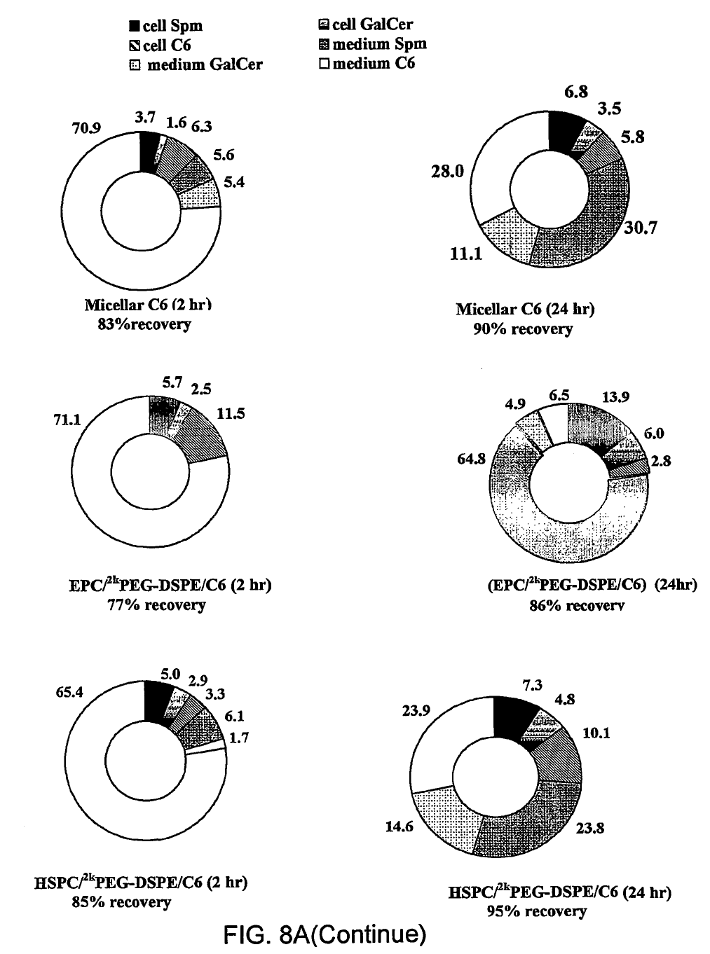

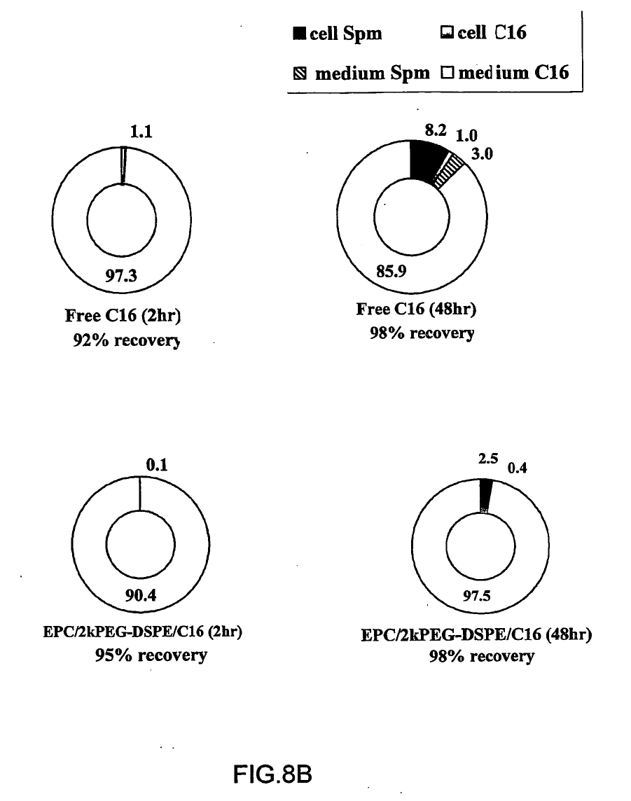

The results presented in FIG. 8A and Table 9 demonstrate that free or liposomal C₆Cer were efficiently and similarly taken and metabolized by C-26 cells in time-dependent manner. C₆ splingomyelin (C₆SPM) present in cell medium and in the cells was the main metabolite, and C₆ galactocerebroside (C₆GalCer), also present in cell growth medium and in the cells, was the second (minor metabolite. The residual (unmetabolized) C₆Cer in cell growth medium and the C₆Cer level in the cells were also determined. The sum of these six fractions enables to calculate at all time points studied the percent recovery of C₆ ceramide added to the cells growth medium (as ethanol dispersion or as part of the liposome) at time zero.

TABLE 9
C-26 cells uptake of ceramides assemblies

| | | | | Cell | Lipid/ | Cell | Cell | Lipid/ | Cell | Cell | Lipid/ |
|---|---|---|---|---|---|---|---|---|---|---|---|
| | Initial | | Cell uptake | uptake | Cer | uptake | uptake | Cer | uptake | uptake | Cer |
| | lipid/ | Molecule | (nmole) | (%) | ratio | (nmole) | (%) | ratio | (nmole) | (%) | ratio |
| Lipid assembles | Cer ratio | followed | 2 hr | | | 24 hr | | | 72 hr | | |
| C₆Cer | | ¹⁴C₆Cer | 13.7 | 39.9 | | 34.5 | 93.7 | | 24.5 | 72 | |
| ^{2k}PEG-DSPE/C₆Cer* | 1.86 | ¹⁴C₆Cer | 7.3 | 21.2 | | 19.8 | 57.8 | | not | not | not |
| | | | | | | | | | done | done | done |
| EPC/^{2k}PEG-DSPE/ | 6.7 | ¹⁴C₆Cer | 6.3 | 19.7 | 0.9 | 28.2 | 87.6 | 0.8 | 31.4 | 97.4 | 0.1 |
| C₆Cer^{Δ} | | ³H-DPPC | 5.9 | 2.7 | | 21.5 | 9.9 | | 3.3 | 1.5 | |
| EPC/C₆Cer^{Δ} | 6.7 | ¹⁴C₆Cer | 7.4 | 28.8 | 0.9 | 22.7 | 88.7 | 0.9 | 20.0 | 78.4 | 1.1 |
| | | ³H-DPPC | 6.4 | 3.3 | | 20.2 | 10.4 | | 22.5 | 11.6 | |
| HSPC/^{2k}PEG-DSPE/ | 6.7 | ¹⁴C₆Cer | 4.9 | 19 | 0.7 | 15.6 | 60.7 | 0.6 | not | not | not |
| C₆Cer^{Δ} | | ³H-DPPC | 5.24 | 2.7 | | 9.9 | 5.1 | | done | done | done |
| HSPC/C₆Cer^{Δ} | 6.7 | ¹⁴C₆Cer | 6 | 23.6 | 0.9 | 17.4 | 67.9 | 0.8 | not | not | not |
| | | ³H-DPPC | 5.6 | 2.9 | | 13.8 | 7.1 | | done | done | done |
| DOTAP/DOPE/EPC/ | 6.7 | ¹⁴C₆Cer | 6.7 | 19.0 | 24.3 | not | not | not | not | not | not |
| C₆Cer^{Δ} | | ³H-DPPC | 25.7 | 70 | | done | done | done | done | done | done |

*micelles;
^{Δ}liposomes

Similar uptake and metabolism follow-up studies were performed on formulations which include ¹⁴C-C₁₆Cer.

FIG. 8B shows that C₁₆Cer is taken up by the cells at a much lower rate than C₆Cer, although C₁₆Cer is also metabolized into C₁₆ SPM at a much slower rate.

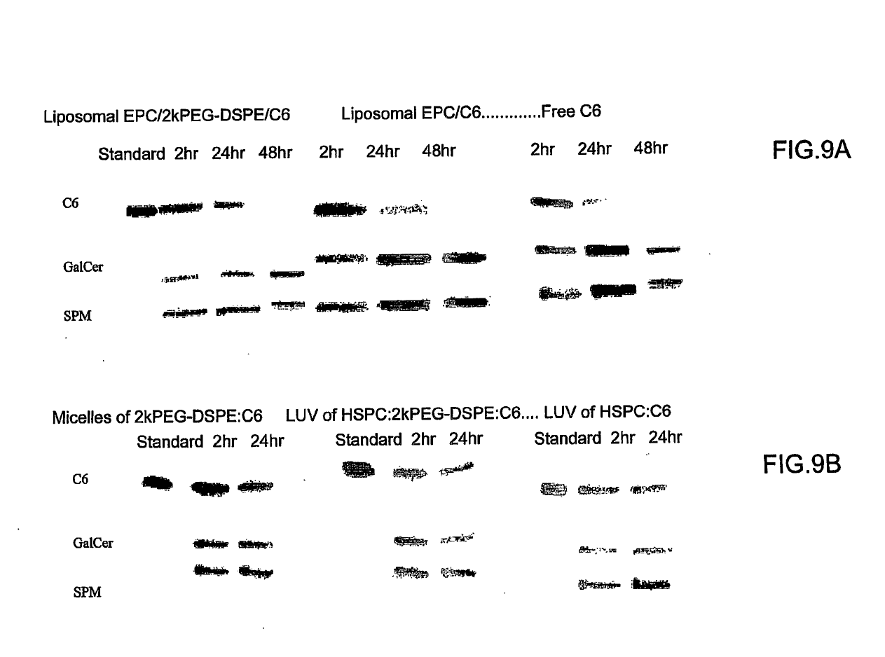

FIG. 9A and FIG. 9B show radioactivity (C) chromatograms of silica gel TLC of cells+ medium lipid extracts processed and analyzed as described in Materials and Methods. The results are in good agreement with those described in Table 9. The results presented in FIG. 9A and 9B show that C₆Cer was taken by the cells either from the free, liposomal or micellar form, however, to a different extent. FIG. 9A and B demonstrated that after 2 hr of incubation part of the C₆Cer taken by the cells remain at the form of C₆Cer and the rest was metabolized mostly into the SPM or GlcCer. After 24 hr or 48 hr of incubation most of the C₆Cer was metabolized into the SPM or GlcCer metabolites.

The results of cell uptake and metabolism of ¹⁴C-C₁₆ ceramide which are a component of different lipid assemblies, as presented in FIGS. 8A to 9B and in Table 9 can be summarized as follows:
(1) The recovery of total ¹⁴C (Cer) and ³H (DPPC) radioactivity was higher than 75% and in most samples higher than 80%, giving the data good reliability and accountability.
(2) To a large extent differences regarding ceramide uptake between the various lipid assemblies were much more pronounced at shorter incubation time (2 h). These differences disappeared after longer incubation periods (24 or 72 h) for EPC-based liposomes, and remain similar for ^{2K}PEG-DSPE:C₆Cer micelles and for liposomes based on HSPC (with and without PEG-DSPE).
(3) After 2 h incubation uptake of C₆Cer is in the following order:
   Free C₆Cer>EPC:C₆Cer Lip>HSPC:C₆Cer Lip>PEG-DSPE/C6Cer micelles>EPC:^{2K}PEG-DSPE:C₆Cer>HSPC:PEG-DSPE:C₆Cer Lip.
(4) After 24 h the smallest cellular uptake was observed for ^{2K}PEG-DSPE:C₆Cer micelles and HSPC:^{2k}PEG-PEG:C₆ Cer liposomes. Namely, regarding the liposome-forming lipid uptake of ¹⁴C₆Cer is slower from HSPC than of EPC liposomes and for both PCs the presence of ^{2K}PEG-DSPE slows down ceramide uptake by the cells.
(5) Uptake of ³H DPPC, which served as a marker for the liposome PCs (used as liposome forming lipid, also referred to as lipid matrix), was much lower than for the C₆ ceramide (˜ 1/10- 1/7, e.g. see ratio of Lipid/Cer . . . Cer/PC . . . in cells (1.1-0.6) with the ratio in the liposomes of 6.7 (Table 9)).
(6) Metabolism of C₆Cer by the cells in culture reflected cell uptake, namely the higher the uptake the larger the fraction of the metabolites. Only two metabolites were observed using silica TLC as described in FIGS. 8 and 9, and in Materials and Methods. C₆ sphingomyelin (SPM) and C₆ galactocerebroside (Gal Cer). In all cases the C₆ SPM was the main metabolite and the first to appear. Most of the C₆SPM was found in the cell growth medium. After 24 h all C₆Cer of EPC:C₆Cer liposomes was taken up by the C₂₆ cells and mostly metabolized (64.5%) to C₆ SPM found in cell medium, 14.8% as C₆ SPM in cells and only 7.4% remain as C₆Cer in the cells. No C₆Cer remained in cell medium. The presence of ^{2K}PEG-DSPE in the liposomes slowed down both uptake and metabolism of C₆Cer. The above results may suggest that C₆Cer is taken up by cells by itself without the PC either after being released from the lipid assemblies, or by diffusion from the lipid assemblies during their collision with the cells. The PC of the assemblies is taken up by the cells at a much lower rate, either through exchange and/or transfer between liposomes and cells or by the small uptake of intact liposomes by the cells. Both mechanisms have been shown in other cell cultures in the past for PC [Yechiel E. and Barenholz Y. J Biol Chem. Aug. 5, 1985;260(16):9123-31.]

Once taken up by the cells C₆Cer is metabolized in the C₂₆ cells by well-established pathways mainly to C₆ SPM and to a lesser extent to C₆ Gal Cer.

To examine whether metabolism occurs due to enzymes released by the cells into the medium, free C₆Cer, EPC/^{2k}PEG-DSPE/C₆Cer or EPC/C₆Cer were incubated with two types of media: (a) cell derived medium (taken from C₂₆ cells)or (b) fresh medium. It was found that there was no metabolitic activity in the fresh medium, while 9% of free C₆Cer, 4.5% of ceramide derived from EPC/^{2k}PEG-DSPE/C₆ and 6% of ceramide derived from EPC/C₆ Cer were converted into C₆ SPM in the cell derived medium (data not shown).

Cell Uptake of C₆Cer from Positively Charged Lipid Assemblies

In order to determine the role of lipid assemblies' electrical charge in cell uptake of the biological active and in the specific example of C₆Cer in lipid assemblies composed from a mixture of a cationic lipid, DOTAP, neutral lipid, DOPE, and C₆Cer containing radioactively labeled ¹⁴C C₆Cer and ³H-DPPC as tracers, labeled ¹⁴C C₆Cer and [³H] DPPC were prepared. The uptake of the ¹⁴C C₆Cer and ³H-DPPC after 2 hr of incubation with C-26 cells was determined as described above. The results show that the fact that the liposomes were positively charged did not accelerate the rate of ceramide uptake by the cells (19%) and relatively to liposomes or micelles lacking positive charge (Table 9), although the rate of ³H-DPPC uptake (70%) was highly accelerated (at least 20 fold compared with noncationic liposomes (compare Table 10 and 9).

In addition, the ratio between ³H-DPPC and ¹⁴C-C₆Cer was much higher inside the cells (24.3) than that in the originally formed assemblies (6.7). This may also suggest the uptake of ceramides by the cells was independent (and faster) from the uptake of the lipid assembly.

Assessment of Apoptosis

In most cell types, phosphatidylserine (PS) a lipid normally confined to the inner leaflet of the plasma membrane, is exported to the outer plasma membrane leaflet in the early stage of apoptosis. PS exposure in treated C-26 and OV-1063 cells was detected by staining with MC 540, which has a strong affinity to PS. Chromatin morphology was assessed by staining with DAPI, which preferentially stains dsDNA.

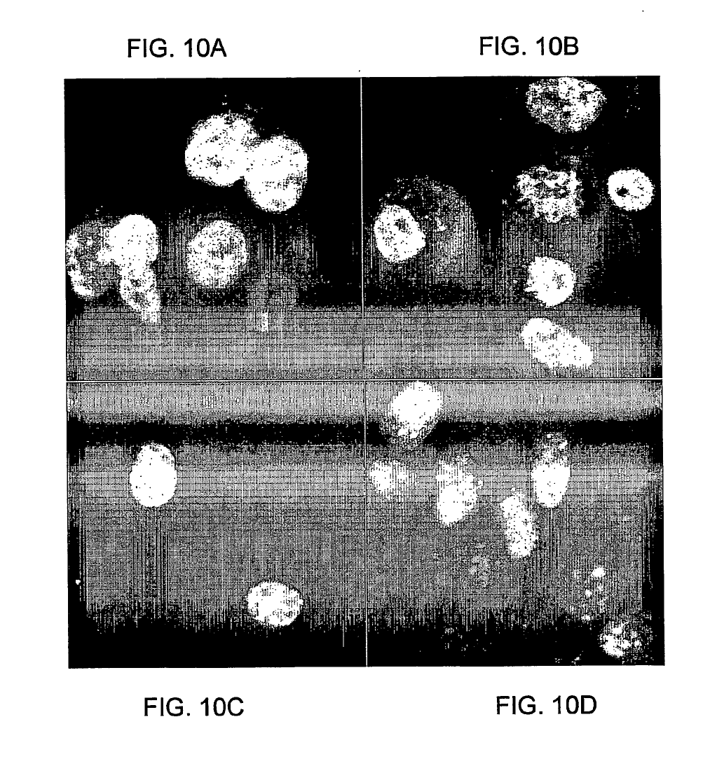

FIG. 10A-10D show distinct features of apoptosis in OV-1063 cells treated with liposomal C₆Cer. This is evidenced by the appearance of red fluorescence in the cell membrane (marked in FIG. 10B by the triangles). The results of this staining show that a large proportion of the OV-1063 cells appeared to be apoptotic after 4 h of treatment with 15 μM C₆ Cer delivered as EPC:^{2k}PEG-DSPE:C₆Cer (81:7.5:11.5) liposomes (FIG. 10B, as compared to non-treated cells shown in FIG. 10A). However, no such fluorescence signal was found in C-26 cells treated similarly (FIG. 10D, compared to non-treated cells shown in FIG. 10C). This suggests a non-apoptotic mechanism of action of C₆Cer in C₂₆ cell culture.

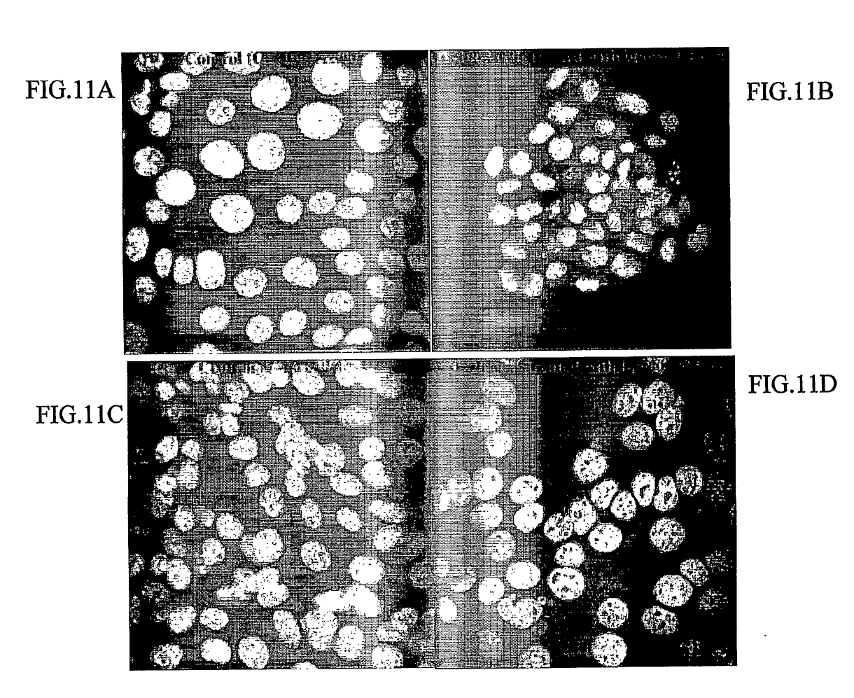

The difference in C₆Cer induced cell death in OV-1063 and C₂₆ cells was also confirmed when morphological signs of apoptosis were followed including nucleoplasm and cytoplasm condensation with a pronounced decrease in cell volume, chromatin condensation, plasma membrane blebbing, and degeneration of the nucleus into membrane-bound apoptotic bodies. While all these apoptotic signs were highly pronounced in OV-1063 tumor cells treated with different liposomal ceramide formulations (determined by staining of dsDNA with Hoechst-33342, which was measured with the aid of CLSM), they were lacking, or much less pronounced, in C₂₆ cells. Based on these criteria the results presented in FIG. 11 show that a large proportion of OV-1063 cells but not of C-26 cells treated for 4 hr with 15 μM of EPC: ^{2k}PEG-DSPE:C₆Cer (81:7.5:11.5) C₆Cer liposomes became apoptotic. Table 10 shows that a large proportion of OV 1063 cells, but not of C-26 cells treated for 16 and 24 hr with the different ceramides (C₂Cer, C₄Cer, C₆Cer, and C₁₆Cer) and different lipid assemblies containing these ceramides become apoptotic.

TABLE 10
Percent of apoptotic cells calculated from confocal microscopy
images (staining of dsDNA with Hoechst-33342)

| | Apoptotic OV-1063 | | | |
|---|---|---|---|---|
| | cells (% of total | | Apoptotic C-26 cells (% | |
| | cell number | | of total cell number) | |
| Treatment | 16 hr | 24 hr | 16 hr | 24 hr |
| Control | 4 | 3 | 2 | 4 |
| Free C₂Cer | 48 | 60 | 7 | 14 |
| EPC:PEG-DSPE:C₂Cer | 48 | 73 | 28 | 41 |
| (69.5:7.5:23) | | | | |
| C₄Cer | not done | 58 | not done | 10 |
| EPC:PEG-DSPE:C₄Cer | not done | 55 | not done | 16 |
| (69.5:7.5:23) | | | | |
| Free C₆Cer | 53 | 61 | 9 | 18 |
| EPC:PEG-DSPE:C₆Cer | 51 | 59 | 8 | 19 |
| (69.5:7.5:23) | | | | |
| Free C₁₆Cer | note done | 17 | not done | 9 |
| EPC:PEG-DSPE:C₁₆Cer | note done | 18 | not done | 16 |
| (69.5:7.5:23) | | | | |

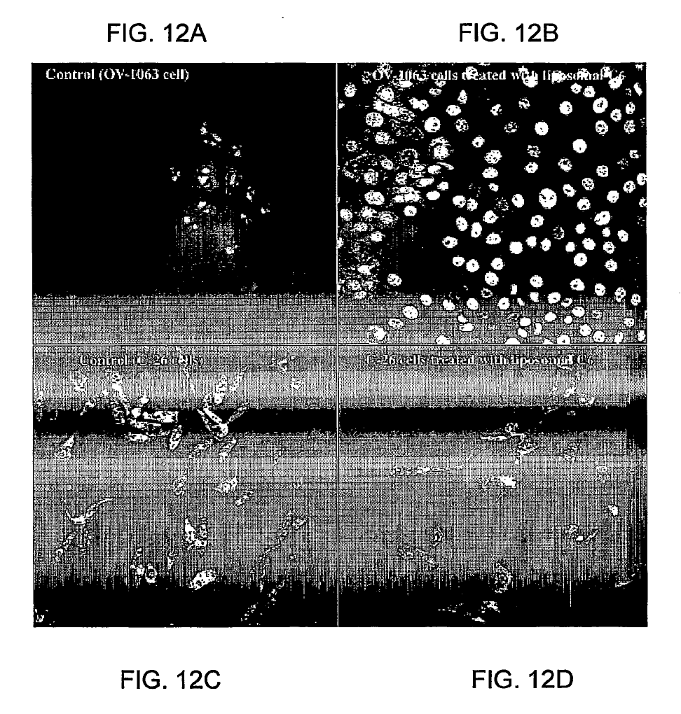

Moreover, the TUNEL method (measuring fragmentation of DNA) showed that a large proportion of OV-1063 cells, but not of C-26 cells treated with IC₅₀ values of C₆Cer delivered as EPC:^{2k}PEG-DSPE:C₆Cer (81:7.5:11.5) became apoptotic after 24 hr of treatment (FIG. 12).

Biochemically, members of the caspase (CED-3/ICE) family of proteases have been found to be crucial mediators of the complex events associated with apoptosis [Thornberry, N. A. and Lazebnic, Y. Science 281:1312-1316 (1998)]. In particular, the activation of caspase-3, which cleaves a number of different proteins, including poly (ADP-ribose) polymerase (PARP), protein kinase C and actin, has been shown to be important for the initiation of apoptosis [Villa, P. et al. Trends Biochem. Sci., 22:388-393 (1997)].

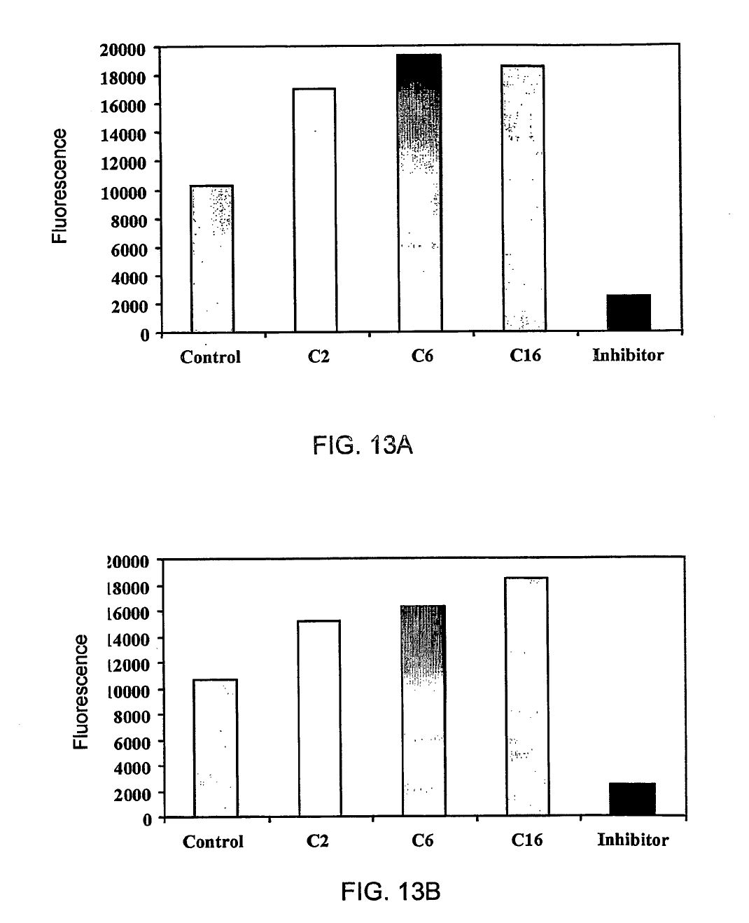

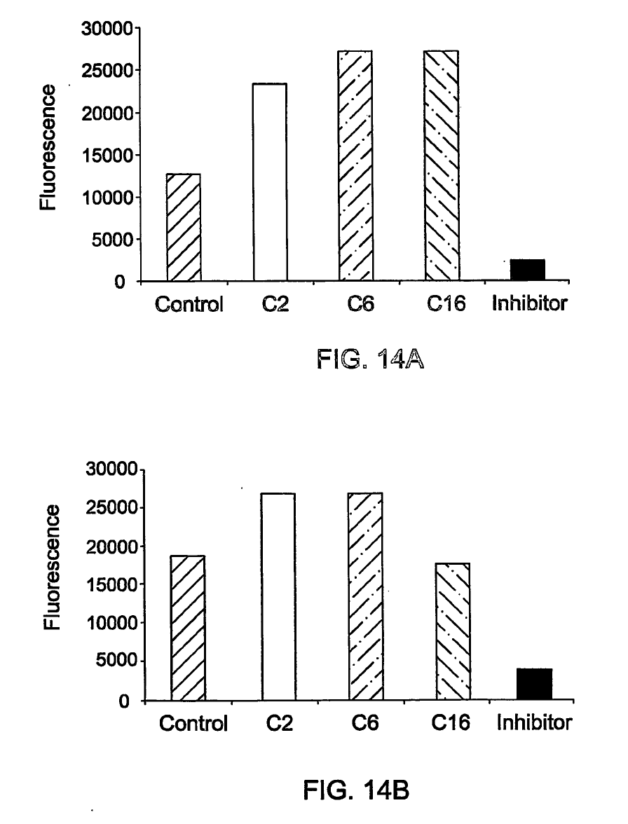

The activation of caspase-3 was measured in C-26 and OV-1063 cells treated with IC₅₀ values of different ceramides delivered as liposome formulations: EPC:^{2k}PEG-DSPE:C₂Cer; EPC: ^{2k}PEG -DSPE:C₆Cer; or EPC: ^{2k}PEG -DSPE:C₁₆Cer. OV-1063 cells that were treated for 5 hr with IC₅₀ values of liposomal ceramide formulations with the various ceramides (C₂Cer, C₆Cer, and C₁₆Cer) indicate the activation of caspase-3 (1.7, 1.9 and 1.8-fold increase for the three ceramides, respectively) (FIG. 13A). Also OV-1063 cells treated with “free” ceramides showed similar results to the liposomal ceramides (FIG. 13B). After 16 hr of treatment of OV-1063 cells with IC₅₀ values of liposome formulations with the three ceramides (C₂Cer, C₆Cer, and C₁₆Cer) there was, respectively, 1.8, 2.1 and 2.1-fold increase in caspase-3 activity as compared to the control, untreated cells (FIG. 14A). However, no activation of caspse-3 found in OV-1063-cells after 16 hr of treatment with free C₁₆ Cer (FIG. 14B)

To confirm that the results are indeed due to activation of caspase-3, the reversible Ac-DEVD-CHO inhibitor of caspase-3-like proteases was added to the control and treated samples. A drastic decrease in caspase-3 activity was found in ceramide-treated OV-1063 cells (but not of C-26 cells) after addition of Ac-DEVD-CHO inhibitor (FIGS. 14A and 14B, “inhibitor”). This data are in agreement with findings which have shown that colon cancer cells protect themselves from apoptosis by secreting soluble factor(s) [Liu, W. et al. Int. J. Cancer, 92:26-30 (2001)] and by aberrant activation of c-kit [Bellone, G. et al. Cancer Res., 21:2200-2206 (2001)]. These findings are also in agreement with the PS exposure and morphological changes described above.

Changes in Size Distribution of the Lipid Assemblies Studied by Dynamic Light Scattering: From in vitro to in vivo Administration of Ceramide Formulations:

Studies in cell cultures demonstrated that ceramides act as second messenger and biological modifiers. Indirect results suggest that increasing ceramide levels in tumors have beneficial and synergistic effect with anticancer chemotherapeutic drugs [Sechenkov et al., J. Natl. Cancer Inst., 93:347-357, (2001)]. However, so far the biological activity of the ceramides was not evaluated in vivo in spite their potential beneficial activity due to difficulties in their delivery. As mentioned above, ceramides by themselves are difficult to be dispersed in serum-free medium. It has been found that a mixture of ethanol and dodecane is useful to disperse ceramides homogeneously for their studies in cell culture [Hirabayashi et al, Supra, 1995]. When a volume of ceramides (C₂Cer, C₆Cer, and C₁₆Cer) in ethanol:dodecane (98:2 v/v) was diluted into 100 volumes of serum-free medium a milky translucent dispersion was formed.

Evaluation of size distribution of these different ceramide dispersions by dynamic light scattering (DLS) revealed that the diameter of the lipid particles made up of ethanol/dodecane/ceramide was 330 nm in the case of C₂Cer and C₆Cer ceramides, while particle size of C₁₆Cer in ethanol:dodecane dispersion was 790 nm (Table 11). However, when these ceramide dispersions were diluted further (final dilution 1:1000) in serum-containing medium, a 4-6 fold increase in particle size was observed. As a control, when the solvents ethanol:dodecane (98:2) were mixed by themselves with medium without ceramides, no particles were detected by DLS. Also, for comparison, when ceramide solutions in ethanol (without dodecane) were diluted 1:100 in serum-containing medium very large particles were observed by DLS (Table 11).

TABLE 11
Size distribution of various ceramide dispersions

| | | Ethanol:dodecane | |
|---|---|---|---|
| | Ethanol:dodecane (98:2) | (98:2) dispersion in | Ethanol dispersion |
| | dispersion in serum-free | serum-containing | in serum- |
| Type of the | medium (1:100) | medium (1:1000) | containing medium (1:100) |
| ceramide | Size (nm) | Size (nm) | Size (nm) |
| C₂ Cer | 330 | 1400 | 6514 |
| C₆ Cer | 330 | 2100 | 3780 |
| C₁₆ Cer | 790 | 2824 | 4740 |

An attempt to inject these ceramide dispersions in vivo was made. One mole/mouse of C₂Cer and C₆Cer dispersions in ethanol or in ethanol:dodecane (98:2) (required final blood concentration of about 3.3%) were injected to the Balb/c female mice (8 week old). It was found that the injection was very painful and inconvenient for the mice. Two mice that were injected with ethanol:dodecane (98:2) dispersions of C₆ Cer died during the injection. When the weight of mice was followed at three-day interval after injection, a decrease of about 5% from their initial weight was found.

In addition, 1 μmole/mouse of C₂Cer and C₆Cer dispersions in ethanol or in ethanol:dodecane (98:2) in isotonic BSA solution (BSA 2 mM, NaCl 112 mM, pH 7.4) was injected in order to reach the final ethanol or ethanol:dodecane blood concentration of about 3.3%. For the preparation of ceramide-BSA complexes the 33 μl of 30 mM stock of C₂Cer and C₆Cer dispersions were incubated for 30 min at 30° C. with 417 μl of 2 mM BSA solution in order to reach the ceramide/BSA mole ratio of 1/0.8, respectively [Hannun, Supra, 2000]. It was found that the injection was not tolerated by the mice, because, the injection was very painful and inconvenient for the mice and when the weight of mice was followed at three-day interval after injection, a decrease of about 5% from their initial weight was found.

Therefore, it was concluded that i.v. injection of ceramide alone in order to reach blood ceramide concentration of 2-4 mM (1-2 μmole/mouse) in ethanol (final blood concentration of about 3.3-6.7%), or in ethanol:dodecane (98:2) dispersion (final blood concentration of ethanol about 3.3-6.7% and of dodecane about 0.06-0.12%), or after adsorption to albumin (final blood concentration of ethanol about 2.4-4.8%) is not a suitable manner of administration to animals and probably neither to humans.

In vivo Antitumor Activity of Liposomes Comprising Lipopolymers and Biologically Active, Non-Liposome forming Lipids

It was previously shown that encapsulation of chemotherapeutic agents in liposomes which include lipopolymers, such as PEGylated lipids enhance their passive targeting to various tumors and inflammation sites as well as reducing their toxicity (due to liposome grafted ^{2K}PEG-DSPE effect on reducing liposome uptake by the reticuloendothelial system (RES) [Gabizon et al., Cancer Res., 54:987-992, (1994); Gabizon A, et al. Clin Pharmacokinet.42(5):419-36 (2003)]. This passive targeting of large unilamellar liposomes (≦100 nm) is due to their extravasation through impaired endothelium of the tumor blood vessels, which in many tumor tissues are enriched due to the angiogenesis in primary and metastatic tumors.

In vivo toxicity and antitumor efficacy of LUV comprised of EPC or HSPC, ^{2k}PEG-DSPE and C₆ Cer was evaluated. It was found that these lipid assemblies were non-toxic for mice at the doses injected.

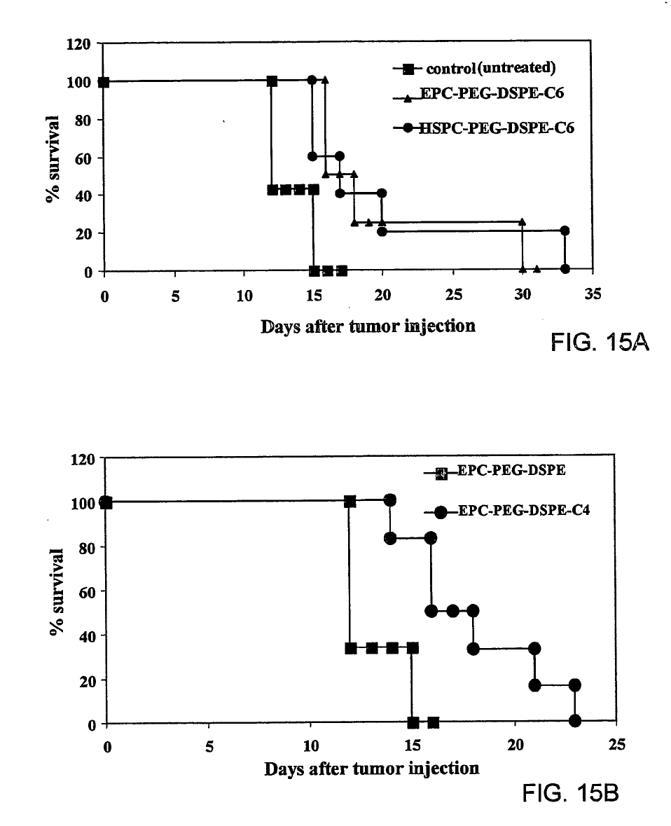

Large unilamellar vesicles comprised of EPC or HSPC, C₆ Cer and stabilized by ^{2k}PEG-DSPE were evaluated for anti-tumor efficacy on tumor-bearing (C₂₆ colon carcinoma) mice as compared to tumor-bearing untreated (control) mice group. The survival curves of mice inoculated with C-26 colon carcinoma cells i.p. and treated i.v. with liposomal C₆ Cer is presented in FIG. 15A. Treatment began at day 3 after inoculation of the mice with 10⁶ tumor cells. It was found that survival of treated animals with liposomal EPC/C₆ Cer was 19 days and when animals were treated with HSPC/C₆ Cer it was 18 days, which corresponds, respectively to 36.7% increase in life span (ILS) (p<0.001) and to 28.6% ILS (p<0.0045 (Table 12A, FIG. 15A).

TABLE 12A
Therapeutic efficacy of SSL-C₆ against C-26 tumor mice

| | | | Survival | |
|---|---|---|---|---|
| Treatment | Dose | No. of mice | (days) | ILS (%) |
| Control (not treated) | | 7 | 14 | |
| EPC-^{2K}PEG-DSPE-C₆ Cer | 2 μmole | 8 | 19 | 35.7 |
| (69.5:7.5:23) | (C₆ Cer) | | | |
| | 6 μmole | | | |
| | (PL) | | | |
| HSPC-^{2K}PEG-DSPE-C₆ Cer | 1 μmole | 5 | 18 | 28.6 |
| (81:7.5:11.5) | (C₆ Cer) | | | |
| | 6 μmole | | | |
| | (PL) | | | |

For comparison the tumor-suppressive activity of the EPC: C₄Cer LUV lacking ^{2K}PEG-DSPE was determined. We found that median survival of mice was 16 days and ILS was insignificant (p<0.064).

Also, tumor-suppressive activity of the EPC: ^{2K}PEG-DSPE:C₄ Cer LUV was determined. Treatment began at day 3 after i.p. inoculation of 10⁶ tumor cells at the dose of C₄ Cer of 2 μmole per mice and was repeated after one weak and again after additional 10 days at the dose of 1 μmole per mice. It was found that animals treated with control (ceramide lacking) liposomes (SSL) had a same median survival time of 14 days as the untreated (control) group (Table 12A). Animals treated with C₄ Cer containing liposomes showed a median survival time of 17 days which correspond to 20.7% increase in survival over control liposomes (p<0.0055, Table 12B and FIG. 15B). Thus it may be concluded that treatment of tumor-bearing subjects with PEGylated liposomes containing C₄ or C₆ ceramide has antitumor activity (Table 12B, FIG. 15B).

TABLE 12B
Therapeutic efficacy of SSL-C₄ against C-26 tumor in mice

| | | No. of | Survival | |
|---|---|---|---|---|
| Treatment | Concentration | mice | (days) | ILS (%) |
| Control SSL | 6 μmole (PL) | 6 | 14 | |
| EPC-^{2K}PEG-DSPE- | 2 μmole (C₄ | 6 | 17 | 129 |
| C₄ Cer (69.5:7.5:23) | Cer) 6 μmole | | | |
| | (PL) | | | |

Previously it was reported that by increasing endogenous ceramide levels it was possible to improve the efficacy of established anticancer agents [Cabot M. C. (1997) Supra]. The tumor suppressive activity of the liposomal ceramide formulations as stand-alone drugs was evaluated and surprisingly, even without optimization and without established additional anticancer drug, it was found that liposomal ceramides, such as liposomal C₄ Cer and C₆ Cer, prolonged the survival of the tumor-bearing mice and slow down tumor growth.

Pharmacokinetic Studies in Mice

In order to be efficacious the C₆Cer has to reach and get into the tumor cells. Pharmacokinetics and biodistribution of ¹⁴C₆ Cer and 3H DPPC labeled PC:C₆ Cer and PC:^{2K}PEG-DSPE:C₆ Cer LUV in normal and tumor-bearing mice were studied. In addition, the effect of type of liposome-forming lipid and of the steric stabilizer ^{2k}PEG-DSPE on the rate of release of C₆Cer, from the liposomes in the blood and level in various tissues including the tumor was determined.

For this, liposomes of various compositions were doubly labeled with ¹⁴C₆ as a marker for C₆Cer and with 3H DPPC as a marker for the liposome-forming PC (see Materials and Methods). Table 14 represents total radioactivity and molar doses that were injected through the tail vein of Balb/C female mice.

TABLE 13
Injected doses of radiolabelled lipid assemblies

| | Injected | | | |
|---|---|---|---|---|
| | dose of | | | |
| | C6Cer/PL | ¹⁴C₆Cer | ³H-DPPC | C₆Cer/PL |
| Lipid assembly | μmole/ | radioactivity | radioactivity | ratio |
| composition | μmole | Dpm/μmole | Dpm/μmole | Dpm/dpm |
| EPC/^{2k}PEG- | 0.74/4.5 | 10 × 10⁵ | 4.1 × 10⁵ | 2.43 |
| DSPE/C₆Cer | | | | |
| (81:7.5:11.5) | | | | |
| EPC/C₆Cer | 0.79/4.2 | 9.77 × 10⁵ | 3.57 × 10⁵ | 2.73 |
| (88.5:11.5) | | | | |
| HSPC/^{2k}PEG- | 0.81/6.7 | 10.37 × 10⁵ | 4.9 × 10⁵ | 2.12 |
| DSPE/C₆Cer | | | | |
| (81:7.5:11.5) | | | | |
| HSPC/C₆Cer | 0.80/5.0 | 10.5 × 10⁵ | 3.8 × 4 × 10⁵ | 2.76 |
| (88.5:11.5) | | | | |

The release of C₆Cer of the LUV in plasma in vivo was quantified according to the approach described by Amselem et al. [Amselem S., Cohen R, Barenholz Y, Chem. Phys. Lipids. 64:219-237 (1993)]. Specifically, following injection of the different doubly labeled ¹⁴C₆Cer and ³H DPPC liposomal formulations, blood samples were collected at predefined time points and plasma content was analyzed for ¹⁴C₆Cer and ³H DPPC.

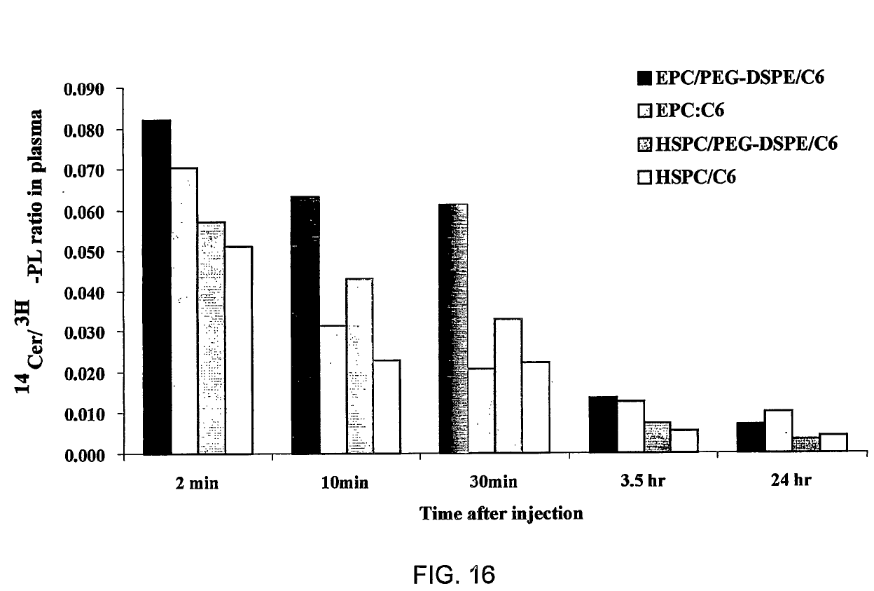

FIG. 16 shows that the clearance of C₆Cer is slowed down by ^{2k}PEG-DSPE. Specifically, 30 min after injection 10% of ¹⁴C₆Cer remained associated with liposomes composed of EPC:^{2k}PEG-DSPE:C₆Cer as compared to only 3.2% of ¹⁴C₆ Cer remaining in EPC:C₆ Cer LUV (lacking ^{2K}PEG-DSPE):C₆ Cer.

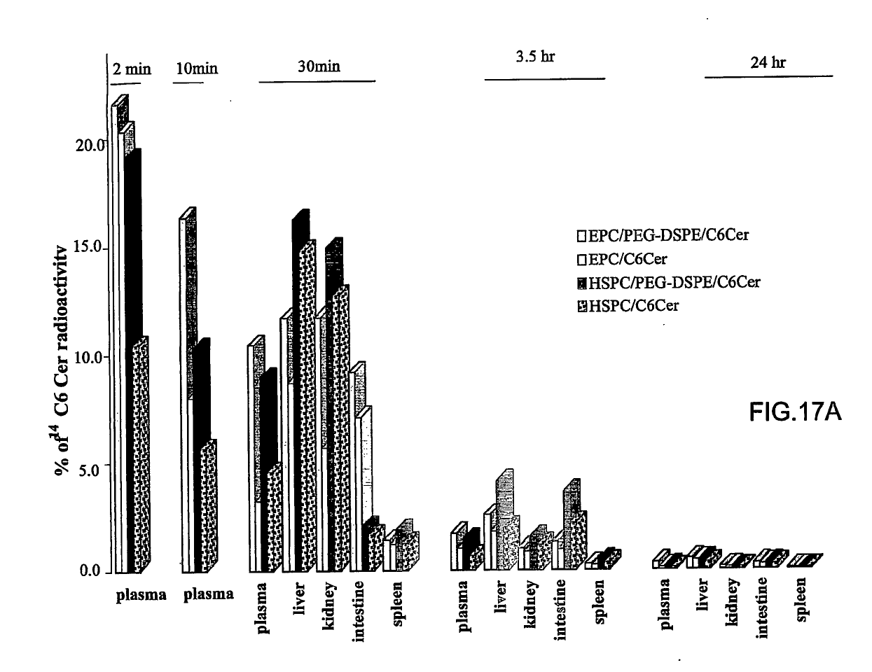

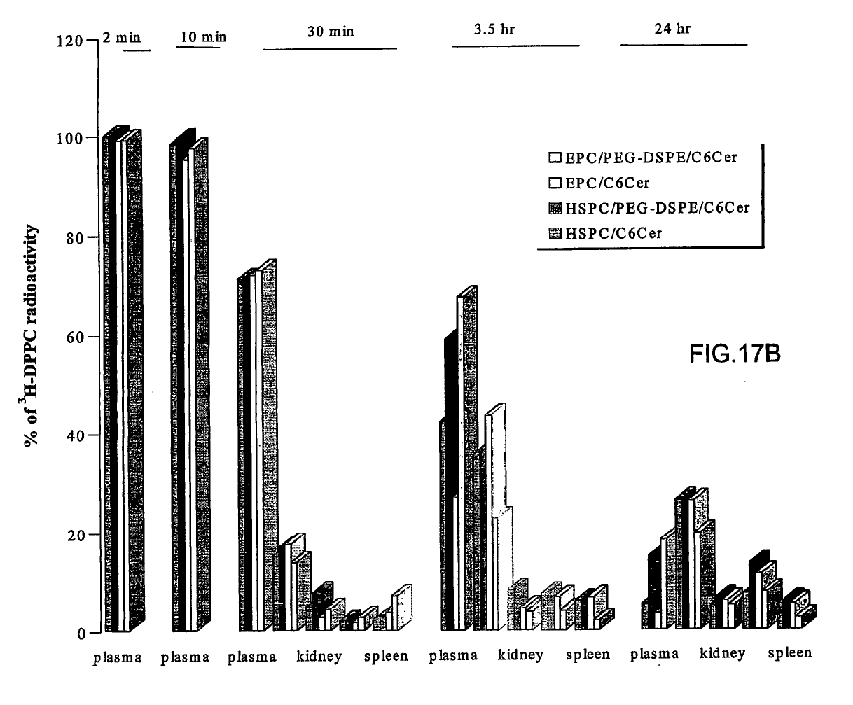

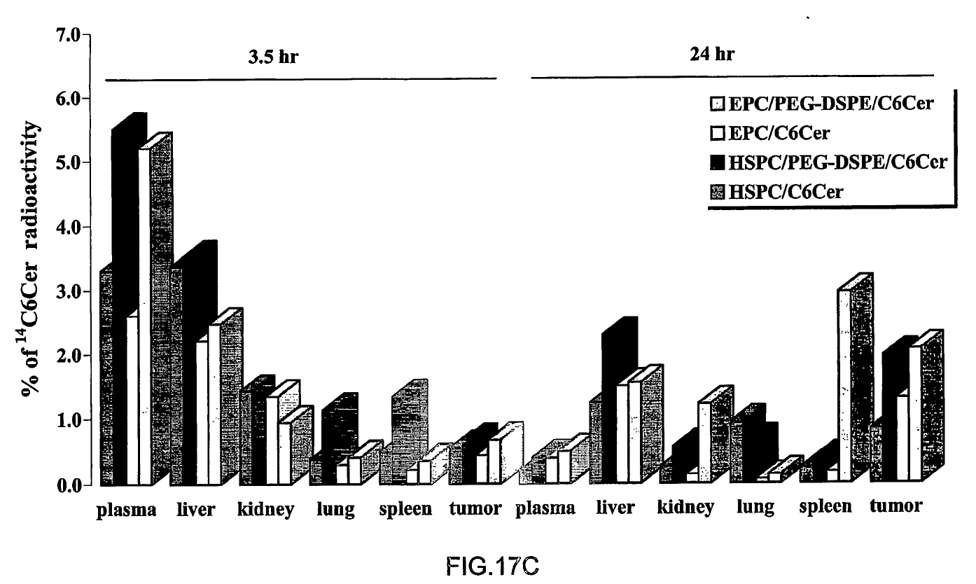

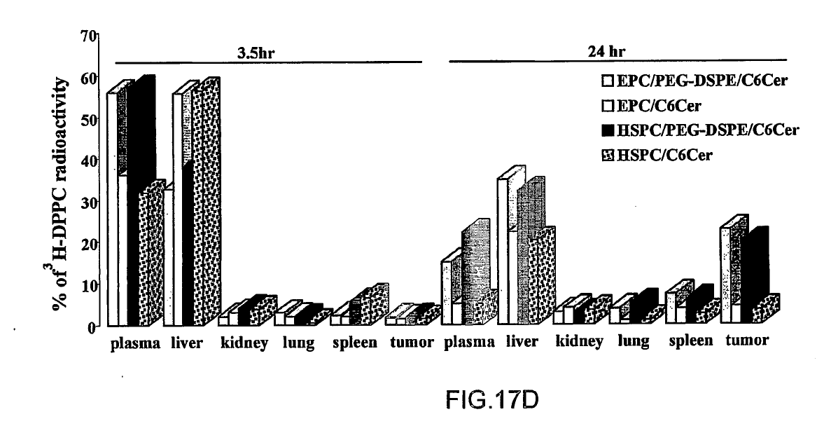

In addition, it was established that 30% of ¹⁴C₆Cer derived from liposomal EPC/^{2k}PEG-DSPE and 23% of ¹⁴C₆Cer derived from liposomal EPC were localized at different organs (FIG. 17A). The clearance of ¹⁴C₆Cer delivered via liposomes formed from HSPC alone or in combination with the steric stabilizer ^{2k}PEG-DSPE was also determined. Specifically, 30 min after injection 8.9% of ¹⁴C₆Cer from liposomes composed from HSPC:^{2k}PEG-DSPE:C₆Cer remained associated with the liposomes as compared to only 4.6% of ¹⁴C₆Cer from liposomes composed from HSPC:C₆Cer. In addition, 35% and 30% of ¹⁴C₆Cer derived from either HSPC/^{2k}PEG-DSPE or HSPC liposomes, respectively, were localized in different organs (FIG. 17A). In liver and spleen the clearance rate was slower during the 30 min after the injection and leakage increased significantly at the interval between 30 min to 3.5 hr post injection (FIG. 17A vs. FIG. 17B).

The above results confirmed results presented herein that ^{2K}PEG-DSPE when present in liposome slows down the clearance of ¹⁴C₆ Cer delivered via LUV. 6.8% and 11.1% of ¹⁴C₆ Cer derived from liposomes composed from EPC/^{2k}PEG-DSPE or HSPC/^{2k}PEG-DSPE respectively remained in plasma and organs 3.5 hr post injection as compared to only 4.4% and 6.3% of ¹⁴C₆ Cer derived from liposomes composed. from EPC or HSPC and lacking ^{2K}PEG-DSPE. Trace amounts of ¹⁴C₆ Cer from the various liposomes were found in plasma and organs 24 hr post injection (FIG. 17A).

The pharmacokinetics of ³H DPPC (the liposome-forming lipid marker) was also analyzed and found to resemble what is well-established for clearance of SSL. Three and a half hours after injection almost all (100%) liposomes recovered in blood and organs, however, the plasma content and bio-distribution depend on the liposome type. Specifically, 67% and 59% of ³H DPPC of PEGylated liposomes composed from EPC/^{2k}PEG-DSPE or HSPC/^{2k}PEG-DSPE remained in plasma 3.5 hr of post injection, as compared to only 27% and 32% of ³H DPPC from liposomes composed from C₆Cer and EPC or HSPC but lacking lipopolymer, respectively (FIG. 17B). The bio-distribution of ³H DPPC was also different. While with liposomes formed from EPC:C₆Cer or HSPC:C₆Cer 43% and 36%, respectively, of ³H DPPC were found in the liver, when ^{2K}PEG-DSPE was included in the liposomes 23% or 32% of the ³H DPPC, respectively, were found in the liver (FIG. 17B). Twenty four hours post-injection 18% and 15% of ³H DPPC from sterically stabilized liposomes based on EPC or HSPC liposome forming lipids as compared to only 3% and 5% derived from of liposomes composed from EPC or HSPC and lacking ^{2K}PEG-DSPE (FIG. 17B).

Biodistribution of various liposomes which were doubly labeled with ¹⁴C₆ Cer and with ³H DPPC was evaluated also in tumor-bearing mice. FIG. 17C shows that 24 hr post injection 2% of the ¹⁴C₆Cerf from total injected dose reached the tumor implanted subcutaneously into the left flank of the female Balb/c mice. Moreover, accumulation of ⁴C₆Cer in tumors was obtained between 3.5 and 24 ht post injection (FIG. 17C) compared with less than 0.5% present in plasma at the same time. The tumor levels of ⁴C₆Cer derived of SSL were higher in comparison to levels of C₆Cer derived of LUV lacking ^{2K}PEG-DSPE. (FIG. 17C). Continuous accumulation (between 3.5 hr and 24 hr) of ³H DPPC in tumors which was derived by SSL was observed during the experiment (FIG. 17D). On the other hand no such accumulation of ³H DPPC was observed when mice were injected with LUV lacking ^{2K}PEG-DSPE. (FIG. 17D). Percent of injected dose derived of SSL liposomes composed from EPC at 3.5 and 24 hr post injection accumulated in the tumor increased from 1.8 to 22.8 (12.6 fold increase) which was similar to liver accumulation and at a higher rate than in all other tested organs (FIG. 17D). On the other hand % of injected dose derived by EPC:C₆ Cer LUV lacking ^{2K}PEG-DSPE at 3.5 and 24 hr post injection increased to a much lower extent, from 1.6 to 4.3 (only 2.6 fold increase) which was at a lower rate than in liver and similar to other tested organs (FIG. 17D). These finding demonstrates that PC:^{2K}PEG-DSPE:C₆Cer LUV can extravasate and accumulate into tumor tissues. This explain the antitumor activity of these liposomes as described in FIGS. 15A and 15B.

1. A lipid assembly, being an organized collection of lipids, comprising:
(a) a biologically active lipid having a hydrophobic region and a polar headgroup, wherein the atomic mass ratio between the headgroup and hydrophobic region is less than 0.3;
(b) a lipopolymer having a hydrophobic lipid region and a hydrophilic polymer headgroup, wherein the atomic mass ratio between the headgroup and hydrophobic region is at least 1.5; the lipid assembly being chemically and physically stable under storage conditions of 4° C. in biological fluids, for at least six months.

2. The lipid assembly of claim 1, comprising a lipid matrix, the lipid matrix comprising a lipid or a combination of lipids having an additive packing parameter in the range of 0.74-1.0.

3. The lipid assembly of claim 1, having a level of water tightly bound to said lipopolymer headgroup of at least about 60 molecules of water per lipopolymer headgroup.

4. The lipid assembly of claim 2, wherein said biologically active lipid has a packing parameter which is greater than 1.

5. The lipid assembly of claim 2, wherein said biologically active lipid is selected from ceramides, ceramines, sphinganines, sphinganine-1-phosphate, di- or tri-alkylshpingosines and their structural analogs.

6. The lipid assembly of claim 5, wherein said biologically active lipid has the following general formula (I): 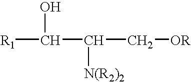
wherein
R₁ represent a C₂-C₂₆, saturated or unsaturated, branched or unbranched, aliphatic chain, the aliphatic chain may be substituted with one or more hydroxyl or cycloalkyl groups and may consist of a cycloalkylene moiety;
R₂ which may be the same or different, represents a hydrogen, a C₁-C₂₆ saturated or unsaturated, branched or unbranched chain selected from aliphatic, aliphatic carbonyl; a cycloalkylene-containing aliphatic chain, the aliphatic chain may be substituted with an aryl, arylalkyl or arylalkenyl group;
R₃ represents a hydrogen, a methyl, ethyl, ethenyl or a phosphate group.

7. The lipid assembly of claim 6, wherein said biologically active lipid is a C₂-C₂₆ ceramide.

8. The lipid assembly of claim 6, wherein said biologically active lipid is N,N-dimethylsphingosine (DMS).

9. (canceled)

10. The lipid assembly of claim 1, wherein said lipopolymer comprises a polymer headgroup selected from polyethylene glycol (PEG), polysialic acid, polylactic acid, polyglycolic acid, apolylactic-polyglycolic acid, polyvinyl alcohol, polyvinylpyrrolidone, polymethoxazoline, polyethyloxazoline, polyhydroxyethyloxazoline, polyhydroxypropyloxazoline, polyaspartamide, polyhydroxypropyl methacrylamide, polymethacrylamide, polydimethylacrylamide, polyvinylmethylether, polyhydroxyethyl acrylate, derivatized celluloses.

11. The lipid assembly of claim 9, wherein said polymer headgroup is polyethylene glycol (PEG) having an atomic mass in the range of about 750 Da to about 20,000 Da.

12. (canceled)

13. The lipid assembly of claim 10, wherein said PEG has an atomic mass of 2,000 Da (2 kPEG).

14. The lipid assembly of claim 2, wherein said lipid matrix comprises a phospholipid.

15. (canceled)

16. The lipid assembly of claim 12, wherein said phospholipid is a glycerophospholipid selected from phosphatidylglycerol (PG), phosphatidylcholine (PC), phosphatidic acid (PA), phosphatidylinositol (PI), phosphatidylserine (PS) and sphingomyelin (SPM) and derivatives of the same.

17. The lipid assembly of claim 2, wherein said lipid matrix comprises a cationic lipid.

18. The lipid assembly of claim 17, wherein said cationic lipid is a monocationic lipid having a headgroup selected from 1,2-dimyristoyl-3-trimethylammonium propane (DMTAP) 1,2-dioleyloxy-3-(trimethylamino) propane (DOTAP); N-[1-(2,3,-ditetradecyloxy)propyl]-N,N-dimethyl-N-hydroxyethylammonium bromide (DMRIE); N-[1-(2,3,-dioleyloxy)propyl]-N,N-dimethyl-N-hydroxy ethyl-ammonium bromide (DORIE); N-[1-(2,3-dioleyloxy) propyl]-N,N,N-trimethylammonium chloride (DOTMA); 3β[N-(N′,N′-dimethylaminoethane)carbamoly]cholesterol (DC-Chol); and dimethyl-dioctadecylammonium (DDAB).

19. The lipid assembly of claim 18, wherein said cationic lipid is a polycationic lipid having a headgroup selected from spermine or spermidine.

20. The lipid assembly of claim 19, wherein said polycationic lipid is N-[2-[[2,5-bis[3-aminopropyl)amino]-1-oxopentyl]amino]ethyl]-N,N-dimethyl-2,3-bis[(1-oxo-9-octadecenyl)oxy]-1-propanaminium (DOSPA) or ceramide carbamoyl spermine (CCS).

21-25. (canceled)

26. A pharmaceutical composition comprising a physiologically acceptable carrier and an amount of a stable lipid assembly, the amount being sufficient to achieve a biological effect at a target site, the lipid assembly comprising:
(a) a biologically active lipid having a hydrophobic region and a polar headgroup, wherein the atomic mass ratio between the headgroup and hydrophobic region is less than 0.3;
(b) a lipopolymer having a hydrophobic lipid region and a hydrophilic polymer headgroup, wherein the atomic mass ratio between the headgroup and hydrophobic region is at least 1.5; the lipid assembly being chemically and physically stable under storage conditions of 4° C. in biological fluids, for at least six months.

27-53. (canceled)

54. A method for the treatment or prevention of a disease, disorder or pathological condition comprising providing an individual in need of said treatment, in a manner so as to achieve a therapeutic effect, an effective amount of a stable lipid assembly comprising:
(a) a biologically active lipid having a hydrophobic region and a polar headgroup, wherein the atomic mass ratio between the headgroup and hydrophobic region is less than 0.3;
(b) a lipopolymer having a hydrophobic lipid region and a polymer headgroup, wherein the atomic mass ratio between the headgroup and hydrophobic region is at least 1.5.

55-80. (canceled)
